SEQUENCE LISTING (96 sequences)

SEQ ID NO: 1 - protein, 42 aa, Artificial Sequence
  Synthetically made according to the invention
TFIVGDQISFADYNLLDLLLIHEVLAPGCLDAFPLLSAYVGR

SEQ ID NO: 2 - protein, 12 aa, Artificial Sequence
  Synthetically made according to the invention
MPPYTVVYFPVR

SEQ ID NO: 3 - protein, 5 aa, Artificial Sequence
  Synthetically made according to the invention
DDYVK

SEQ ID NO: 4 - protein, 19 aa, Artificial Sequence
  Synthetically made according to the invention
DQQEAALVDMVNDGVEDLR

SEQ ID NO: 5 - protein, 16 aa, Artificial Sequence
  Synthetically made according to the invention
FQDGDLTLYQSNTILR

SEQ ID NO: 6 - protein, 10 aa, Artificial Sequence
  Synthetically made according to the invention
ASCLYGQLPK

SEQ ID NO: 7 - protein, 18 aa, Artificial Sequence
  Synthetically made according to the invention
AFLASPEYVNLPINGNGK

SEQ ID NO: 8 - protein, 11 aa, Artificial Sequence
  Synthetically made according to the invention
MLLADQGQSWK

SEQ ID NO: 9 - protein, 6 aa, Artificial Sequence
  Synthetically made according to the invention
LSARPK

SEQ ID NO: 10 - protein, 7 aa, Artificial Sequence
  Synthetically made according to the invention
TLGLYGK

SEQ ID NO: 11 - protein, 15 aa, Artificial Sequence
  Synthetically made according to the invention
EEVVTVETWQEGSLK

SEQ ID NO: 12 - protein, 20 aa, Artificial Sequence
  Synthetically made according to the invention
ALPGQLKPFETLLSQNQGGK

SEQ ID NO: 13 - protein, 13 aa, Artificial Sequence
  Synthetically made according to the invention
YISLIYTNYEAGK

SEQ ID NO: 14 - protein, 22 aa, Artificial Sequence
  Synthetically made according to the invention
HGEVCPAGWKPGSDTIKPDVQK

SEQ ID NO: 15 - protein, 16 aa, Artificial Sequence
  Synthetically made according to the invention
QGGLGPMNIPLVSDPK

SEQ ID NO: 16 - protein, 8 aa, Artificial Sequence
  Synthetically made according to the invention
ADEGISFR

SEQ ID NO: 17 - protein, 8 aa, Artificial Sequence
  Synthetically made according to the invention
DISLSDYK

SEQ ID NO: 18 - protein, 10 aa, Artificial Sequence
  Synthetically made according to the invention
LVQAFQFTDK

SEQ ID NO: 19 - protein, 9 aa, Artificial Sequence
  Synthetically made according to the invention
IGHPAPNFK

SEQ ID NO: 20 - protein, 24 aa, Artificial Sequence
  Synthetically made according to the invention
LNCQVIGASVDSHFCHLAWVNTPK

SEQ ID NO: 21 - protein, 25 aa, Artificial Sequence
  Synthetically made according to the invention
YVVFFFYPLDFTFVCPTEIIAFSDR

SEQ ID NO: 22 - protein, 7 aa, Artificial Sequence
  Synthetically made according to the invention
MSSGNAK

SEQ ID NO: 23 - protein, 10 aa, Artificial Sequence
  Synthetically made according to the invention
TIAQDYGVLK

SEQ ID NO: 24 - protein, 11 aa, Artificial Sequence
  Synthetically made according to the invention
ATAVMPDGQFK

SEQ ID NO: 25 - protein, 7 aa, Artificial Sequence
  Synthetically made according to the invention
GMPVTAR

SEQ ID NO: 26 - protein, 22 aa, Artificial Sequence
  Synthetically made according to the invention
MPGGLLLGDVAPNFEANTTVGR

SEQ ID NO: 27 - protein, 12 aa, Artificial Sequence
  Synthetically made according to the invention
DFTPVCTTELGR

SEQ ID NO: 28 - protein, 9 aa, Artificial Sequence
  Synthetically made according to the invention
VVFVFGPDK

SEQ ID NO: 29 - protein, 17 aa, Artificial Sequence
  Synthetically made according to the invention
LIALSIDSVEDHLAWSK

SEQ ID NO: 30 - protein, 14 aa, Artificial Sequence
  Synthetically made according to the invention
ELAILLGMLDPAEK

SEQ ID NO: 31 - protein, 11 aa, Artificial Sequence
  Synthetically made according to the invention
LSILYPATTGR

SEQ ID NO: 32 - protein, 8 aa, Artificial Sequence
  Synthetically made according to the invention
VATPVDWK

SEQ ID NO: 33 - protein, 7 aa, Artificial Sequence
  Synthetically made according to the invention
NFDEILR

SEQ ID NO: 34 - protein, 9 aa, Artificial Sequence
  Synthetically made according to the invention
LPFPIIDDR

SEQ ID NO: 35 - protein, 11 aa, Artificial Sequence
  Synthetically made according to the invention
VVISLQLTAEK

SEQ ID NO: 36 - protein, 13 aa, Artificial Sequence
  Synthetically made according to the invention
DINAYNCEEPTEK

SEQ ID NO: 37 - protein, 7 aa, Artificial Sequence
  Synthetically made according to the invention
LAPEFAK

SEQ ID NO: 38 - protein, 17 aa, Artificial Sequence
  Synthetically made according to the invention
DGDSVMVLPTIPEEEAK

SEQ ID NO: 39 - protein, 17 aa, Artificial Sequence
  Synthetically made according to the invention
FHDFLGDSWGILFSHPR

SEQ ID NO: 40 - protein, 12 aa, Artificial Sequence
  Synthetically made according to the invention
ALPESLGQHALR

SEQ ID NO: 41 - protein, 16 aa, Artificial Sequence
  Synthetically made according to the invention
DENATLDGGDVLFTGR

SEQ ID NO: 42 - protein, 16 aa, Artificial Sequence
  Synthetically made according to the invention
DYAVSTVPVADGLHLK

SEQ ID NO: 43 - protein, 10 aa, Artificial Sequence
  Synthetically made according to the invention
GAEILADTFK

SEQ ID NO: 44 - protein, 8 aa, Artificial Sequence
  Synthetically made according to the invention
GEEVDVAR

SEQ ID NO: 45 - protein, 12 aa, Artificial Sequence
  Synthetically made according to the invention
QHQLYVGVLGSK

SEQ ID NO: 46 - protein, 10 aa, Artificial Sequence
  Synthetically made according to the invention
TPEEYPESAK

SEQ ID NO: 47 - protein, 7 aa, Artificial Sequence
  Synthetically made according to the invention
HDELTYF

SEQ ID NO: 48 - protein, 12 aa, Artificial Sequence
  Synthetically made according to the invention
SQVVAGTNYFIK

SEQ ID NO: 49 - protein, 21 aa, Artificial Sequence
  Synthetically made according to the invention
VFQSLPHENKPLTLSNYQTNK

SEQ ID NO: 50 - protein, 12 aa, Artificial Sequence
  Synthetically made according to the invention
VHVGDEDFVHLR

SEQ ID NO: 51 - protein, 8 aa, Artificial Sequence
  Synthetically made according to the invention
MSQAEFEK

SEQ ID NO: 52 - protein, 6 aa, Artificial Sequence
  Synthetically made according to the invention
AAEEVR

SEQ ID NO: 53 - protein, 20 aa, Artificial Sequence
  Synthetically made according to the invention
QATVGDINTERPGMLDFTGK

SEQ ID NO: 54 - protein, 16 aa, Artificial Sequence
  Synthetically made according to the invention
TKPSDEEMLFIYGHYK

SEQ ID NO: 55 - protein, 8 aa, Artificial Sequence
  Synthetically made according to the invention
WDAWNELK

SEQ ID NO: 56 - protein, 25 aa, Artificial Sequence
  Synthetically made according to the invention
MWGDLWLLPPASANPGTGTEAEFEK

SEQ ID NO: 57 - protein, 9 aa, Artificial Sequence
  Synthetically made according to the invention
MPAFAEFEK

SEQ ID NO: 58 - protein, 15 aa, Artificial Sequence
  Synthetically made according to the invention
GFGFGQGAGALVHSE

SEQ ID NO: 59 - protein, 10 aa, Artificial Sequence
  Synthetically made according to the invention
GLESTTLADK

SEQ ID NO: 60 - protein, 15 aa, Artificial Sequence
  Synthetically made according to the invention
GYGYGQGAGTLSTDK

SEQ ID NO: 61 - protein, 11 aa, Artificial Sequence
  Synthetically made according to the invention
GGEAAEAEAEK

SEQ ID NO: 62 - protein, 21 aa, Artificial Sequence
  Synthetically made according to the invention
MDPETCPCPSGGSCTCADSCK

SEQ ID NO: 63 - protein, 12 aa, Artificial Sequence
  Synthetically made according to the invention
SCCSCCPAECEK

SEQ ID NO: 64 - protein, 20 aa, Artificial Sequence
  Synthetically made according to the invention
GNDISSGTVLSDYVGSGPPK

SEQ ID NO: 65 - protein, 8 aa, Artificial Sequence
  Synthetically made according to the invention
LYEQLSGK

SEQ ID NO: 66 - protein, 14 aa, Artificial Sequence
  Synthetically made according to the invention
LYTLVLTDPDAPSR

SEQ ID NO: 67 - protein, 15 aa, Artificial Sequence
  Synthetically made according to the invention
NRPTSISWDGLDSGK

SEQ ID NO: 68 - protein, 8 aa, Artificial Sequence
  Synthetically made according to the invention
VLTPTQVK

SEQ ID NO: 69 - protein, 13 aa, Artificial Sequence
  Synthetically made according to the invention
YVWLVYEQDRPLK

SEQ ID NO: 70 - protein, 87 aa, Homo sapiens
MSQAEFEKAAEEVRHLKTKPSDEEMLFIYGHYKQATVGDINTERPGMLDFTGKAKWDAWNELKGTSKEDAMKAYINKVEELKKKYGI

SEQ ID NO: 71 - protein, 193 aa, Homo sapiens
MPNWGGGKKCGVCQKTVYFAEEVQCEGNSFHKSCFLCMVCKKNLDSTTVAVHGEEIYCKSCYGKKYGPKGYGYGQGAGTLSTDKGESLGIKHEEAPGHRPTTNPNASKFAQKIGGSERCPRCSQAVYAAEKVIGAGKSWHKACFRCAKCGKGLESTTLADKDGEIYCKGCYAKNFGPKGFGFGQGAGALVHSE

SEQ ID NO: 72 - protein, 187 aa, Homo sapiens
MPVDLSKWSGPLSLQEVDEQPQHPLHVTYAGAAVDELGKVLTPTQVKNRPTSISWDGLDSGKLYTLVLTDPDAPSRKDPKYREWHHFLVVNMKGNDISSGTVLSDYVGSGPPKGTGLHRYVWLVYEQDRPLKCDEPILSNRSGDHRGKFKVASFRKKYELRAPVAGTCYQAEWDDYVPKLYEQLSGK

SEQ ID NO: 73 - protein, 285 aa, Homo sapiens
MAGLGHPAAFGRATHAVVRALPESLGQHALRSAKGEEVDVARAERQHQLYVGVLGSKLGLQVVELPADESLPDCVFVEDVAVVCEETALITRPGAPSRRKEVDMMKEALEKLQLNIVEMKDENATLDGGDVLFTGREFFVGLSKRTNQRGAEILADTFKDYAVSTVPVADGLHLKSFCSMAGPNLIAIGSSESAQKALKIMQQMSDHRYDKLTVPDDIAANCIYLNIPNKGHVLLHRTPEEYPESAKVYEKLKDHMLIPVSMSELEKVDGLLTCCSVLINKKVDS

SEQ ID NO: 74 - protein, 68 aa, Homo sapiens
MDPETCPCPSGGSCTCADSCKCEGCKCTSCKKSCCSCCPAECEKCAKDCVCKGGEAAEAEAEKCSCCQ

SEQ ID NO: 75 - protein, 98 aa, Homo sapiens
MMCGAPSATQPATAETQHIADQVRSQLEEKENKKFPVFKAVSFKSQVVAGTNYFIKVHVGDEDFVHLRVFQSLPHENKPLTLSNYQTNKAKHDELTYF

SEQ ID NO: 76 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 77 - protein, 916 aa, Homo sapiens
MSYTLDSLGNPSAYRRVTETRSSFSRVSGSPSSGFRSQSWSRGSPSTVSSSYKRSMLAPRLAYSSAMLSSAESSLDFSQSSSLLNGGSGPGGDYKLSRSNEKEQLQGLNDRFAGYIEKVHYLEQQNKEIEAEIQALRQKQASHAQLGDAYDQEIRELRATLEMVNHEKAQVQLDSDHLEEDIHRLKERFEEEARLRDDTEAAIRALRKDIEEASLVKVELDKKVQSLQDEVAFLRSNHEEEVADLLAQIQASHITVERKDYLKTDISTALKEIRSQLESHSDQNMHQAEEWFKCRYAKLTEAAEQNKEAIRSAKEEIAEYRRQLQSKSIELESVRGTKESLERQLSDIEERHNHDLSSYQDTIQQLENELRGTKWEMARHLREYQDLLNVKMALDIEIAAYRKLLEGEETRFSTFAGSITGPLYTHRPPITISSKIQKPKVEAPKLKVQHKFVEEIIEETKVEDEKSEMEEALTAITEELAVSMKEEKKEAAEEKEEEPEAEEEEVAAKKSPVKATAPEVKEEEGEKEEEEGQEEEEEEDEGAKSDQAEEGGSEKEGSSEKEEGEQEEGETEAEAEGEEAEAKEEKKVEEKSEEVATKEELVADAKVEKPEKAKSPVPKSPVEEKGKSPVPKSPVEEKGKSPVPKSPVEEKGKSPVPKSPVEEKGKSPVSKSPVEEKAKSPVPKSPVEEAKSKAEVGKGEQKEEEEKEVKEAPKEEKVEKKEEKPKDVPEKKKAESPVKEEAVAEVVTITKSVKVHLEKETKEEGKPLQQEKEKEKAGGEGGSEEEGSDKGAKGSRKEDIAVNGEVEGKEEVEQETKEKGSGREEEKGVVTNGLDLSPADEKKGGDKSEEKVVVTKTVEKITSEGGDGATKYITKSVTVTQKVEEHEETFEEKLVSTKKVEKVTSHAIVKEVTQSD

SEQ ID NO: 78 - protein, 128 aa, Homo sapiens
MQIFVKTLTGKTITLEVEPSDTIENVKAKIQDKEGIPPDQQRLIFAGKQLEDGRTLSDYNIQKESTLHLVLRLRGGIIEPSLRQLAQKYNCDKMICRKCYARLHPRAVNCRKKKCGHTNNLRPKKKVK

SEQ ID NO: 79 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 80 - protein, 146 aa, Homo sapiens
MAGPLRAPLLLLAILAVALAVSPAAGSSPGKPPRLVGGPMDASVEEEGVRRALDFAVGEYNKASNDMYHSRALQVVRARKQIVAGVNYFLDVELGRTTCTKTQPNLDNCPFHDQPHLKRKAFCSFQIYAVPWQGTMTLSKSTCQDA

SEQ ID NO: 81 - protein, 114 aa, Homo sapiens
MVIRVYIASSSGSTAIKKKQQDVLGFLEANKIGFEEKDIAANEENRKWMRENVPENSRPATGYPLPPQIFNESQYRGDYDAFFEARENNAVYAFLGLTAPPGSKEAEVQAKQQA

SEQ ID NO: 82 - protein, 249 aa, Homo sapiens
MAPSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ

SEQ ID NO: 83 - protein, 304 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 84 - protein, 61 aa, Homo sapiens
MDPNCSCAAGDSCTCAGSCKCKECKCTSCKKSCCSCCPVGCAKCAQGCICKGASDKCSCCA

SEQ ID NO: 85 - protein, 916 aa, Homo sapiens; identical to SEQ ID NO: 77

SEQ ID NO: 86 - protein, 142 aa, Homo sapiens
MATKIDKEACRAAYNLVRDDGSAVIWVTFKYDGSTIVPGEQGAEYQHFIQQCTDDVRLFAFVRFTTGDAMSKRSKFALITWIGENVSGLQRAKTGTDKTLVKEVVQNFAKEFVISDRKELEEDFIKSELKKAGGANYDAQTE

SEQ ID NO: 87 - protein, 161 aa, Homo sapiens
MNLAISIALLLTVLQVSRGQKVTSLTACLVDQSLRLDCRHENTSSSPIQYEFSLTRETKKHVLFGTVGVPEHTYRSRTNFTSKYNMKVLYLSAFTSKDEGTYTCALHHSGHSPPISSQNVTVLRDKLVKCEGISLLAQNTSWLLLLLLSLSLLQATDFMSL

SEQ ID NO: 88 - protein, 140 aa, Homo sapiens
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQY

SEQ ID NO: 89 - protein, 44 aa, Homo sapiens
MSDKPDMAEIEKFDKSKLKKTETQEKNPLPSKETIEQEKQAGES

SEQ ID NO: 90 - protein, 61 aa, Homo sapiens
MDPNCSCATGGSCTCAGSCKCKECKCTSCKKSCCSCCPVGCAKCAQGCVCKGASEKCSCCA

SEQ ID NO: 91 - protein, 132 aa, Homo sapiens
MVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKA

SEQ ID NO: 92 - protein, 432 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 93 - protein, 260 aa, Homo sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 94 - protein, 1064 aa, Homo sapiens
KILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 95 - protein, 110 aa, Homo sapiens
MRFMTLLFLTALAGALVCAYDPEAASAPGSGNPCHEASAAQKENAGEDPGLARQAPKPRKQRSSLLEKGLDGAKKAVGGLGKLGKDAVEDLESVGKGAVHDVKDVLDSVL

SEQ ID NO: 96 - protein, 94 aa, Homo sapiens
MRTLAILAAILLVALQAQAEPLQARADEVAAAPEQIAADIPEVVVSLAWDESLAPKHPGSRKNMACYCRIPACIAGERRYGTCIYQGRLWAFCC

(19) United States
(12) Patent Application Publication
(10) Pub. No.: US 2013/0252834 A1
(43) Pub. Date: Sep. 26, 2013

(54) Diagnostic Methods

(71) Applicants: Loic Gerard Dayon, London (GB); Jean-Charles Sanchez, Geneva (CH); Joan Montaner Villalonga, ES (ES)

(72) Inventors: Loic Gerard Dayon, London (GB); Jean-Charles Sanchez, Geneva (CH); Joan Montaner Villalonga, ES (ES)

(73) Assignees: UNIVERSITE DE GENEVE, Geneva 4 (CH); ELECTROPHORETICS LIMITED, Cobham Surrey (GB)

(21) Appl. No.: 13/699,444

(22) PCT Filed: May 23, 2011
(86) PCT No.: PCT/GB11/00784

§ 371 Date: Nov. 21, 2012

(30) Foreign Application Priority Data
May 21, 2010 (GB) 1008541.3

(51) Int. Cl. G01N 33/68; C12N 9/00; C12N 9/10; C12Q 1/26; C12Q 1/48

(52) U.S. Cl. CPC G01N 33/6896; C12Q 1/26; C12Q 1/48; C12N 9/1088; C12N 9/0065
USPC 506/9; 435/25; 435/15; 506/12; 435/7.4; 506/18; 530/300

(57) ABSTRACT

The invention relates to a method of aiding the diagnosis of acute brain damage in a subject, said method comprising (i) assaying the concentration of at least one oxidative stress polypeptide selected from the group consisting of: PRDX1, PRDX6 and GSTP1 in a sample from said subject; and (ii) assaying the concentration of at least one further polypeptide selected from Panel A; (Hi) comparing the concentrations of (i) and (ii) to the concentrations of the polypeptides in a reference standard and determining quantitative ratios for said polypeptides; (iv) wherein a finding of a quantitative ratio of each of the assayed polypeptides in the sample to the polypeptides in the reference standard of greater than 1.3 indicates an increased likelihood of acute brain damage having occurred in said subject.

### FIELD OF THE INVENTION

The invention relates to aiding the diagnosis of acute brain injury. In particular the invention relates to aiding the diagnosis of stroke. Novel biomarkers and panels of biomarkers are described in the methods of the invention.

### BACKGROUND TO THE INVENTION

Stroke is a leading cause of death and disability in industrialized countries. The rapid diagnosis of an acute stroke is essential to triage suspected patients and transfer confirmed ones in specialized stroke units.

It is well known that non-cerebrovascular conditions can present with a clinical picture mimicking stroke, so that early accurate differentiation of such “mimics” from true stroke is essential to direct patients towards appropriate care. At present, the absence of a simple and widely available diagnostic test for acute cerebral ischemia remains a problem in the diagnosis (mostly based on clinical grounds and neuroimaging techniques) and management of stroke. In addition, the prognosis of stroke patients is relevant to rationalize the treatment and the follow-up.

The need for markers to diagnose a stroke and/or to predict probable course and outcome of the disease is therefore a major problem for the medical workforce (1).

Human cerebral microdialysis is an in vivo sampling technique to monitor the changes in composition of extracellular fluid (ECF) in the brain. Basically, a flexible microprobe is inserted into the patient's brain and a solution with composition very close to that of cerebrospinal fluid (CSF) is perfused (2). The probe simulates the function of a fenestrated capillary. The endogenous substances, that can pass the semi-permeable membrane situated at the probe tip, diffuse from the interstitial fluid to the microdialysis solution.

In the past few years, several studies measuring small molecules in human brain microdialysates, such as substrates (e.g., glucose), metabolites (e.g., pyruvate, lactate), and neurotransmitters (e.g., glutamate) were carried out (3-5). Conversely, few proteomic studies of these rare materials have been reported to date (6, 7). The ability to recover proteins depends on several physico-chemical factors such as their molecular weight, hydrophobicity/hydrophilicity, charge, shape, radius of gyration and interactions with other molecules. The structure of the microdialysis catheters, the pore size of the membrane, the flow rate, the temperature, and the diffusion properties of the proteins inside the perfused fluid influence both the protein and fluid recovery (8). As an example, in vitro recovery of protein S100-B (S100B), a 12 kDa calcium binding protein with important intracellular and extracellular function (9), was improved with catheter MW cut-off of 100 kDa with respect to formal cut-off value of 20 kDa (10). The accumulation of biological debris within the catheter was also shown to decrease recovery over time (8). Thus microdialysis approaches remain technically extremely challenging. Furthermore, they represent an invasive procedure requiring incision and access to the inner parts of the brain, which is an extremely specialised and difficult procedure to carry out.

In this context, Maurer et al. carried out a proteomic analysis of human brain microdialysate with two-dimensional gel electrophoresis and mass spectrometry (MS), and identified 27 proteins from the non-infarcted (i.e. contralateral (CT)) hemisphere of stroke patients (11). Many of those proteins were previously detected in CSF but few appeared to be exclusively present in the brain microdialysate. None appeared to show sufficient utility as a biomarker of stroke. In more recent research, microdialysate samples of patients with subarachnoid hemorrhage (SAH), developing or not a vasospasm, were compared (12). Glyceraldehyde-3-phosphate and heat-shock cognate 71 kDa proteins were respectively increased and decreased in the group that suffered a posterior vasospasm that may produce a cerebral infarction as a side effect. The authors concluded that these proteins might be used as early markers for the development of symptomatic vasospasm after SAH.

In view of the above, the identification of markers indicative of acute brain injury and/or the diagnosis of stroke remains an unanswered problem in the field.

The present invention seeks to overcome problem(s) associated with the prior art.

### DESCRIPTION OF THE INVENTION

Due to their permitting real-time monitoring and sampling in close proximity to the damaged tissue, human brain microdialysates are a highly valuable source material for the discovery of brain-specific biomarkers. Proteomic analysis of human brain microdialysis samples has been applied by the inventors to find innovative molecules for the diagnosis and prognosis of cerebrovascular disorders such as stroke.

These studies allowed the identification of particular biomarkers and further allowed them to be interrogated for association with stroke. The biomarkers could be further characterised in terms of their association with particular forms or elements of the injury such as the proximity to the core of the damaged region or other such property.

The insights gained from these demanding studies have permitted the identification of certain biomarkers for acute brain injury such as stroke. Thus the inventors have been able to devise methods for aiding diagnosis of such conditions as detailed herein.

Thus in one aspect the invention provides a method of aiding the diagnosis of acute brain damage in a subject, said method comprising

(i) assaying the concentration of at least one oxidative stress polypeptide selected from the group consisting of: PRDX1, PRDX6 and GSTP1 in a sample from said subject; and
(ii) assaying the concentration of at least one further polypeptide selected from Panel A;
(iii) comparing the concentrations of (i) and (ii) to the concentrations of the polypeptides in a reference standard and determining quantitative ratios for said polypeptides;
(iv) wherein a finding of a quantitative ratio of each of the assayed polypeptides in the sample to the polypeptides in the reference standard of greater than 1.3 indicates an increased likelihood of acute brain damage having occurred in said subject.

The oxidative stress polypeptide may be referred to as an oxidative stress related polypeptide.

Optionally the at least one oxidative stress polypeptide of (i) may be assayed in combination with the oxidative stress protein S100B.

The polypeptide is suitably an oxidative stress polypeptide. The polypeptide is suitably selected from the group consisting of PRDX1, PRDX6 and GSTP1. This group shares the common property of being oxidative stress proteins. These proteins are antioxidative enzymes. They are each connected by their involvement in the elimination of reactive oxygen species. Thus these polypeptides are conceptually related. Moreover, they are functionally related. These polypeptides are taught as a group for the first time as diagnostic of stroke. Thus one contribution made to the art by the current invention is to place this biologically connected group of polypeptides together into a single group being diagnostic indicators of stroke.

Optionally the group of PRDX1, PRDX6 and GSTP1 may include other protein(s) induced by oxidative stress. For example the group may include the protein S100B which is induced in oxidative stress. These polypeptides are taught as a group for the first time as diagnostic of stroke.

In addition to the common properties noted above, and in addition to the specific common utility taught here for the first time for this group, and in addition to the small and defined size of this cluster of polypeptides, it is important to note that they are also connected by virtue of being evidenced as direct interactors with one another. For example, these proteins have been demonstrated to be part of a single biological complex.

For example, PRDX1 and GSTP1 are implicated in similar redox protective mechanisms. Furthermore, they have been evidenced to interact together (Krapfenbauer 2003 Brain Res. 967 p 152). In addition, GSTP1 has been shown to reactivate oxidized PRDX6 (Schreibelt 2008 Free Radic. Biol. Med. 45 p 256). In addition, the formation of a complex has been biochemically demonstrated (Kim 2006 Cancer Res. 66 p 7136).

In addition to these powerful indications of common biological function, GSTP1 has been shown to reactivate oxidised PRDX6 (Manevich 2004 PNAS 101 p 3780). Moreover, complex formation between these polypeptides has also been proved (Ralat 2006 Biochemistry (Mosc.) 45 p 360).

Thus for at least these reasons the group consisting of: PRDX1, PRDX6 and GSTP1 forms a single invention, each member of this very small group being linked so as to form a single inventive concept. This concept may be characterised as the assay of oxidative stress proteins as an indicator of stroke. Alternatively this concept may be characterised as the teaching that assaying for a single biological assembly (i.e. the above described peroxiredoxin complex) can aid in the diagnosis of stroke. In order to define the invention in the most definite terms, the individual different molecular members of the complex are individually recited. However, it should be noted that these individual polypeptides share a technical relationship for the reasons given above. Thus each of the individual proteins mentioned share the special technical features of being in the same biological complex, contributing the same biological function, being in the same in vivo macromolecular assembly and other common properties as described. Thus the application relates to a single invention characterised by the new teaching connecting the members of this complex to the diagnosis of stroke.

Suitably step (i) comprises assaying the concentration of at least two oxidative stress polypeptide selected from the group consisting of: PRDX1, PRDX6 and GSTP1.

Suitably step (i) comprises assaying the concentration of each of the oxidative stress polypeptides PRDX1, PRDX6 and GSTP1

Suitably step (ii) may comprise measurement(s) of one or more of the panel of oxidative stress-related proteins described above as part of a larger panel in combination with proteins with other functions. For example this includes other proteins discovered in brain microdialysates.

Suitably step (ii) comprises assaying the concentration of at least one further polypeptide selected from Panel B.

Suitably step (ii) comprises assaying the concentration of at least one further polypeptide selected from Panel C.

Suitably step (ii) comprises assaying the concentration of at least one further polypeptide selected from enlarged panel ABC

Suitably step (ii) comprises assaying the concentration of at least one further polypeptide selected from Panel 1.

Suitably step (ii) comprises assaying the concentration of at least one further polypeptide selected from Panel 1H.

Suitably step (ii) comprises assaying the concentration of at least one further polypeptide selected from Panel 1C.

Suitably step (ii) comprises assaying the concentration of at least one further polypeptide selected from Panel 1A.

Suitably step (ii) comprises assaying the concentration of at least one further polypeptide selected from Panel 1B.

Suitably step (ii) comprises assaying the concentration of at least one further polypeptide selected from Panel 2.

Suitably step (ii) comprises assaying the concentration of at least one further polypeptide selected from Panel 2A.

Suitably step (ii) comprises assaying the concentration of at least one further polypeptide selected from Panel 2B.

Suitably step (ii) comprises assaying the concentration of at least two further polypeptides selected from said Panel.

Suitably step (ii) comprises assaying the concentration of at least four further polypeptides selected from said Panel.

Suitably assaying the concentration of at least one further marker from said panel is carried out.

Suitably the acute brain injury is stroke.

Suitably the sample is brain microdialysate fluid, cerebrospinal fluid, or blood.

Most suitably the sample is blood.

Suitably step (i) comprises assaying the concentration of PRDX1 in a sample from said subject.

Suitably the protein is detected by western blotting.

Suitably the protein is detected by bead suspension array or by planar array.

Suitably the protein is detected by isobaric protein tagging or by isotopic protein tagging.

Suitably the protein is detected by mass spectrometer-based assay.

In another aspect, the invention relates to use for diagnostic or prognostic applications relating to acute brain damage of a material which recognises, binds to or has affinity for a first and a second polypeptide or a fragment, variant or mutant thereof, wherein the first polypeptide is selected from PRDX1, PRDX6 and GSTP1 and the second polypeptide is selected from Panel A.

In another aspect, the invention relates to use for diagnostic or prognostic applications relating to stroke of a material which recognises, binds to or has affinity for a polypeptide or a fragment, variant or mutant thereof, wherein the polypeptide is selected from Panel 2.

In another aspect, the invention relates to use as described above of a combination of materials, each of which respectively recognises, binds to or has affinity for one or more of said polypeptide(s), or a fragment, variant or mutant thereof.

In another aspect, the invention relates to use as described above, in which the or each material is an antibody or antibody chip.

In another aspect, the invention relates to use as described above, in which the material is an antibody with specificity for one or more of said polypeptide(s), or a fragment, variant or mutant thereof.

In another aspect, the invention relates to an assay device for use in the diagnosis of acute brain damage, which comprises a solid substrate having a location containing a material, which recognizes, binds to or has affinity for a first and a second polypeptide or a fragment, variant or mutant thereof, wherein the first polypeptide is selected from PRDX1, PRDX6 and GSTP1 and the second polypeptide is selected from Panel A.

In another aspect, the invention relates to an assay device for use in the diagnosis of stroke, which comprises a solid substrate having a location containing a material, which recognizes, binds to or has affinity for a polypeptide, or a fragment, variant or mutant thereof, wherein the polypeptide is selected from Panel 2.

In an assay device as described above, suitably the material is an antibody or antibody chip.

Suitably the assay device has a unique addressable location for each antibody, thereby to permit an assay readout for each individual polypeptide or for any combination of polypeptides.

In another aspect, the invention relates to a kit for use in the diagnosis of stroke, comprising an assay device as described above, and means for detecting the amount of one or more of the polypeptides in a sample of body fluid taken from a subject.

More suitably the polypeptide is a peroxiredoxin. More suitably the polypeptide is PRDX1.

In another aspect, the invention relates to a method of diagnosis or prognostic monitoring of acute brain damage in a subject, said method comprising
(a) obtaining and extracting the proteins from a relevant tissue sample from an individual;
(b) digesting said proteins to produce a population of peptides;
(c) determining the abundance of one or more of said peptides listed in Table 14 using Selected Reaction Monitoring of one or more of the transitions listed in Table 15;
(d) comparing the abundance of said one or more peptides with a pre-determined peptide abundance associated with a diagnosis of acute brain damage; and
(e) determining whether the subject has suffered acute brain damage and/or that the acute brain damage is worsening or improving based on the differences in abundance of said one or more peptides.

Suitably the pre-determined peptide abundance is determined using a known amount of corresponding synthetic peptide selected from Table 14.

In another aspect, the invention relates to a preparation for making a diagnosis of acute brain damage or prognostic monitoring of a subject with acute brain damage comprising one or more synthetic peptides selected from the group listed in Table 14.

Suitably said one or more synthetic peptides are selected from:

| | |
|---|---|
| GSTP1 | TFIVGDQISFADYNLLDLLLIHEVLAPGCLDAFPLLSAYVGR |
| | MPPYTVVYFPVR |
| | DDYVK |
| | DQQEAALVDMVNDGVEDLR |
| | FQDGDLTLYQSNTILR |
| | ASCLYGQLPK |
| | AFLASPEYVNLPINGNGK |
| | MLLADQGQSWK |
| | LSARPK |
| | TLGLYGK |
| | EEVVTVETWQEGSLK |
| | ALPGQLKPFETLLSQNQGGK |
| | YISLIYTNYEAGK |
| PRDX1 | HGEVCPAGWKPGSDTIKPDVQK |
| | QGGLGPMNIPLVSDPK |
| | ADEGISFR |
| | DISLSDYK |
| | LVQAFQFTDK |
| | IGHPAPNFK |
| | LNCQVIGASVDSHFCHLAWVNTPK |
| | YVVFFFYPLDFTFVCPTEIIAFSDR |
| | MSSGNAK |
| | TIAQDYGVLK |
| | ATAVMPDGQFK |
| PRDX6 | GMPVTAR |
| | MPGGLLLGDVAPNFEANTTVGR |
| | DFTPVCTTELGR |
| | VVFVFGPDK |
| | LIALSIDSVEDHLAWSK |
| | ELAILLGMLDPAEK |
| | LSILYPATTGR |
| | VATPVDWK |
| | NFDEILR |
| | LPFPIIDDR |
| | VVISLQLTAEK |
| | DINAYNCEEPTEK |
| | LAPEFAK |
| | DGDSVMVLPTIPEEEAK |
| | FHDFLGDSWGILFSHPR |
| DDAH1 | ALPESLGQHALR |
| | DENATLDGGDVLFTGR |
| | DYAVSTVPVADGLHLK |
| | GAEILADTFK |
| | GEEVDVAR |
| | QHQLYVGVLGSK |
| | TPEEYPESAK |
| CYTB | HDELTYF |
| | SQVVAGTNYFIK |
| | VFQSLPHENKPLTLSNYQTNK |
| | VHVGDEDFVHLR |
| ACBP | MSQAEFEK |
| | AAEEVR |
| | QATVGDINTERPGMLDFTGK |
| | TKPSDEEMLFIYGHYK |
| | WDAWNELK |
| | MWGDLWLLPPASANPGTGTEAEFEK |
| | MPAFAEFEK |
| CSRP1 | GFGFGQGAGALVHSE |
| | GLESTTLADK |
| | GYGYGQGAGTLSTDK |
| MT3 | GGEAAEAEAEK |
| | MDPETCPCPSGGSCTCADSCK |
| | SCCSCCPAECEK |
| PEPB1 | GNDISSGTVLSDYVGSGPPK |
| | LYEQLSGK |
| | LYTLVLTDPDAPSR |
| | NRPTSISWDGLDSGK |
| | VLTPTQVK |
| | YVWLVYEQDRPLK |

In another aspect, the invention relates to a preparation as described above wherein each peptide contains one or more stable heavy isotopes selected from hydrogen, carbon, oxygen, nitrogen or sulphur.

In another aspect, the invention relates to a preparation as described above wherein said synthetic peptides are labelled with an isotopic or isobaric tag.

In another aspect, the invention relates to a preparation as described above for the diagnosis or prognostic monitoring of acute brain damage.

In another aspect, the invention relates to a preparation as described above wherein the acute brain damage is ischaemic stroke or transient ischaemic attack.

In another aspect, the invention relates to a method for aiding the diagnosis of stroke in a subject, said method comprising

(i) assaying the concentration of at least one oxidative stress polypeptide selected from the group consisting of: PRDX1, PRDX6 and GSTP1 in a sample from said subject;
(ii) comparing the concentration of (i) to the concentration of the polypeptide in a reference standard and determining a quantitative ratio for said polypeptide;
(iii) wherein a finding of a quantitative ratio of the polypeptide in the sample to the polypeptide in the reference standard of greater than 1.3 indicates an increased likelihood of stroke having occurred in said subject.

Certain method steps discussed herein require the assay of one or more ‘further polypeptide(s)’ in addition to other requirements of the methods. A further polypeptide is one which is different to the one or more polypeptide(s) already required to be assayed. This is important because some of the groups of polypeptides presented herein contain members which are common to other groups presented herein. Clearly the mention of a ‘further polypeptide’ is intended to impose the assay of an additional polypeptide in addition to any which are or have been already assayed according to an earlier part of the method. Thus if a method requires one of A/B/C to be assayed and requires the assay of a further polypeptide selected from A/B/D/E/F/G, then merely assaying A twice or B twice does not constitute assaying a ‘further’ polypeptide as set out herein; assaying A then B would constitute the assay of a further polypeptide; assaying A then D would constitute the assay of a further polypeptide and so on. Thus suitably the further polypeptide is an additional polypeptide; suitably the further polypeptide is different from each other polypeptide assayed in the same method.

Acute Brain Damage embraces any rapid onset insults or injuries to the brain. Acute brain damage may include traumatic brain injury. Acute brain damage may include the effects resulting from stroke such as ischemic stroke. Acute brain damage may include any other acute brain injury. In a preferred embodiment the acute brain injury is stroke; most preferably ischemic stroke.

The sample may be any suitable biological sample from a subject to be tested. The sample may be microdialysate fluid gathered from microdialyis of the brain. This has the advantage of being most closely associated with the site of possible injury.

The sample may be cerebrospinal fluid. This has the advantage of being more easily collected than microdialysate. This is therefore less demanding on the patient and on the skilled operator performing the collection.

The sample may be blood. This has the advantage of being easily collected in a minimally invasive manner. The collection of blood requires only ordinary commonly available equipment and modest training of the medical staff performing the collection.

The sample may be cleared blood (i.e. plasma or cleared plasma), where the red and white blood cells have been removed for example by centrifugation. These offer advantages of stabilising the sample and making it easier to store or handle, or even easier to analyse/assay.

Suitably the method(s) described do not involve the actual step of collection of the sample from the subject. Suitably the step of sample collection is omitted from the methods of the invention. Suitably the sample is previously collected. Suitably the methods are in vitro methods. Suitably the methods do not require the physical presence of the subject from whom the sample has been previously collected. Suitably the sample is an in vitro sample.

Plasma can be obtained relatively easily and may reflect the sub-proteomes of other organs, including the brain. Both candidate protein panels and gel based proteomics have previously been used in plasma and serum to identify possible biomarkers with some success.

One of the problems with the proteomic analysis of blood plasma with mass spectrometry, is the huge dynamic range of plasma proteins. Protein levels span an extraordinary 10 orders of magnitude, which makes the investigation of low(er) abundant proteins nearly impossible (Anderson and Anderson, 2002, Jacobs et al., 2005). The instrumental settings in the LC/MS/MS, where the most prominent peaks in a short period of time are chosen for fragmentation, do not allow for the identification and quantitation of low abundant proteins in unfractionated plasma due to the high abundance of serum albumin and other proteins. This is reflected in a low number of proteins identified. One approach to reduce the dynamic range is to deplete samples of the highest abundant proteins and in this case we exemplify this approach using an immunoaffinity column to remove albumin, transferrin, IgG, IgA, antitrypsin, and haptoglobin. The number of identifiable and quantifiable proteins could be increased considerably and relative protein levels were compared between different samples.

For certain assay formats, the sample according to the invention may be a processed plasma. This is advantageous when the sample is to be analysed by mass spectrometry. For example, plasma may be processed to remove highly abundant proteins, and thereby to increase the number of detectable proteins, or to increase the detectability of proteins present in low absolute concentrations. Techniques for depletion of highly abundant proteins from plasma are well-known in the art. In particular, a multiple affinity removal system may conveniently be used to process plasma for analysis.

Furthermore, the sample may suitably comprise plasma proteins such as enriched plasma proteins. In this embodiment, plasma may be processed as described herein, and may then be subjected to size exclusion chromatography, buffer exchange, or other such treatments in order to arrive at a sample comprising the proteins from said plasma, which may offer advantages such as superior performance in analytical instruments.

Moreover, it is a specific advantage of embodiments of the invention when the sample is blood or a blood product that many of the biomarkers taught herein to be associated with acute brain injury such as stroke are amenable to detection or monitoring from blood from extant subjects for the first time; known techniques have relied on assay of cerebrospinal fluid, often from deceased subjects, and therefore have not previously amounted to a disclosure of aiding diagnosis in a living subject as is taught herein.

### Reference Standard

The reference standard typically refers to a sample from a healthy individual i.e. one who has not suffered acute brain damage, cerebrovascular accident or related injury.

The reference standard can an actual sample analysed in parallel. Alternatively the reference standard can be one or more values previously derived from a comparative sample e.g. a sample from a healthy subject. In such embodiments a mere numeric comparison may be made by comparing the value determined for the sample from the subject to the numeric value of a previously analysed reference sample. The advantage of this is not having to duplicate the analysis by determining concentrations in individual reference samples in parallel each time a sample from a subject is analysed.

Suitably the reference standard is matched to the subject being analysed e.g. by gender e.g. by age e.g. by ethnic background or other such criteria which are well known in the art. The reference standard may be a number such as an absolute concentration drawn up by one or more previous studies.

Reference standards may suitably be matched to specific patient sub-groups e.g. elderly subjects, or those with a previous relevant history such as a predisposition to stroke or having experienced one or more stroke(s) earlier in life.

Suitably the reference standard is matched to the sample type being analysed. For example the concentration of the biomarker polypeptide(s) being assayed may vary depending on the type or nature of the sample. It will be immediately apparent to the skilled worker that the concentration value(s) for the reference standard should be for the same or a comparable sample to that being tested in the method(s) of the invention. For example, if the sample being assayed is blood then the reference standard value should be for blood to ensure that it is capable of meaningful cross-comparison and therefore a meaningful quantitative ratio being calculated. In particular, extreme care must be taken if inferences are attempted by comparison between concentrations determined for a subject of interest and concentrations determined for reference standards where the nature of the sample is non-identical between the two. Suitably the sample type for the reference standard and the sample type for the subject of interest are the same.

It should be noted that for some embodiments of the invention, the polypeptide concentrations determined may be compared to a previous sample from the same subject. This can be beneficial in monitoring the progress of brain damage in a subject. This can be beneficial in monitoring the course and/or effectiveness of a treatment of a subject. In this embodiment the method may comprise further step(s) of comparing the quantitative ratio(s) determined for the sample of interest to one or more quantitative ratio(s) determined for the same polypeptide(s) from different samples such as samples taken at different time points for the same subject. By making such a comparison, information can be gathered about whether a particular polypeptide marker is increasing or decreasing in a particular subject. This information may be useful in diagnosing or predicting changes over time, or changes inhibited or stimulated by a particular treatment or therapy regime, or any other variable of interest. Thus if a polypeptide biomarker of acute brain damage is elevated, or elevated further, in a sample from a later time point from the same subject then this indicates a likelihood of brain damage progressing or worsening in said subject. Equally, if a polypeptide biomarker of acute brain damage is decreased in a sample from a later time point from the same subject then this indicates a likelihood of improvement or lessening of acute brain damage in said subject. Clearly if these effects are observed in a subject undergoing treatment for the brain damage, then corresponding inferences regarding the effectiveness of the treatment may equally be drawn according to the present invention. In other words, when a subject is undergoing treatment, if a polypeptide biomarker of acute brain damage is decreased in a sample from a later time point from the same subject then this indicates a likelihood that the treatment is effective; if a polypeptide biomarker of acute brain damage is elevated, or elevated further, in a sample from a later time point from the same subject then this indicates a likelihood that the treatment is ineffective.

In this way, the invention can be used to determine whether, for example after treatment of the patient with a drug or candidate drug, the disease has progressed or not, or that the rate of disease progression has been modified. The result can lead to a prognosis of the outcome of the disease.

### Combinations

The invention may be applied as part of a panel of biomarkers in order to provide a more robust diagnosis or prognosis. Moreover, the invention may be applied as part of a panel of biomarkers in order to provide a more complete picture of the disease state or possible outcomes for a given patient.

Of course, the skilled reader will appreciate that the specific biomarkers of the present invention may be advantageously combined with other markers known in the art. Such extended groups which comprise the specific biomarkers or panels of biomarkers discussed herein are of course intended to be embraced by the invention. Selection of further known markers for testing in such an embodiment may be accomplished by the skilled reader according to the appropriate sources. In this context additional biomarkers may relate to stroke, to other acute brain damage disorders from which a differential diagnosis of stroke is required, or to other diseases commonly associated with patients with stroke or whose symptoms mimic those of stroke.

Suitably said subject is a human.

Suitably said subject is a non-human mammal.

Suitably said subject is a rodent.

### Positional Information

Marker polypeptides of the present invention may show a gradient of concentration in microdialysis fluids directly related to their proximity to the site of brain injury or insult. It should be noted that for some embodiments of the invention, the polypeptide concentrations determined may be compared from different regions of the brain. In particular the polypeptide concentrations in a brain region immediately adjacent to the site of insult or injury may be compared to more distal regions within the same brain hemisphere and/or with the unaffected contralateral hemisphere.

More suitably where the type of brain injury is ischaemic stroke the adjacent region is the infarct core and the more distant region within the same hemisphere is the penumbra.

### Detection

A marker protein may have its expression modulated, i.e. quantitatively increased or decreased, in patients with acute brain damage such as stroke. The degree to which expression differs in normal versus affected states need only be large enough to be visualised via standard characterisation techniques, such as silver staining of 2D-electrophoretic gels, measurement of representative peptide ions using isobaric mass tagging and mass spectrometry or immunological detection methods including Western blotting, enzyme-linked immunosorbent assay (ELISA) or radioimmunoassay. Other such standard characterisation techniques by which expression differences may be visualised are well known to those skilled in the art. These include successive chromatographic separations of fractions and comparisons of the peaks, capillary electrophoresis, separations using micro-channel networks, including on a micro-chip, and mass spectrometry methods including multiple reaction monitoring (MRM) and TMTcalibrator (Dayon et al 2009).

The extent to which the protein level is modulated will typically vary in inverse relationship to the distance from the site of brain damage. In the case of brain microdialysates the modulations seen will be relatively large and typically a ratio >2 is indicative of a disease-related change in expression. In more distal sites such as cerebrospinal fluid and/or plasma the extent of modulation (changes in concentration of protein detected) may be lower than in brain microdialysates yet still provide diagnostically or prognostically useful information. In such materials (e.g. cerebrospinal fluid and/or plasma) typically a ratio >1.3 would be considered representative of brain damage.

Chromatographic separations can be carried out by high performance liquid chromatography as described in Pharmacia literature, the chromatogram being obtained in the form of a plot of absorbance of light at 280 nm against time of separation. The material giving incompletely resolved peaks is then re-chromatographed and so on.

Capillary electrophoresis is a technique described in many publications, for example in the literature “Total CE Solutions” supplied by Beckman with their P/ACE 5000 system. The technique depends on applying an electric potential across the sample contained in a small capillary tube. The tube has a charged surface, such as negatively charged silicate glass. Oppositely charged ions (in this instance, positive ions) are attracted to the surface and then migrate to the appropriate electrode of the same polarity as the surface (in this instance, the cathode). In this electroosmotic flow (EOF) of the sample, the positive ions move fastest, followed by uncharged material and negatively charged ions. Thus, proteins are separated essentially according to charge on them.

Micro-channel networks function somewhat like capillaries and can be formed by photoablation of a polymeric material. In this technique, a UV laser is used to generate high energy light pulses that are fired in bursts onto polymers having suitable UV absorption characteristics, for example polyethylene terephthalate or polycarbonate. The incident photons break chemical bonds with a confined space, leading to a rise in internal pressure, mini-explosions and ejection of the ablated material, leaving behind voids which form micro-channels. The micro-channel material achieves a separation based on EOF, as for capillary electrophoresis. It is adaptable to micro-chip form, each chip having its own sample injector, separation column and electrochemical detector: see J. S. Rossier et al., 1999, Electrophoresis 20: pages 727-731.

Other methods include performing a binding assay for the marker protein. Any reasonably specific binding agent can be used. Preferably the binding agent is labelled. Preferably the assay is an immunoassay, especially between the biomarker and an antibody that recognises the protein, especially a labelled antibody. It can be an antibody raised against part or all of the marker protein, for example a monoclonal antibody or a polyclonal anti-human antiserum of high specificity for the marker protein.

Where the binding assay is an immunoassay, it may be carried out by measuring the extent of the protein/antibody interaction. Any known method of immunoassay may be used. A sandwich assay is preferred. In an exemplary sandwich assay, a first antibody to the marker protein is bound to the solid phase such as a well of a plastics microtitre plate, and incubated with the sample and with a labelled second antibody specific to the protein to be assayed. Alternatively, an antibody capture assay can be used. Here, the test sample is allowed to bind to a solid phase, and the anti-marker protein antibody is then added and allowed to bind. After washing away unbound material, the amount of antibody bound to the solid phase is determined using a labelled second antibody, anti- to the first.

In another embodiment, a competition assay is performed between the sample and a labelled marker protein or a peptide derived therefrom, these two antigens being in competition for a limited amount of anti-marker protein antibody bound to a solid support. The labelled marker protein or peptide thereof can be pre-incubated with the antibody on the solid phase, whereby the marker protein in the sample displaces part of the marker protein or peptide thereof bound to the antibody.

In yet another embodiment, the two antigens are allowed to compete in a single co-incubation with the antibody. After removal of unbound antigen from the support by washing, the amount of label attached to the support is determined and the amount of protein in the sample is measured by reference to standard titration curves established previously.

The binding agent in the binding assay may be a labelled specific binding agent, which may be an antibody or other specific binding agent. The binding agent will usually be labelled itself, but alternatively it may be detected by a secondary reaction in which a signal is generated, e.g. from another labelled substance.

The label may be an enzyme. The substrate for the enzyme may be, for example, colour-forming, fluorescent or chemiluminescent.

An amplified form of assay may be used, whereby an enhanced “signal” is produced from a relatively low level of protein to be detected. One particular form of amplified immunoassay is enhanced chemiluminescent assay. Conveniently, the antibody is labelled with horseradish peroxidase, which participates in a chemiluminescent reaction with luminol, a peroxide substrate and a compound which enhances the intensity and duration of the emitted light, typically 4-iodophenol or 4-hydroxycinnamic acid.

Another form of amplified immunoassay is immuno-PCR. In this technique, the antibody is covalently linked to a molecule of arbitrary DNA comprising PCR primers, whereby the DNA with the antibody attached to it is amplified by the polymerase chain reaction. See E. R. Hendrickson et al., Nucleic Acids Research 23: 522-529 (1995). The signal is read out as before.

The time required for the assay may be reduced by use of a rapid microparticle-enhanced turbidimetric immunoassay such as the type embodied by M. Robers et al., “Development of a rapid microparticle-enhanced turbidimetric immunoassay for plasma fatty acid-binding protein, an early marker of acute myocardial infarction”, Clin. Chem. 1998; 44:1564-1567.

The full automation of any immunoassay contemplated in a widely used clinical chemistry analyser such as the COBAS™ MIRA Plus system from Hoffmann-La Roche, described by M. Robers et al. supra, or the AxSYM™ system from Abbott Laboratories, should be possible and applied for routine clinical diagnosis.

It is also contemplated within the invention to use (i) an antibody array or ‘chip’, or a bead suspension array capable of detecting one or more proteins that interact with that antibody.

An antibody chip, antibody array or antibody microarray is an array of unique addressable elements on a continuous solid surface whereby at each unique addressable element an antibody with defined specificity for an antigen is immobilised in a manner allowing its subsequent capture of the target antigen and subsequent detection of the extent of such binding. Each unique addressable element is spaced from all other unique addressable elements on the solid surface so that the binding and detection of specific antigens does not interfere with any adjacent such unique addressable element.

A “bead suspension array” is an aqueous suspension of one or more identifiably distinct particles whereby each particle contains coding features relating to its size and colour or fluorescent signature and to which all of the beads of a particular combination of such coding features is coated with an antibody with a defined specificity for an antigen in a manner allowing its subsequent capture of the target antigen and subsequent detection of the extent of such binding. Examples of such arrays can be found at www.luminexcorp.com where application of the xMAP® bead suspension array on the Luminex® 100™ System is described.

Alternatively, the diagnostic sample can be subjected to isobaric mass tagging and LC-MS/MS as described herein. An example of preferred ways of carrying out isobaric protein tagging are set out in the examples section of this application.

Isobaric protein tagging using tandem mass tags has been shown before to be able to determine relative proteins levels in a highly accurate manner (Thompson et al., 2003, Dayon et al., 2008). In addition, numerous reports have been published in the last few years using iTRAQ for protein tagging in various tissues and fluids (Aggarwal et al., 2006). Especially for the discovery of biomarkers in various conditions, iTRAQ has been proved to be a highly suitable tool and has been used in cancer (Maurya et al., 2007, Garbis et al., 2008, Matta et al., 2008, Ralhan et al., 2008) and diabetes research (Lu et al., 2008) as well as in the quest for biomarkers in neurodegenerative disorders (Abdi et al., 2006) albeit in CSF.

### Multiple Selected Reaction Monitoring (SRM or MRM)

MRM/SRM is the scan type with the highest duty cycle and is used for monitoring one or more specific ion transition(s) at high sensitivity. Here, Q1 is set on the specific parent m/z (Q1 is not scanning), the collision energy is set to produce the optimal diagnostic charged fragment of that parent ion, and Q3 is set to the specific m/z of that fragment. Only ions with this exact transition will be detected. Historically used to quantify small molecules such as drug metabolites, the same principle can be applied to peptides, either endogenous moieties or those produced from enzymatic digestion of proteins. Again historically experiments were performed using triple quadrupole mass spectrometers but the recent introduction of hybrid instrument designs, which combine quadrupoles with ion traps, enables similar and improved experiments to be undertaken. The 4000QTRAP instrument therefore allows peptide and biomolecule quantitation to be performed at very high specificity and sensitivity using Multiple Reaction Monitoring (MRM). This is largely due to the use of the LINAC® Collision Cell, which subsequently enables many MRM scans to be looped together into one experiment to detect the presence of many specific ions (up to 100 different ions) in a complex mixture. Consequently it is now feasible to measure and quantify multiple peptides from many proteins in a single chromatographic separation. The area under the MRM LC peak is used to quantitate the amount of the analyte present. In a typical quantitation experiment, a standard concentration curve is generated for the analyte of interest. When the unknown sample is then run under identical conditions, the concentration for the analyte in the unknown sample can be determined using the peak area and the standard concentration curve.

The diagnostic sample can be subjected to analysis by MRM on an ion-trap mass spectrometer. Based on the mass spectrometry profiles of the marker proteins described below single tryptic peptides with specific known mass and amino acid sequences are identified that possess good ionising characteristics. The mass spectrometer is then programmed to specifically survey for peptides of the specific mass and sequence and report their relative signal intensity. Using MRM it is possible to survey for up to 5, 10, 15, 20, 25, 30, 40, 50 or 100 different marker proteins in a single LC-MS run. The intensities of the MRM peptides of the specific biomarkers of the present invention in the diagnostic sample are compared with those found in samples from subjects without disease allowing the diagnosis or prognosis to be made.

The MRM assay can be made more truly quantitative by the use of internal reference standards consisting of synthetic absolute quantification (AQUA) peptides corresponding to the MRM peptide of the marker protein wherein one or more atoms have been substituted with a stable isotope such as carbon-13 or nitrogen-15 and wherein such substitutions cause the AQUA peptide to have a defined mass difference to the native, lighter form of the MRM peptide derived from the diagnostic sample. By comparing the relative ion intensity of the native MRM and AQUA peptides the true concentration of the parent protein in the diagnostic sample can thus be determined. General methods of absolute quantitation by such isotope dilution methods are provided in Gerber, Scott A, et al. “Absolute quantification of proteins and phosphoproteins from cell lysates by tandem MS” PNAS, Jun. 10, 2003. Vol 100. No 12. p 6940-6945.

In some cases, whilst it is desirable to use isotope-doped standards to provide absolute quantitation in an SRM experiment it is not possible to use the AQUA approach described above. In such cases it is possible to use a pair of isotopic mass tags i.e. two tags with identical chemical structure but different levels of isotopic substitutions giving each a unique mass. Using two forms of the Tandem Mass Tags@ (TMT®) that differ in mass by 5 Da it is possible to label standard synthetic reference SRM peptides with a light tag prior to mixing to form a universal reference for all targeted peptides in an assay. Each patient sample is then subjected to trypsin digestion and the resulting peptides labelled with the heavy TMT tag. An aliquot of the TMT-labelled reference peptides is then added to the sample to give a final concentration of reference peptides that is relevant to the target range to be measured in the patient sample. The spiked sample is then subjected to a standard isotope dilution SRM assay and the concentrations of the SRM peptides from the patient sample are calculated by comparing ion intensites of the heavy form against those of the known concentrations of the lighter form.

An alternative form of MS-based assay for the relative or absolute quantitation of regulated peptides identified as biomarker candidates is the TMTcalibrator method developed by Proteome Sciences plc, Known amounts of synthetic peptides representing tryptic fragments of the candidate biomarker(s) with good MS/MS behaviour are labelled with four of the six reagents of the TMT6 set of isobaric mass tags (TMT6-128 to TMT6-131) and mixed in certain ratios. This allows a multi-point calibration curve reflecting physiological and/or disease-modified concentrations to be designed and implemented quickly. Subsequently, a diagnostic sample taken from a patient suffering from or suspected of suffering from acute brain injury such as stroke is labelled with TMT6-126 and the calibration mix is added to the study sample. During MS/MS of individual peptides, the TMT6-reporter ions of the calibrant peptides are produced and used to establish a calibration curve. The absolute amount of the peptide in the study sample is then readily derived by reading the TMT6126 ion intensity against the calibration curve. Further information on TMTcalibrator assays can be obtained from the Proteome Sciences website (www.proteomics.com).

A preferred method of diagnosis comprises performing a binding assay for the marker protein. Any reasonably specific binding partner can be used. Preferably the binding partner is labelled. Preferably the assay is an immunoassay, especially between the marker and an antibody that recognises the protein, especially a labelled antibody. It can be an antibody raised against part or all of it, most preferably a monoclonal antibody or a polyclonal anti-human antiserum of high specificity for the marker protein.

Thus, the marker proteins described above are useful for the purpose of raising antibodies thereto which can be used to detect the increased or decreased concentration of the marker proteins present in a diagnostic sample. Such antibodies can be raised by any of the methods well known in the immunodiagnostics field.

The antibodies may be anti- to any biologically relevant state of the protein. Thus, for example, they can be raised against the unglycosylated form of a protein which exists in the body in a glycosylated form, against a more mature form of a precursor protein, e.g. minus its signal sequence, or against a peptide carrying a relevant epitope of the marker protein.

The sample can be taken from any valid body tissue, especially body fluid, of a mammalian or non-mammalian subject, but preferably blood, plasma, serum or urine. Other usable body fluids include cerebrospinal fluid (CSF), semen and tears. Preferably the subject is a mammalian species such as a mouse, rat, guinea pig, dog or primate. Most preferably the subject is human.

The preferred immunoassay is carried out by measuring the extent of the protein/antibody interaction. Any known method of immunoassay may be used. A sandwich assay is preferred. In this method, a first antibody to the marker protein is bound to the solid phase such as a well of a plastic microtitre plate, and incubated with the sample and with a labelled second antibody specific to the protein to be assayed. Alternatively, an antibody capture assay can be used. Here, the test sample is allowed to bind to a solid phase, and the anti-marker protein antibody is then added and allowed to bind. After washing away unbound material, the amount of antibody bound to the solid phase is determined using a labelled second antibody, anti- to the first.

In another embodiment, a competition assay is performed between the sample and a labelled marker protein or a peptide derived therefrom, these two antigens being in competition for a limited amount of anti-marker protein antibody bound to a solid support. The labelled marker protein or peptide thereof can be pre-incubated with the antibody on the solid phase, whereby the marker protein in the sample displaces part of the marker protein or peptide thereof bound to the antibody.

In yet another embodiment, the two antigens are allowed to compete in a single co-incubation with the antibody. After removal of unbound antigen from the support by washing, the amount of label attached to the support is determined and the amount of protein in the sample is measured by reference to standard titration curves established previously.

The label is preferably an enzyme. The substrate for the enzyme may be, for example, colour-forming, fluorescent or chemiluminescent.

The binding partner in the binding assay is preferably a labelled specific binding partner, but not necessarily an antibody. The binding partner will usually be labelled itself, but alternatively it may be detected by a secondary reaction in which a signal is generated, e.g. from another labelled substance.

It is highly preferable to use an amplified form of assay, whereby an enhanced “signal” is produced from a relatively low level of protein to be detected. One particular form of amplified immunoassay is enhanced chemiluminescent assay. Conveniently, the antibody is labelled with horseradish peroxidase, which participates in a chemiluminescent reaction with luminol, a peroxide substrate and a compound which enhances the intensity and duration of the emitted light, typically 4-iodophenol or 4-hydroxycinnamic acid.

The use of a rapid microparticle-enhanced turbidimetric immunoassay such as the type embodied by M. Robers et al., “Development of a rapid microparticle-enhanced turbidimetric immunoassay for plasma fatty acid-binding protein, an early marker of acute myocardial infarction”, Clin. Chem. 1998; 44:1564-1567, significantly decreases the time of the assay. Thus, the full automation of any immunoassay contemplated in a widely used clinical chemistry analyser such as the COBAS™ MIRA Plus system from Hoffmann-La Roche, described by M. Robers et al. supra, or the AxSYM™ system from Abbott Laboratories, should be possible and applied for routine clinical diagnosis.

Alternatively, the diagnostic sample can be subjected to two dimensional gel electrophoresis to yield a stained gel in which the position of the marker proteins is known and the relative intensity of staining at the appropriate spots on the gel can be determined by densitometry and compared with a corresponding control or comparative gel.

In a yet further embodiment the diagnostic sample can be subjected to analysis by a mass-spectrometer-based assay such as multiple reaction monitoring (MRM) on a triple quadrupole mass spectrometer or on certain types of ion-trap mass spectrometer. For each differentially expressed protein it is possible to identify a set of tryptic peptides with specific known mass (parent mass) and amino acid sequence and which upon fragmentation release fragments of specific mass (fragment mass) that are unique to each protein. The detection of a fragment mass from a defined parent mass ion is known as a transition.

Identification of such proteotypic peptides can be made based on the mass spectrometry profiles of the differentially expressed proteins seen during biomarker discovery, or may be designed in silico using predictive algorithms known to the skilled practitioner. The mass spectrometer is then programmed to specifically survey only for the specific parent mass and fragment mass transitions selected for each protein and reports their relative signal intensity. Using MRM it is possible to survey for up to 5, 10, 15, 20, 25, 30, 40, 50 or 100 different marker proteins in a single LC-MS run. The relative abundances of the proteotypic peptides for each marker protein in the diagnostic sample are compared with those found in samples from subjects without acute brain injury such as stroke allowing the diagnosis to be made. Alternatively comparison may be made with levels of the proteins from earlier samples from the same patient thus allowing prognostic assessment of the stage and/or rate of progression of acute brain injury such as stroke in said patient.

In a further embodiment of the invention the MRM assay can be made more truly quantitative by the use of internal reference standards consisting of synthetic absolute quantification (AQUA) peptides corresponding to the proteotypic peptide of the marker protein wherein one or more atoms have been substituted with a stable isotope such as carbon-13 or nitrogen-15 and wherein such substitutions cause the AQUA peptide to have a defined mass difference to the native proteotypic peptide derived from the diagnostic sample. Once AQUA peptides equivalent to each proteotypic peptide from the differentially expressed biomarkers have been produced, they can be mixed to form a reference standard that is then spiked into the tryptic digest of the patient sample. The combined sample is then subjected to a programmed mass spectrometer-based assay where the intensity of the required transitions from the native and AQUA peptides is detected. By comparing the relative ion intensity of the native peptides from the sample and the spiked AQUA reference peptides the true concentration of the parent protein in the diagnostic sample can thus be determined. General methods of absolute quantitation are provided in Gerber, Scott A, et al. “Absolute quantification of proteins and phosphoproteins from cell lysates by tandem MS” PNAS, Jun. 10, 2003. Vol 100. No 12. p 6940-6945 which is incorporated herein by reference.

In a yet further embodiment of the invention an absolute quantitation can be made by using a TMT-SRM assay. Standard synthetic reference SRM peptides corresponding to the prototypic peptide of the marker protein are labelled with a light TMT tag having no isotope substitutions (light tag) prior to mixing to form a universal reference for all marker proteins in an assay. Each patient sample is then subjected to trypsin digestion and the resulting peptides labelled with the TMT tag having five isotopic substitution (heavy tag). An aliquot of the light TMT-labelled reference peptides is then added to the heavy TMT-labelled sample to give a final concentration of reference peptides that is relevant to the target range to be measured in the patient sample. The spiked sample is then subjected to a standard isotope dilution SRM assay and the concentrations of the SRM peptides from the patient sample are calculated by comparing ion intensities of the heavy form against those of the known concentrations of the lighter form.

The invention further includes the use for a diagnostic (and thus possibly prognostic) or therapeutic purpose of a partner material which recognises, binds to or has affinity for a marker protein specified above. Thus, for example, antibodies to the marker proteins, appropriately humanised where necessary, may be used in treatment. The partner material will usually be an antibody and used in any assay-compatible format, conveniently an immobilised format, e.g. as beads or a chip. Either the partner material will be labelled or it will be capable of interacting with a label.

The invention further includes a kit for use in a method of diagnosis and prognostic monitoring of acute brain injury such as stroke, which comprises a partner material, as described above, in an assay-compatible format, as described above, for interaction with a marker protein present in the diagnostic sample.

It is further contemplated within the invention to use (i) an antibody chip or array of chips, or a bead suspension array capable of detecting one or more proteins differentially expressed in acute brain injury such as stroke.

The method may further comprise determining an effective therapy for treating acute brain injury such as stroke.

In a further aspect, the present invention provides a method of treatment by the use of an agent that will restore the expression of one or more differentially expressed proteins in the acute brain injury such as stroke state towards that found in the normal state in order to prevent the development or progression of acute brain injury such as stroke. Preferably, the expression of the protein is restored to that of the normal state.

In a further aspect, the present invention provides a method whereby the pattern of differentially expressed proteins in a tissue sample or body fluid sample of an individual with acute brain injury such as stroke is used to predict the most appropriate and effective therapy to alleviate the acute brain injury such as stroke.

Also provided is a method of screening an agent to determine its usefulness in treating acute brain injury such as stroke, the method comprising:

(a) obtaining a sample of relevant tissue taken from, or representative of, a subject having acute brain injury such as stroke symptoms, who or which has been treated with the agent being screened;
(b) determining the presence, absence or degree of expression of the differentially expressed protein or proteins in the tissue from, or representative of, the treated subject; and,
(c) selecting or rejecting the agent according to the extent to which it changes the expression, activity or amount of the differentially expressed protein or proteins in the treated subject having acute brain injury such as stroke symptoms.

Preferably, the agent is selected if it converts the expression of the differentially expressed protein towards that of a normal subject. More preferably, the agent is selected if it converts the expression of the protein or proteins to that of the normal subject.

Also provided is a method of screening an agent to determine its usefulness in treating acute brain injury such as stroke, the method comprising:

(a) obtaining over time samples of relevant tissue or body fluid taken from, or representative of, a subject having acute brain injury such as stroke symptoms, who or which has been treated with the agent being screened;
(b) determining the presence, absence or degree of expression of a differentially expressed protein or proteins in said samples; and,
(c) determining whether the agent affects the change over time in the expression of the differentially expression protein in the treated subject having acute brain injury such as stroke symptoms.

Samples taken over time may be taken at intervals of weeks, months or years. For example, samples may be taken at monthly, two-monthly, three-monthly, four-monthly, six-monthly, eight-monthly or twelve-monthly intervals.

A change in expression over time may be an increase or decrease in expression, compared to the initial level of expression in samples from the subject and/or compared to the level of expression in samples from normal subjects. The agent is selected if it slows or stops the change of expression over time.

In the screening methods described above, subjects having differential levels of protein expression comprise:

(a) normal subjects and subjects having acute brain injury such as stroke; and,
(b) subjects having acute brain injury such as stroke symptoms which have not been treated with the agent and subjects having acute brain injury such as stroke which have been treated with the agent.

### Diagnosis and Prognosis

The term “diagnosis”, as used herein, includes the provision of any information concerning the existence, non-existence or probability of acute brain injury such as stroke in a patient. It further includes the provision of information concerning the type or classification of the disorder or of symptoms which are or may be experienced in connection with it. It encompasses prognosis of the medical course of the condition. It further encompasses information concerning the age of onset.

### Treatment

It will be understood that where treatment is concerned, treatment includes any measure taken by the physician to alleviate the effect of acute brain injury such as stroke on a patient. Thus, although reversal of the damage or elimination of the damage or effects of acute brain injury such as stroke is a desirable goal, effective treatment will also include any measures capable of achieving reduction in the degree of damage or severity of the effects or progression.

In one aspect, the invention provides a method of treatment by the use of an agent that will restore the expression of one or more differentially expressed proteins in the acute brain injury such as stroke state towards that found in the normal state in order to prevent the development or progression of acute brain injury such as stroke. Preferably, the expression of the protein is restored to that of the normal state.

In a further aspect, the present invention provides a method whereby the pattern of differentially expressed proteins in a sample from an individual with acute brain injury such as stroke is used to predict the most appropriate and effective therapy to alleviate the neurological damage.

### Antibodies

Antibodies against the marker proteins disclosed herein can be produced using known methods. These methods of producing antibodies include immunising a mammal (e.g. mouse, rat, rabbit, horse, goat, sheep or monkey) with the protein. Antibodies may be obtained from immunised animals using any of a variety of techniques known in the art, and screened, preferably using binding of antibody to antigen of interest. Isolation of antibodies and/or antibody-producing cells from an animal may be accompanied by a step of sacrificing the animal.

As an alternative or supplement to immunising a mammal with a protein, an antibody specific for the protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with the protein, or may be one constructed using sequences obtained from an organism which has been exposed to the antigen of interest.

The antibodies may bind or be raised against any biologically relevant state of the protein. Thus, for example, they can be raised against the unglycosylated form of a protein which exists in the body in a glycosylated form, against a more mature form of a precursor protein, e.g. minus its signal sequence, or against a peptide carrying a relevant epitope of the marker protein.

Antibodies may be polyclonal or monoclonal, and may be multispecific (including bispecific), chimeric or humanised antibodies. Antibodies according to the present invention may be modified in a number of ways. Indeed the term “antibody” should be construed as covering any binding substance having a binding domain with the required specificity. Thus, the invention covers antibody fragments, derivatives, functional equivalents and homologues of antibodies, including synthetic molecules and molecules whose shape mimics that of an antibody enabling it to bind an antigen or epitope.

Examples of antibody fragments, capable of binding an antigen or other binding partner, are the Fab fragment consisting of the VL, VH, C1 and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

Antibody fragments, which recognise specific epitopes, may be generated by known techniques. For example, such fragments include, but are not limited to, the F(ab′)2 fragments which can be produced by pepsin digestion of the antibody molecule and the Fab fragments which can be generated by reducing the disulfide bridges of the F(ab′)2 fragments. Alternative, Fab expression libraries may be constructed (Huse, et al., 1989, Science 246: 1275-1281) to allow rapid and easy identification of monoclonal Fab fragments with the desired specificity.

The term “monoclonal antibody” as used herein refers to an antibody obtained from a substantially homogenous population of antibodies, i.e. the individual antibodies comprising the population are identical apart from possible naturally occurring mutations that may be present in minor amounts. Monoclonal antibodies can be produced by the method first described by Kohler and Milstein, Nature, 256:495, 1975 or may be made by recombinant methods, see Cabilly et al, U.S. Pat. No. 4,816,567, or Mage and Lamoyi in Monoclonal Antibody Production Techniques and Applications, pages 79-97, Marcel Dekker Inc, New York, 1987.

In the hybridoma method, a mouse or other appropriate host animal is immunised with the antigen by subcutaneous, intraperitoneal, or intramuscular routes to elicit lymphocytes that produce or are capable of producing antibodies that will specifically bind to the nanoparticles used for immunisation. Alternatively, lymphocytes may be immunised in vitro. Lymphocytes then are fused with myeloma cells using a suitable fusing agent, such as polyethylene glycol, to form a hybridoma cell, see Goding, Monoclonal Antibodies: Principles and Practice, pp. 59-103 (Academic Press, 1986).

The hybridoma cells thus prepared can be seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high level expression of antibody by the selected antibody producing cells, and are sensitive to a medium such as HAT medium.

Culture medium in which hybridoma cells are growing is assayed for production of monoclonal antibodies directed against the protein. Preferably, the binding specificity is determined by enzyme-linked immunoabsorbance assay (ELISA). The monoclonal antibodies of the invention are those that specifically bind to the protein.

In a preferred embodiment of the invention, the monoclonal antibody will have an affinity which is greater than micromolar or greater affinity (i.e. an affinity greater than 10-6 mol) as determined, for example, by Scatchard analysis, see Munson & Pollard, Anal. Biochem., 107:220, 1980.

After hybridoma cells are identified that produce neutralising antibodies of the desired specificity and affinity, the clones can be subcloned by limiting dilution procedures and grown by standard methods. Suitable culture media for this purpose include Dulbecco's Modified Eagle's Medium or RPM1-1640 medium. In addition, the hybridoma cells may be grown in vivo as ascites tumours in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Nucleic acid encoding the monoclonal antibodies of the invention is readily isolated and sequenced using procedures well known in the art, e.g. by using oligonucleotide probes that are capable of binding specifically to genes encoding the heavy and light chains of murine antibodies. The hybridoma cells of the invention are a preferred source of nucleic acid encoding the antibodies or fragments thereof. Once isolated, the nucleic acid is ligated into expression or cloning vectors, which are then transfected into host cells, which can be cultured so that the monoclonal antibodies are produced in the recombinant host cell culture.

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies, humanised antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP 0 184 187 A, GB 2 188 638 A or EP 0 239 400 A. Cloning and expression of chimeric antibodies are described in EP 0 120 694 A and EP 0 125 023 A.

An antibody against a marker protein described herein will bind to said protein. Preferably, said antibody specifically binds said protein. By “specific” is meant that the antibody binds to said protein with an affinity significantly higher than it displays for other molecules.

The term “antibody” includes polyclonal antiserum, monoclonal antibodies, fragments of antibodies such as single chain and Fab fragments, and genetically engineered antibodies. The antibodies may be chimeric or of a single species.

The term “marker protein” or “biomarker” includes all biologically relevant forms of the protein identified, including post-translational modification. For example, the marker protein can be present in the body tissue in a glycosylated, phosphorylated, multimeric or precursor form.

The term “control” refers to a normal human subject, i.e. one not suffering from acute brain injury such as stroke.

The terminology “increased/decreased concentration . . . compared with a control sample” does not imply that a step of comparing is actually undertaken, since in many cases it will be obvious to the skilled practitioner that the concentration is abnormally high or low. Further, when the stages of acute brain injury such as stroke are being monitored progressively, or when a course of treatment is being monitored, the comparison made can be with the concentration previously seen in the same subject at an earlier stage of progression of the disease, or at an earlier stage of treatment or before treatment has commenced.

The term “valid body tissue” or “relevant tissue” means any tissue in which it may reasonably be expected that a marker protein would accumulate in relation to acute brain injury such as stroke. It may be a cerebrospinal fluid sample or a sample of blood or a blood derivative such as plasma or serum.

The term “antibody array” or “antibody microarray” means an array of unique addressable elements on a continuous solid surface whereby at each unique addressable element an antibody with defined specificity for an antigen is immobilised in a manner allowing its subsequent capture of the target antigen and subsequent detection of the extent of such binding. Each unique addressable element is spaced from all other unique addressable elements on the solid surface so that the binding and detection of specific antigens does not interfere with any adjacent such unique addressable element.

The term “bead suspension array” means an aqueous suspension of one or more identifiably distinct particles whereby each particle contains coding features relating to its size and colour or fluorescent signature and to which all of the beads of a particular combination of such coding teatures is coated with an antibody with a defined specificity for an antigen in a manner allowing its subsequent capture of the target antigen and subsequent detection of the extent of such binding. Examples of such arrays can be found at www.luminexcorp.com where application of the xMAP® bead suspension array on the Luminex® 100™ System is described.

Mass spectrometry assay” means any quantitative method of mass spectrometery including but not limited to selected reaction monitoring (SRM), multiple reaction monitoring (MRM), absolute quantitation using isotopedoped peptides (AQUA), Tandem Mass Tags with SRM (TMTSRM) and TMTcalibrator.

The term ‘mutant’ of a biomarker such as a polypeptide biomarker of the invention should have its normal meaning in the art. Mutants are sometimes referred to as ‘variants’ or ‘alleles’. The key is to detect biomarkers as have been set out herein. The biomarkers may possess individual variations in the form of mutations or allelic variants between individuals being studied. Therefore there may be some degree of deviation from the exemplary SEQ ID NOs provided herein. The SEQ ID NOs provided herein are to assist the skilled reader in identifying and working with the polypeptides/biomarkers of the invention and are not intended as a restricted and inflexible definition of the individual polypeptides being assayed. Thus minor sequence differences between the SEQ ID NOs provided and the actual sequences of the polypeptide biomarkers being detected will be expected within the boundaries of normal variation between subjects. This should not affect the working of the invention.

The term ‘comprises’ (comprise, comprising) should be understood to have its normal meaning in the art, i.e. that the stated feature or group of features is included, but that the term does not exclude any other stated feature or group of features from also being present.

### Fragments/Peptides

It will be appreciated by the skilled worker that the details of the biomarkers discussed herein and in particular the sequences presented for them are given to facilitate their detection. The important information being gathered is the presence or absence (or particular level) of the biomarker in the sample being studied. There is no particular requirement that the full length polypeptide be scored. Indeed, via many of the suitable mass spectrometry based modes of detection set out herein, detection takes place by assaying particular fragments of the polypeptide of interest being present which are thus taken to indicate the presence of the overall biomarker polypeptide in the sample. Therefore the invention embraces the detection of fragments of the polypeptide biomarkers. Moreover, the kits and peptides of the invention may comprise fragments of the polypeptides and need not comprise the full length sequences exemplified herein. Suitably the fragment is sufficiently long to enable its unique identification by mass spectrometry.

Thus a fragment is suitably at least 6 amino acids in length, suitably at least 7 amino acids in length, suitably at least 8 amino acids in length, suitably at least 9 amino acids in length, suitably at least 10 amino acids in length, suitably at least 15 amino acids, suitably at least 25 amino acids, suitably at least 50 amino acids, suitably at least 100 amino acids, or suitably the majority of the biomarker polypeptide of interest. Suitably a fragment comprises a small fragment of the biomarker polypeptide of interest, whilst being long enough to retain an identifiable mass.

For any given polypeptide or set of polypeptides being detected by mass spectrometry based assay, the assay may be conducted via MRM techniques mentioned herein. In this embodiment, certain unique peptides and in particular certain transitions are especially advantageous to detect the peptides of interest. These are typically selected to give the highest representation (or combinations may be used such as any or all peptides giving a particular level of representation if multiple fragments/transitions give similar levels). Especially preferred transitions used for monitoring are those mentioned in the accompanying examples and/or figures.

### Sequence Homology/Identity

Although sequence homology can also be considered in terms of functional similarity (i.e., amino acid residues having similar chemical properties/functions), in the context of the present document it is preferred to express homology in terms of sequence identity. Sequence comparisons can be conducted by eye or, more usually, with the aid of readily available sequence comparison programs. These publicly and commercially available computer programs can calculate percent homology (such as percent identity) between two or more sequences.

Percent identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues (for example less than 50 contiguous amino acids). For comparison over longer sequences, gap scoring is used to produce an optimal alignment to accurately reflect identity levels in related sequences having insertion (s) or deletion (s) relative to one another. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package, FASTA (Altschul et al., 1990, J. Mol. Biol. 215:403-410) and the GENEWORKS suite of comparison tools.

In the context of the present document, a homologous amino acid sequence is taken to include an amino acid sequence which is at least 40, 50, 60, 70, 80 or 90% identical. Most suitably a polypeptide having at least 90% sequence identity to the biomarker of interest will be taken as indicative of the presence of that biomarker; more suitably a polypeptide which is 95% or more suitably 98% identical at the amino acid level will be taken to indicate presence of that biomarker. Suitably said comparison is made over at least the length of the polypeptide or fragment which is being assayed to determine the presence or absence of the biomarker of interest. Most suitably the comparison is made across the full length of the polypeptide of interest. The same considerations apply to nucleic acid nucleotide sequences.

### Alternate Methods

It will be understood by the skilled reader that specific techniques exemplified herein may be varied if desired using readily available alternatives to achieve the same effect. For example, assay of the biomarker levels in a blood sample may be carried out by western blot or by isobaric protein tagging or by ELISA or by any other suitable means known in the art.

### Quantitative Ratios

It will be appreciated that there are a number of biomarkers disclosed herein which are significantly decreased in subjects having suffered acute brain damage such as stroke. These are scientifically equally valid as is discussed in the accompanying examples section. However, in practical terms it is more technically challenging to determine an absence or decrease in a particular biomarker in a sample being analysed. In particular it is difficult to control for the genuine detection of a decreased amount of a marker versus a problem in detection. For this reason, in preferred embodiments of the invention the biomarkers used are those which are elevated or increased in acute brain damage such as stroke. These have the advantage that positive identification of the biomarker(s) of interest can positively aid diagnosis.

Thus is should be noted that the quantitative ratios determined herein describe the ratio of the concentration in the sample of the subject being analysed to the concentration in the reference standard. Thus a ratio of 1.3 is achieved when the concentration in the sample is 1.3 times the concentration in the reference standard. Clearly the ratios could be expressed in another manner (e.g. in reverse) but for consistency the ratios are discussed herein as sample:standard such that a ratio of 1.3 means a concentration in the sample being 30% greater than that of the concentration in the standard.

### Biomarkers

There are advantages to using more than one biomarker in the methods of the invention. The advantages include increased specificity and/or sensitivity to the methods of the invention. We present panels of biomarkers which are particularly advantageous in the method(s) of the invention.

GSTP-1 and Peroxiredoxins 1 & 6 represent useful markers for management of stroke. For the reasons noted above, we also present larger panels of proteins. These panels have technical advantages such as further improving diagnostic sensitivity and/or specificity. Certain panels disclosed also have the advantage of providing prognostic information. Accordingly the inventors performed a review of literature relating to stroke and cardiovascular biomarkers and pathway analysis for all 53 proteins found differentially expressed in infarct and penumbra compared to contralateral brain microdialysates. Following this comprehensive bioinformatic approach three groups of biomarkers were selected, Panel A, Panel B and Panel C. These are shown below in descending priority order.

| PANEL A | ID | Description |
|---|---|---|
| N^{o}1 | ACBP_HUMAN | Acyl-CoA-binding protein |
| N^{o}2 | CSRP1_HUMAN | Cysteine and glycine-rich protein 1 |
| N^{o}3 | PEBP1_HUMAN | Phosphatidylethanolamine-binding |
| | | protein 1 |
| N^{o}4 | DDAH1_HUMAN | N(G),N(G)-dimethylarginine |
| | | dimethylaminohydrolase 1 |
| N^{o}5 | MT3_HUMAN | Metallothionein-3 (MT-3) |
| N^{o}6 | CYTB_HUMAN | Cystatin-B |

| PANEL B | ID | Description |
|---|---|---|
| N^{o}1 | PPIA_HUMAN | Peptidyl-prolyl cis-trans isomerase A |
| N^{o}2 | NFM_HUMAN | Neurofilament medium polypeptide |
| N^{o}3 | UBIQ_HUMAN | Ubiquitin. |
| N^{o}4 | B2MG_HUMAN | Beta-2-microglobulin precursor |
| N^{o}5 | CYTC_HUMAN | Cystatin-C precursor (Cystatin-3) |
| N^{o}6 | SH3L1_HUMAN | SH3 domain-binding glutamic acid-rich- |
| | | like protein. |
| N^{o}7 | TPIS_HUMAN | Triosephosphate isomerase |
| N^{o}8 | MBP_HUMAN | Myelin basic protein (MBP) |
| N^{o}9 | MT2_HUMAN | Metallothionein-2 (MT-2) |

| PANEL C | ID | Description |
|---|---|---|
| N^{o}1 | NFM_HUMAN | Neurofilament medium polypeptide |
| N^{o}2 | COTL1_HUMAN | Coactosin-like protein. |
| N^{o}3 | THY1_HUMAN | Thy-1 membrane glycoprotein precursor |
| N^{o}4 | PROF1_HUMAN | Profilin-1 |
| N^{o}5 | TYB4_HUMAN | Thymosin beta-4 |
| N^{o}6 | MT1E_HUMAN | Metallothionein-1E |
| N^{o}7 | FABPB_HUMAN | Fatty acid-binding protein, brain (B-FABP) |
| N^{o}8 | GFAP_HUMAN | Glial fibrillary acidic protein (GFAP). |
| N^{o}9 | CAH2_HUMAN | Carbonic anhydrase 2 |
| N^{o}10 | CERU_HUMAN | Ceruloplasmin precursor |
| N^{o}11 | DCD_HUMAN | Dermcidin precursor |
| N^{o}12 | DEF1_HUMAN | Neutrophil defensin 1 precursor (HNP-1 |

In some embodiments, Panels A, B and C may be considered as a single cohesive group of biomarkers which may be referred to as the enlarged panel ABC.

In addition to the defined panels A-C larger panels of biomarker proteins can be used in the method of the invention.

Panel 1

| Biomarker Polypeptide | Further Details |
|---|---|
| Acyl-CoA-binding protein | IC v CT |
| Apolipoprotein A-II precursor | Panel 1A |
| Apolipoprotein A-IV precursor | |
| Carbonic anhydrase 1 | |
| Carbonic anhydrase 2 | |
| Chitinase-3-like protein 1 precursor | |
| Cofilin-1 | |
| Cystatin-B | |
| Fibrinogen alpha chain precursor | |
| Flavin reductase | |
| Glial fibrillary acidic protein | |
| Hemoglobin subunit alpha | |
| Histone H1.2 | |
| Histone H1.5 | |
| Lysozyme C precursor | |
| N(G),N(G)-dimethylarginine | |
| dimethylaminohydrolase 1 | |
| Neurofilament medium polypeptide | |
| Neutrophil defensin 1 precursor | |
| Peptidyl-prolyl cis-trans isomerase A | |
| Phosphatidylethanolamine-binding | |
| protein 1 | |
| Thymosin beta-10 | |
| Thymosin beta-4 | |
| Triosephosphate isomerase | |
| Tropomyosin alpha-3 chain | |
| Acyl-CoA-binding protein | IC v P |
| Beta-2-microglobulin precursor | Panel 1B |
| Coactosin-like protein | |
| Complement C4-A precursor | |
| Cystatin-B | |
| Cysteine and glycine-rich protein 1 | |
| Fatty acid-binding protein, brain | |
| Fibrinogen alpha chain precursor | |
| Glutathione S-transferase P | |
| Heterogeneous nuclear | |
| ribonucleoprotein G | |
| Metallothionein-3 | |
| Myelin basic protein [ISOFORM 3] | |
| Neutrophil defensin 1 precursor | |
| Paralemmin | |
| Peptidyl-prolyl cis-trans isomerase A | |
| Peroxiredoxin-2 | |
| Peroxiredoxin-6 | |
| Phosphatidylethanolamine-binding | |
| protein 1 | |
| Plasma retinol-binding protein | |
| precursor | |
| Plasminogen precursor | |
| Platelet basic protein precursor | |
| Profilin-1 | |
| SH3 domain-binding glutamic acid- | |
| rich-like protein | |
| Thioredoxin | |
| Ubiquitin | |
| Aquaporin-4 | P v CT |
| Coactosin-like protein | Panel 1C |
| Cystatin-B | |
| Cysteine and glycine-rich protein 1 | |
| Diazepam binding inhibitor, splice form | |
| 1c | |
| Fibrinogen alpha chain precursor | |
| Glial fibrillary acidic protein | |
| Hydroxyacylglutathione hydrolase | |
| Kininogen-1 precursor | |
| Lysozyme C precursor | |
| Metallothionein-2 | |
| Metallothionein-3 | |
| Myoglobin | |
| N(G),N(G)-dimethylarginine | |
| dimethylaminohydrolase 1 | |
| Neurofilament medium polypeptide | |
| Peptidyl-prolyl cis-trans isomerase A | |
| Phosphatidylethanolamine-binding | |
| protein 1 | |
| Plasminogen precursor | |
| Platelet basic protein precursor | |
| Profilin-1 | |
| Prothrombin precursor | |
| SH3 domain-binding glutamic acid-rich- | |
| like protein | |
| Spectrin beta chain, brain 1 | |
| Stathmin | |
| Ubiquitin | |
| Ubiquitin carboxyl-terminal hydrolase | |
| isozyme L1 | |

An advantage of the markers in Panel 1 is that they are all increased in an affected subject. In other words, an increase in the level of such biomarker(s) is indicative of an increased likelihood of acute brain damage. This facilitates positive detection and helps to eliminate potential problems arising from false negatives due to technical problems of detection being mistaken for an indication that particular biomarker is decreased in a subject. In particular, the markers in Panel 1 share the advantage that the quantitative ratio for said polypeptides is each above 1.3. This is evidenced in the examples section. This has the advantage of providing statistically significant confidence in each marker used from this panel in a method according to the present invention.

Panel 1 also defines subgroups of markers according to the particular type of analysis in which their statistically significant increased expression was detected. Thus the designations “IC vs CT”, “IC vs P” and “P vs CT” in the ‘further details’ column provide three further sub-groups of markers:

### Panel 1A— “IC vs CT”

### Panel 1B— “IC vs P”

### Panel 1C— “P vs CT”

Panel 1 also defines subgroups of markers which are found to be elevated to a statistically significant level in more than one type of analysis. Thus, individual biomarkers shown to be underlined are shown to occur at elevated levels in affected subjects in at least two of the three types of analysis undertaken (“IC vs CT”, “IC vs P” and “P vs CT”). Moreover, there are a smaller number of markers which are shown to occur at elevated levels in affected subjects in all three of the three types of analysis undertaken (“IC vs CT” and “IC vs P” and “P vs CT”). These may be easily identified by comparing the underlined biomarkers in the three treatments and noting those which occur in each of those three treatments in Panel 1 above. Thus, four further subgroups of marker are defined ([Panel 1D— “IC vs CT” and “IC vs P”]; [Panel 1E “IC vs CT” and “P vs CT”]; [Panel 1F “IC vs P” and “P vs CT”]; [Panel 1G “IC vs CT” and “IC vs P” and “P vs CT”]).

Panel 1 also defines a further subgroup which can be described as “X vs CT” where X is P or IC. In other words, this subgroup comprises any marker which is in either IC vs CT (Panel 1A) or P vs CT (Panel 1C) (or both). Thus Panel 1H is defined as “any vs CT”. This has the advantage of collating all markers which show an increase in an affected sample compared to the control.

Panel 2

| | | | |
|---|---|---|---|
| Acyl-CoA-binding | Apolipoprotein A- | Apolipoprotein A-IV | Aquaporin-4 |
| protein | II precursor | precursor | |
| Beta-2-microglobulin | Carbonic | Chitinase-3-like | Coactosin-like |
| precursor | anhydrase 2 | protein 1 precursor | protein |
| Cofilin-1 | Complement C4- | Cystatin-B | Cysteine and |
| | A precursor | | glycine-rich protein |
| | | | 1 |
| Diazepam binding | Fibrinogen alpha | Flavin reductase | Hemoglobin |
| inhibitor, splice form | chain precursor | | subunit alpha |
| 1c | | | |
| Heterogeneous | Histone H1.2 | Histone H1.5 | Hydroxyacylglutathione |
| nuclear | | | hydrolase |
| ribonucleoprotein G | | | |
| Kininogen-1 | Lysozyme C | Metallothionein-2 | Metallothionein-3 |
| precursor | precursor | | |
| Myelin basic protein | Myoglobin | N(G),N(G)- | Neurofilament |
| [ISOFORM 3] | | dimethylarginine | medium |
| | | dimethylaminohydrolase | polypeptide |
| | | 1 | |
| Neutrophil defensin 1 | Paralemmin | Peptidyl-prolyl cis- | Peroxiredoxin-2 |
| precursor | | trans isomerase A | |
| Phosphatidylethanol | Plasma retinol- | Plasminogen | Platelet basic |
| amine-binding | binding protein | precursor | protein precursor |
| protein 1 | precursor | | |
| Profilin-1 | Prothrombin | SH3 domain-binding | Stathmin |
| | precursor | glutamic acid-rich- | |
| | | like protein | |
| Thymosin beta-10 | Thymosin beta-4 | Triosephosphate | Ubiquitin |
| | | isomerase | |
| Ubiquitin carboxyl- | | | |
| terminal hydrolase | | | |
| isozyme L1 | | | |

Panel 2 presents biomarker polypeptides which are disclosed herein for the first time to have a connection to any kind of brain damage, particularly to acute brain damage such as stroke. Thus it is an advantage of individual markers of panel 2 that they are disclosed for the first time in connection with brain damage.

Panel 2A

| | | | |
|---|---|---|---|
| Acyl-CoA-binding | Coactosin-like | Cystatin-B | Cysteine and glycine-rich |
| protein | protein | | protein 1 |
| Fibrinogen alpha | Lysozyme C | Metallothionein-3 | N(G),N(G)- |
| chain precursor | precursor | | dimethylarginine |
| | | | dimethylaminohydrolase 1 |
| Neurofilament | Neutrophil | Peptidyl-prolyl cis- | Phosphatidylethanolamine- |
| medium | defensin 1 | trans isomerase A | binding protein 1 |
| polypeptide | precursor | | |
| Plasminogen | Platelet basic | Profilin-1 | SH3 domain-binding |
| precursor | protein | | glutamic acid-rich-like |
| | precursor | | protein |
| Ubiquitin | | | |

Panel 2A biomarkers are a sub-group of Panel 2 and have the further property that they are increased in at least two out of the three microdialysis studies (IC:P, IC:CT and P:CT) presented in the examples section, suggesting an association with the site of brain damage.

Panel 2B

| | | | |
|---|---|---|---|
| Cystatin-B | Fibrinogen | Peptidyl-prolyl cis- | Phosphatidylethanolamine- |
| | alpha chain | trans isomerase A | binding protein 1 |
| | precursor | | |

Panel 2B biomarkers are a sub-group of Panel 2A and have the further property that they are increased in each of the three microdialysis studies (IC:P, IC:CT and P:CT) presented in the examples section, representing a close association with the site of brain damage.

Numerous markers are demonstrated herein such as in the examples section. Some markers show strong associations in more than one patient/experiment in the tables of data and figures. Those markers showing associations for two or more patients/exp.'s in herein are preferred.

References to Metallothionein-1E (MT1E_HUMAN) suitably refer to the protein having the sequence of accession number P04732.

### DEFINITIONS

The term ‘comprises’ (comprise, comprising) should be understood to have its normal meaning in the art, i.e. that the stated feature or group of features is included, but that the term does not exclude any other stated feature or group of features from also being present.

The following abbreviations may be used herein: 1-D PAGE, one-dimensional polyacrylamide gel electrophoresis; CT, contralateral; CSF, cerebrospinal fluid; ECF, extracellular fluid; ELISA, enzyme-linked immunosorbent assay; GSTP1; glutathione S-transferase P; IC, infarct core; HUG, Geneva University Hospitals; IEF, isoelectric focusing; LACB, β-lactoglobulin; MALDI, matrix-assisted laser desorption ionization; MCA, middle cerebral artery; MS, mass spectrometry; MS/MS, tandem mass spectrometry; PRDX, peroxiredoxin; P, penumbra; RP-LC, reversed-phase liquid chromatography; SAH, subarachnoid hemorrhage; S100B, protein S100-B; TBI, traumatic brain injury; TMT, tandem mass tag; TMT2, duplex TMT; TMT6, sixplex TMT; TOF/TOF, tandem time-of-flight.

Exploring brain microdialysates of stroke patients with ms/ms-based quantitative proteomics is described.

### BRIEF DESCRIPTION OF THE FIGURES

FIG. 1: Gel images of 4 of the microdialysis samples under study (i.e., CTd, ICd, Pe, and CTe) after separation with 1-D PAGE (home-made 15% Tris-glycine gels) and silver staining. Ten μL of each microdialysate was loaded on the gels.

FIG. 2: Immunoblot validation of increased level of GSTP1 in IC with respect to CT microdialysates. Pooled microdialysis samples (n=3; i.e., ICa-c and CTd-f; 1.5 μg) were separated with 1-D PAGE (home-made 15% Tris-glycine gel). The recombinant GSTP1 (31.25 ng) and post-mortem CSF (10 μL) were taken as a positive control whereas ante-mortem CSF (20 μL) served as a negative control.

FIG. 3: ELISA measurement of proteins GSTP1 (a), PRDX1 (b), and S100B (c) in the sera of control and stroke patients.

FIG. 4 shows Distribution of relative abundance of TMT2 reporter-ions for Expa-f before and after final normalization steps. Basically, a translation was operated on the relative abundances for both data for reporter-ions at m/z=126.1 (distribution in red) and 127.1 (distribution in green) in order that the common area between both distribution was maximal (i.e., to minimize the quantitative differences between both populations).

FIG. 5 shows Silver-stained 1-D PAGE images of microdialysis samples relative to Expa-c, and Expf. Ten μL of each microdialysate was loaded on home-made 15% Tris-glycine gels. These images were used for designing the TMT2-based quantitative study.

FIG. 6 shows Experimental evaluation of the cut-off values to set for the TMT2-based quantitative experiments of the human brain microdialysates. Following the experimental procedure detailed in the article, TMT6 were used to tag identical samples of IC, P, and CT microdialysates. Because no difference was expected between identical samples (e.g., the two IC samples), deviations from 1:1 ratio were evaluated in term of false positive. The mean cut-off values were averaged from the IC, P, and CT results. To have symmetrical cut-off values at 1% FDR, 1.68 and 0.59 (instead of 0.61) cut-off ratios had to be chosen.

FIG. 7 shows bar charts of progression of the levels of several proteins in the MDs. The displayed results correspond to the proteins reported in the corresponding tables for which an evolution could be determined from the ratios obtained by MS.

FIG. 8 shows a bar chart of the evolution of the levels of PRDX1 and PRDX6 in the MDs; these trends were determined from the ratios obtained by MS.

FIG. 9 shows a diagram of proteomic quantitative workflow used for the analysis of human brain MDs of stroke patients.

FIG. 10 shows an example of tandem mass spectrum (a) and tandem mass spectrum zoomed on the TMT reporter-ion area (b) obtained when comparing IC and P MDs.

FIG. 11 shows iSRM chromatogram of DENATLDGGDVLFTGR peptide (DDAH1 protein) in human plasma digested with trypsin.

FIG. 12 shows iSRM chromatogram of TPEEYPESAK peptide (DDAH1 protein) in human plasma digested with trypsin.

FIG. 13 shows iSRM chromatogram of SQVVAGTNYFIK peptide (CYTB protein) in human plasma digested with trypsin.

FIG. 14 shows iSRM chromatogram of GYGYGQGAGTLSTDK peptide (CSRP1 protein) in human plasma digested with trypsin.

FIG. 15 shows iSRM chromatogram of GLESTTLADK peptide (CSRP1 protein) in human plasma digested with trypsin.

FIG. 16 shows iSRM chromatogram of LYEQLSGK peptide (PEBP1 protein) in human plasma digested with trypsin.

The invention is now described by way of example. These examples are intended to be illustrative, and are not intended to limit the appended claims.

### EXAMPLES

### Summary of Examples

In vivo human cerebral microdialysis fluids of stroke patients were investigated for the discovery of potential protein biomarkers associated with cerebrovascular disorders. Microdialysates from the infarct core (IC), the penumbra (P) and the unaffected contralateral (CT) brain regions of patients suffering an ischemic stroke were compared qualitatively and quantitatively using a shotgun proteomic approach. The changes in protein amounts were assessed in several cases; e.g., IC vs. P (n=2), IC vs. CT (n=2), and P vs. CT (n=2). Tandem mass tags (TMTs) were used to label the content of microdialysis fluids after reduction, alkylation and digestion with trypsin. After TMT labeling, the pooled samples were fractionated with off-gel electrophoresis and the resulting fractions were analyzed with RP-LC MALDI TOF/TOF. One hundred and fifty six proteins were identified in the whole brain microdialysates. MS/MS quantitative analysis showed 43 proteins with increased amounts in the IC with respect to the P and CT samples. Twenty six proteins were increased in the P with respect to the CT. Glutathione S-transferase P (GSTP1), peroxiredoxin-1 (PRDX1) and protein S100-B (S100B) changes were validated with immunoblot on pooled microdialysis samples and/or ELISA on blood of unrelated control and stroke patients (n=28). In conclusion, the correlation between proteomic quantitative data of the human brain microdialysis and early validations on blood samples from stroke patients demonstrate the value of the methods and biomarker panels described herein.

### Experimental Procedures

### Materials

β-Lactoglobulin (LACB) from bovine milk (˜90%), trypsin from porcine pancreas, iodoacetamide (IAA, ≧99%), recombinant GSTP1 (from human, expressed in Escherichia coli), tris(2-carboxyethyl)phosphine hydrochloride (TCEP) 0.5 M, and a-cyano-4-hydroxycinnamic acid were purchased from Sigma (St. Louis, Mo., USA). Triethylammonium hydrogen carbonate buffer (TEAB) 1 M pH=8.5, sodium dodecyl sulphate (SDS, ≧98%), and trifluoroacetic acid (TFA, ≧99.5%) were from Fluka (Büchs, Switzerland). Hydroxylamine solution 50 wt. % in H2O (99.999%) was from Aldrich (Milwaukee, Wis., USA). Hydrochloric acid (25%) and ammonium dihydrogen phosphate ((NH4)H2PO4) were from Merck (Darmstadt, Germany). Water for chromatography LiChrosolv® and acetonitrile Chromasolv® for HPLC (≧99.9%) were respectively from Merck and Sigma-Aldrich (Büchs, Switzerland). Duplex and sixplex TMTs (TMT2 and TMT6) were provided by Proteome Sciences (Frankfurt am Main, Germany). Oasis® HLB 1 cc (10 and 30 mg) extraction cartridges were from Waters (Milford, Mass., USA). Immobiline™ DryStryp pH 3-10, 13 cm and IPG buffer pH 3-10 were from GE Healthcare (Uppsala, Sweden). Glycerol 50% and mineral oil were from Agilent Technologies (Wilmington, Del., USA).

### Sample Collection

### Microdialysates

Patients with a massive, so-called malignant infarction in the middle cerebral artery (MCA) territory were treated in the Neurointensive Care Unit of Vail d'Hebron University Hospital according to an institutional protocol which combines induced moderate hypothermia (32.5° C.) with decompressive craniotomy. Six malignant MCA infarction patients were included (mean age 50±9.3 years; malignant MCA infarction side: 2 lefts, 4 rights; sex: 4 females, 2 males).

Malignant MCA infraction patients were monitored with high-cut-off (100 kDa) cerebral microdialysis catheters (CMA-71, CMA Microdialysis, Stockholm, Sweden) which were inserted at different brain regions (CT, P, and IC). Computed tomography scan was used to confirm brain microdialysis catheter location. Microdialysate samples were obtained hourly for 5 days after perfusion with an artificial CSF solution (i.e., NaCl 147 mM, KCl 2.7 mM, CaCl2 1.2 mM, and MgCl2 0.85 mM) by using a CMA 106 micropump (CMA microdialysis).

Prior to freezing and storage at −80° C. a routine analysis for glucose, lactate, pyruvate and glycerol/glutamate/urea concentrations in microdialysis samples was performed with the CMA600 analyser (CMA microdialysis). Proteomic analysis was performed on pooled brain microdialysates obtained during the first 24 h of brain monitoring. Table 1 summarizes the patients, the different brain regions sampled and the experimental labels that were used.

TABLE 1
Overview of the brain microdialysis samples under study.

| | | Infarct Core | Penumbra | Contralateral |
|---|---|---|---|---|
| Experiment | Patient | (IC) | (P) | (CT) |
| Expₐ | Patient a | ICₐ | Pₐ | |
| Exp_{b} | Patient b | IC_{b} | P_{b} | |
| Exp_{c} | Patient c | IC_{c} | | CT_{c} |
| Exp_{d} | Patient d | IC_{d} | | CT_{d} |
| Expₑ | Patient e | | Pₑ | CTₑ |
| Exp_{f} | Patient f | | P_{f} | CT_{f} |

### CSF Samples

Ante- and post-mortem CSF collection and clinical data of deceased and living patients have been reported previously (15). Briefly, control ante-mortem CSF samples were collected by routine diagnostic lumbar puncture from living healthy patients. Post-mortem CSF samples were collected by ventricular puncture at autopsy.

### Blood Samples

The blood samples of control and stroke patients were collected between October 2005 and January 2008 at the Geneva University Hospitals (HUG). During this period, all patients exhibiting unambiguous symptoms and signs of an acute or sub-acute stroke, who were hospitalized at the HUG, were enrolled in the study. Exclusion criteria were defined as follows: (1) a stroke onset time superior to 3 days or occurring after a previous stroke in the preceding 3 months; (2) extra-cerebral hemorrhage or trauma, such SAH, subdural hematoma or traumatic brain injury (TBI); (3) presence of other, potentially confounding pathologies such as cancer, kidney or liver failure, myocardial infarction, and psychiatric conditions. Each patient included in the study underwent a standardized protocol of clinical and neuroradiological assessments, and therapeutic interventions that was supervised by trained neurologists from the Department of Neurology of the HUG.

Controls were defined as patient's family relatives, as patients suffering from various types of medical and surgical conditions or even from non-cerebrovascular neurological conditions. They were required not to have a past or present history of stroke, cerebrovascular, or thrombotic diseases.

Blood samples were collected according to Standard Operating Procedures (SOP) described by the SOP Internal Working Group (16). Briefly, blood samples were drawn into red top blood collection tubes (silica coated tubes, 6 mL, 13* 100 mm, ref 368815, BD vacutainers, Plymouth, UK) and kept at room temperature during 45 min to allow the clot to form. No additive (anti-coagulant, protease inhibitor or preservative) was used. At the end of the clotting time, samples were centrifuged (1000×g for 10 min at room temperature) to discard the cell pellet. Immediately after, each serum sample was aliquoted and stored at −80° C. until use. For the studies reported here, 14 controls and 14 stroke patients, age and gender matched were randomly selected among all the participants collected. Table 2 summarizes the characteristics of the stroke patients and controls.

TABLE 2
General characteristics of the studied population (blood samples).

| | Controls | | Stroke | |
|---|---|---|---|---|
| n | 14 | | 14 | |
| Age (years) | | | | |
| Mean ± standard deviation | 69.3 ± 14.5 | | 69.5 ± 15.6 | |
| Median (minimum-maximum) | 72.5 | (40-88) | 72.0 | (39-89) |
| Gender | | | | |
| Female n (%) | 5 | (25.7) | 5 | (25.7) |
| Male n (%) | 9 | (64.3) | 9 | (64.3) |

| Time onset of symptoms (min) | | |
|---|---|---|
| Mean ± standard deviation | 486.1 ± 542.8 | |
| Median (minimum-maximum) | 150 | (90-1440) |

The local ethical committees approved these studies, and written consent was obtained from patients (or relatives) in accordance with the Helsinki declaration.

### SDS Page

Ten μL of brain microdialysates was separated with one-dimensional (1-D) SDS polyacrylamide gel electrophoresis (PAGE) on a home-made 15% Tris-glycine gel (8×7×0.1 cm). Twenty μL of ante- and post-mortem CSF samples (respective concentrations of 172 and 359 μg mL-1 was determined with the Bradford assay (17)) were also loaded, and taken as controls. Gels were stained with silver nitrate (18). The gel images were analyzed with the ImageQuant TL software (GE Healthcare). Signal of each lane was integrated and relatively quantified with respect to the other lane signals obtained on the same gel.

### Reduction, Alkylation, Digestion, and TMT Labeling

Appropriate volumes of microdialysis samples were taken according to the 1-D PAGE analyses, in order to compare equal protein amounts (i.e., weights) in each quantitative experiment (i.e., Expa-f in Table 1). LACB was spiked in equal quantity in each sample pairs at 1/50 of the expected protein amount (i.e., weight). The 6×2 samples were dried.

The samples were dissolved in 100 μL of TEAB 100 mM adjusted to pH=8 with diluted HCl. One μL of SDS 1% and 2 μL TCEP 50 mM were added to each tube. The reduction was carried out at 60° C. for 1 h. Alkylation was performed (addition of 1 μL of IAA 400 mM) during 30 min in the dark. Ten μL trypsin 0.2 μg·μL−1 freshly prepared in the TEAB solution was added. The digestion was carried out overnight at 37° C. TMT2 labeling was achieved for 1 h, after addition of 40.3 μL of TMT2 reagent in CH3CN (i.e. 0.83 mg, 2.42×10-6 mol). The tags were used as described in Table 3.

TABLE 3
Overview of TMT² experiments.

| | Reporter | Reporter | LACB (reporter | LACB (reporter |
|---|---|---|---|---|
| Experiment | 126.1 | 127.1 | 126.1)^{ç} | 127.1)^{ç} |
| Expₐ | Pₐ | ICₐ | 0.58 ± 0.04 | 0.42 ± 0.04 |
| Exp_{b} | IC_{b} | P_{b} | 0.45 ± 0.07 | 0.55 ± 0.07 |
| Exp_{c} | CT_{c} | IC_{c} | 0.51 ± 0.07 | 0.49 ± 0.07 |
| Exp_{d} | IC_{d} | CT_{d} | 0.45 ± 0.06 | 0.55 ± 0.06 |
| Expₑ | CTₑ | Pₑ | 0.54 ± 0.04 | 0.46 ± 0.04 |
| Exp_{f} | P_{f} | CT_{f} | 0.47 ± 0.08 | 0.53 ± 0.08 |

^{ç}Normalized mean abundance; the isotopic correction was done. These data were used for subsequent normalization to reduce the manipulation bias.

The quantities of peptides to label varied from a microdialysate sample pair to another because of the different available protein amounts. These quantities were estimated to range from 1.5 to 27 μg according to the used microdialysate volumes and the estimated concentrations determined with respect to ante-mortem CSF (see above). Eight μL of hydroxylamine 5% was added for 15 min reaction. The differentially TMT2-labeled samples were pooled in a new tube. The pooled samples were dried. TMT6 experiments were carried out with the same protocol.

### Off-Gel Electrophoresis

The samples were desalted with Oasis® HLB 1 cc (30 mg) extraction cartridges. After drying, the samples were dissolved in 1616.4 μL H2O with 172.8 μL glycerol 50% and 10.8 μL of carrier ampholytes IPG buffer pH 3-10. The IPG strips (pH 3-10, 13 cm) were assembled on the off-gel trays and rehydrated for 30 min with a solution of 89.8% H2O, 9.6% glycerol 50%, and 0.6% of carrier ampholytes. The samples were loaded on the 12 off-gel wells. The isoelectric focusing (IEF) separations were carried out using the 3100 OFFGEL Fractionator (Agilent Technologies) with a limiting current of 50 μA, and a limit of 20 kV·h before holding the voltage to 500 V. The fractions were collected and their pH was measured (744 pH Meter and Biotrode from Metrohm (Herisau, Switzerland)). The fractions were dried, cleaned with Oasis® HLB 1 cc (10 mg) extraction cartridges, and dried again.

### RP-LC MALDI TOF/TOF

Matrix-assisted laser desorption ionization (MALDI) tandem time-of-flight (TOF/TOF) MS was performed on a 4800 Proteomics Analyzer from Applied Biosystems (Foster City, Calif., USA). The off-gel fractions were first separated with reversed-phase liquid chromatography (RP-LC) using an Alliance system from Waters equipped with a flow splitter. A home-packed 5 μm 200 Å Magic C18 AQ 0.1×100 mm column was used. The separation was run for 60 min using a gradient of H₂O/CH3CN/TFA 97%/3%/0.1% (solvent A) and H₂O/CH₃CN/TFA 5%/95%/0.1% (solvent B). The gradient was run as follows: 0-10 min 98% A and 2% B, then to 90% A and 10% B at 12 min, 50% A and 50% B at 55 min, and 98% B at 60 min at a flow rate estimated to 400 nL·min-1. One minute fractions were deposited onto the MALDI plates using a home-made LC-robot. The matrix (a-cyano-4-hydroxycinnamic acid in H2O/CH3CN/TFA 50%/50%/0.1% with 10 mM NH4H2PO4) was then spotted onto the plates. All mass spectra were acquired in positive-ionization mode with an m/z scan range of 800-4000 (1000 shoots with laser intensity of 4000 a.u.). After selection of 20 most-intense precursors at the maximum, MS/MS experiments (1500 shoots with laser intensity of 4500 a.u.) were performed at medium collision energy.

### Protein Identification and Quantitation

Peak lists were generated using the 4000 Series Explorer software from Applied Biosystems. For each sample, the mgf files resulting from the analysis of the 12 off-gel fractions were combined and searched against UniProt-Swiss-Prot/TrEMBL database (12.6_{—}4 Dec. 2007, 5610855 protein entries) using Phenyx 2.6 (GeneBio, Geneva, Switzerland). Homo sapiens taxonomy (93005 protein entries) (and separately Bos taurus (17268 protein entries) to search for the spiked LACB) was specified for database searching. Variable amino acid modifications were oxidized methionine.

TMT2-labeled peptide amino terminus and TMT2-labeled lysine (+225.1558 Da) were set as fixed modifications, as well as carbamidomethylation of cysteines. When using TMT6, a mass increment of +229.1629 Da was specified for TMT6-labeled peptide amino termini and TMT6-labeled lysines. Trypsin was selected as the enzyme, with one potential missed cleavage, and the normal cleavage mode was used. Only one search round was used with selection of “turbo” scoring. The peptide p value was 1 E-6 for all runs. The AC and peptide scores were set to control the peptide false peptide discovery rate below 1% (the scores varied from 7.0 to 7.5). The parent ion tolerance was 1.1 Da. Only proteins matching two different peptide sequences were selected and extracted into an excel file using the dedicated Phenyx export. Further filters were applied. Only proteins identified with two different unique peptides were finally kept. When a mass spectrum was attributed to several peptide sequences, all the matched peptides were removed.

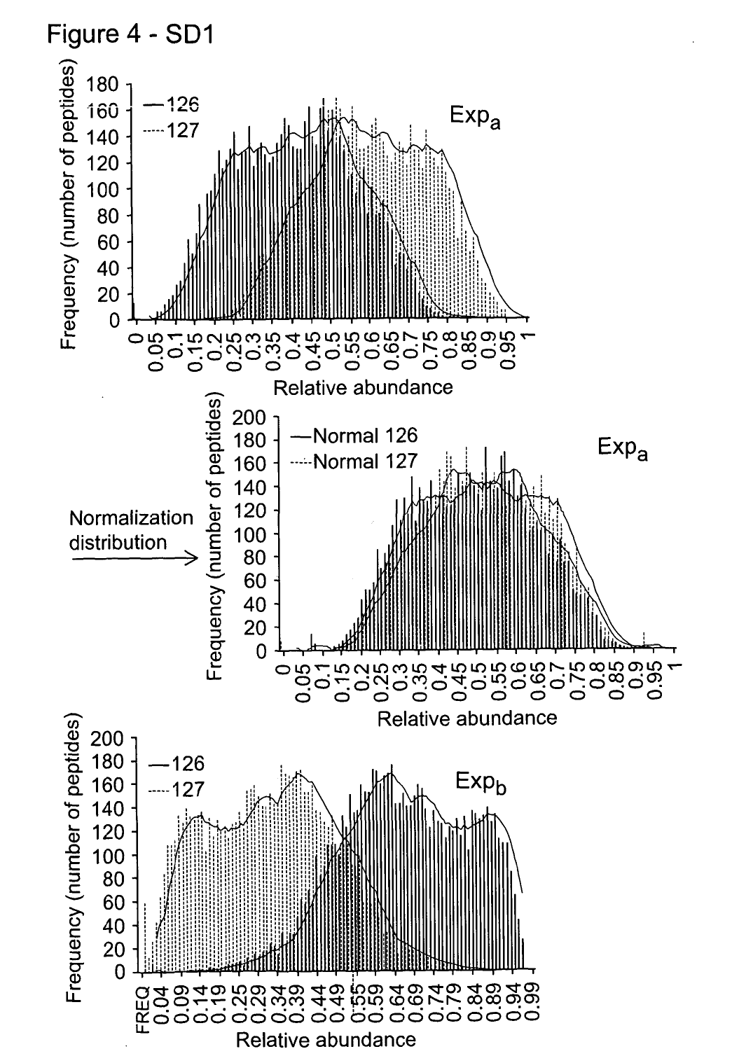

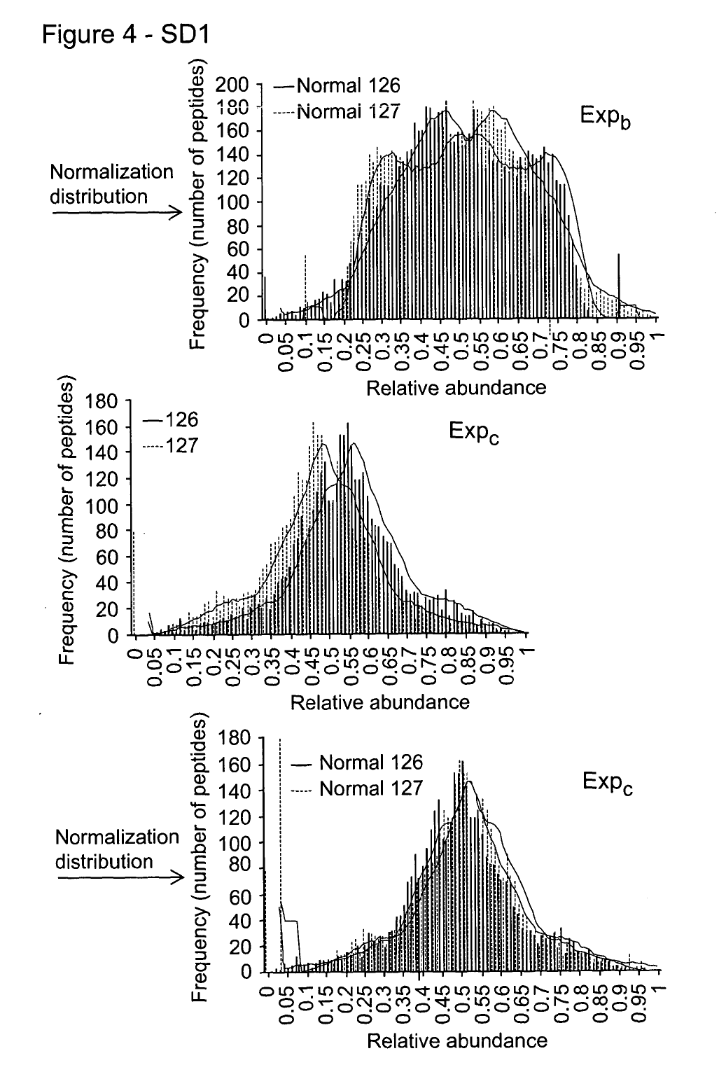

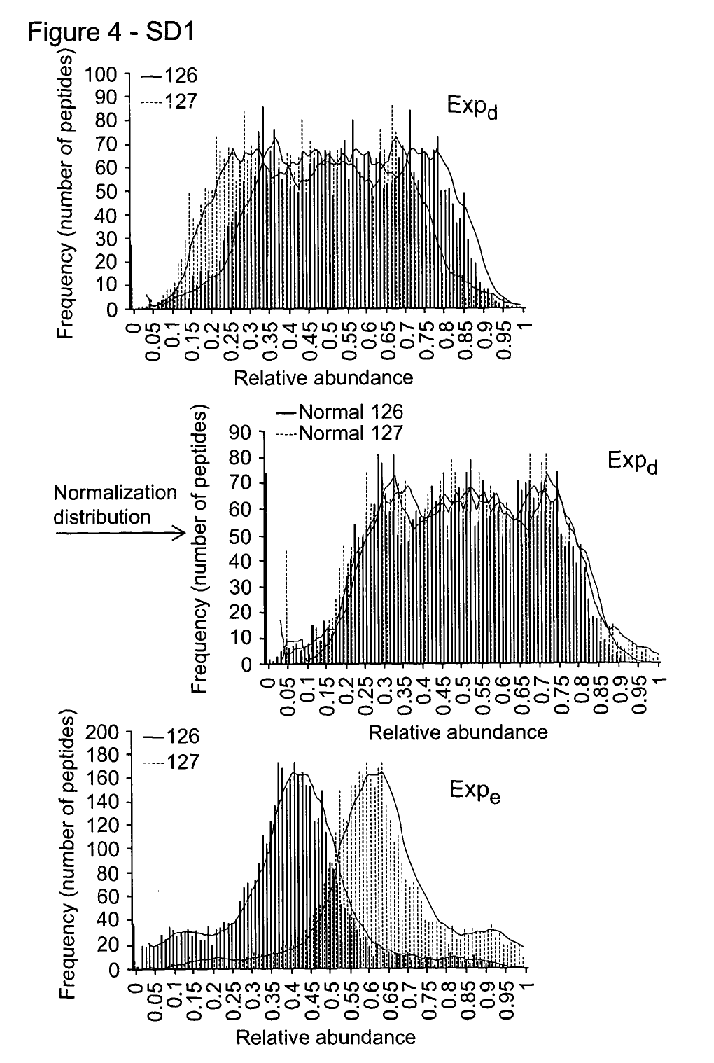

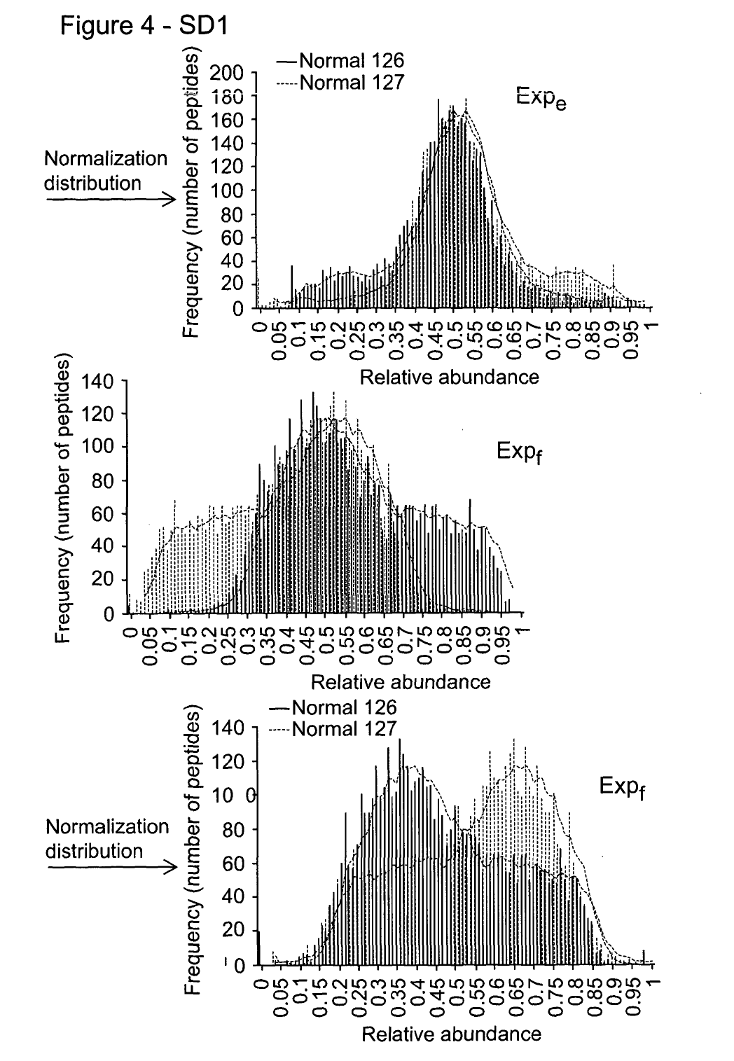

The areas of the reporter-ions were extracted from the tandem mass spectra using the analysis tool of the 4000 Series Explorer software. Quantitation was carried out only with peptides which were unique to a protein; at least two peptides with different sequences were needed to quantify a protein. The processing of the data was carried out as already described (13). The processing included an isotopic correction and a normalization with the spiked LACB standard. For each peptide, the relative abundance of each reporter-ion was calculated as the ratio of the reporter-ion abundance by the sum of all reporter-ion abundances. The protein ratios were then calculated as the ratios of the arithmetic averages of their peptide relative abundances (corresponding to each reporter-ion channel), according to the Libra module used in the Trans-Proteomic Pipeline. A final normalization step was performed assuming that most peptides were in equal quantities in the compared samples; i.e., the common areas between the relative abundance frequency distributions of both TMT2-labeled groups had to be maximal (shown in FIG. 4). The normalization coefficients were obtained on the entire reporter-ion dataset (i.e., even when peptides were not matched to any sequences).

Quantitative cut-off values were determined by comparison of identical microdialysis samples analyzed with the protocol described previously. Basically, TMT6 reagents were used to tag identical samples of IC, P, and CT microdialysates. Because no difference was expected between identical samples (e.g., the two IC samples), deviations from 1:1 ratio were considered as falsely positive. The relative abundances provided in each TMT channel were mixed randomly. Ratios were then calculated between identical samples, and geometrical means were obtained from clusters of 10 ratio data points. These mean ratios were then used to evaluate the cut-off values at a given false positive rate. The final cut-off values were averaged from the IC, P, and CT results.

### Immunoblot Analysis of Pooled IC and CT Microdialysates

One and a half μg of pooled IC (n=3; i.e., ICa-c) and 1.5 μg of pooled CT microdialysates (n=3; i.e., CTd-f) were separated with 1-D SDS PAGE. Twenty μL of ante-mortem CSF, 10 μL of post-mortem CSF, and 31.25 ng of recombinant GSTP1 were also separated. Separated proteins were electroblotted onto a nitrocellulose membrane as described by Towbin et al. (19). Membranes were incubated 1 h with 5% milk-PBS-Tween 0.05% for blocking. Immunodetection was performed with the anti-human GSTP rabbit polyclonal antibody (MBL International Corp., Woburn, Mass., USA) diluted 1/2000 in 1% milk-PBS-Tween 0.05%. After several washing steps, appropriate secondary antibody HRP (Dako, Glostrup, Denmark) was incubated 1 h at 1/2000. ECL plus Western Blotting detection system Kit (GE Healthcare) was used for detection. The membrane was finally scanned with the Typhoon 9400 (GE Healthcare).

### ELISA

S100B and PRDX1 were validated using commercial enzyme-linked immunosorbent assay (ELISA) kits from Abnova Corp, (Taipei city, Taiwan) and Biovendor GmbH (Heidelberg, Germany), respectively, according to manufacturer's recommendations. Concerning GSTP1, as no commercial assay is currently available, a sandwich immunoassay was developed in house and used as previously described (20, 21). Statistical analyses and graphs were performed using GraphPad Prism software (version 4.03, GraphPad software Inc., San Diego, Calif., USA).

### Example 1

### Microdialysis Analysis

Tandem mass tags (TMTs) (13, 14) were used herein to compare brain microdialysis samples of ischemic stroke patients. TMTs comprise a set of isobaric labels. These isobaric labels are synthesized with heavy and light isotopes to present the same total mass but to provide reporter-ions at different masses after activation with collision-induced dissociation and subsequent tandem mass spectrometry (MS/MS). The reporter-ion abundances are used to perform relative quantitation of the peptides labeled with different versions of the TMTs, and by extension determine relative protein amounts.

Samples from the infarct core (IC), the penumbra (P), and the contralateral (CT) brain regions of patients suffering a stroke were investigated. This proteomic study highlighted 43 proteins with increased amount in the IC with respect to the P and the CT microdialysates. Twenty six proteins were increased in the P compared to the CT samples. As candidate markers, glutathione S-transferase P (GSTP1), peroxiredoxin-1 (PRDX1), and S100B were further assessed with immunoassays on microdialysis samples and/or blood of stroke patients that finally confirmed their increased levels in stroke cases.

The human brain microdialysates were sampled in pairs from 2 brain regions of six stroke patients. Six quantitative MS/MS-based comparisons with TMT2, with reporter-ions at m/z=126.1 and 127.1, were carried out in experiences Expa-f (Table 3).

### Concentration of IC, P and CT Microdialysis Samples

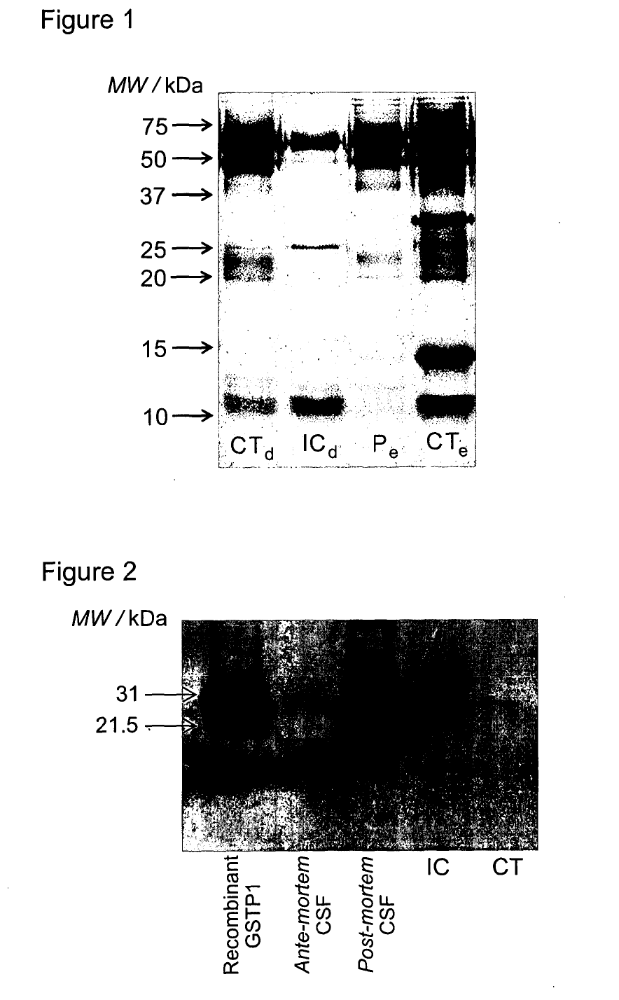

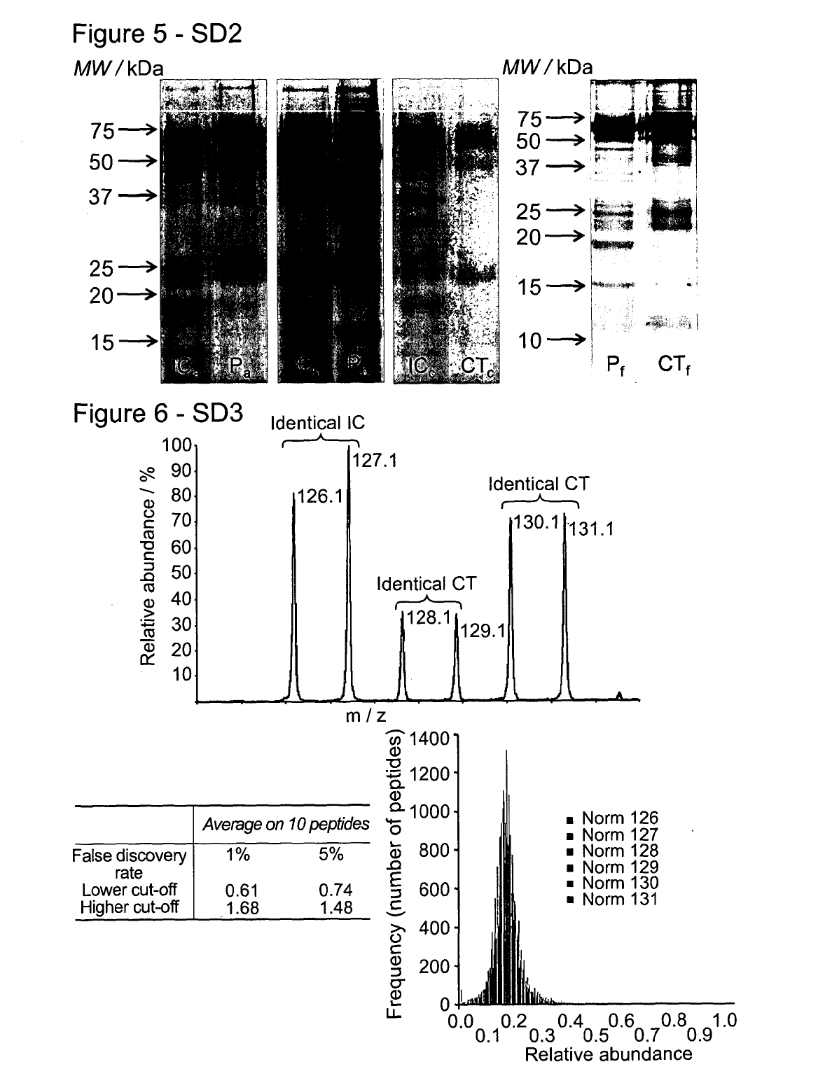

In FIG. 1 are displayed brain microdialysates ICd, CTd, Pe, and CTe separated with 1-D PAGE (1-D PAGE images of others samples are shown in FIG. 5). The protein amounts in 10 μL of microdialysate were heterogeneous from sample to sample.

### Determination of Quantitative Cut-Offs for TMT-Based Experiments

The quantitative cut-offs that reflected significant increase and decrease in protein amount for the TMT2 experiments were evaluated experimentally (shown in FIG. 6). TMT6 were used to label two identical samples of IC, P, and CT microdialysates following the same protocol used for the TMT2 experiments that is detailed in the Experimental Procedures (i.e., reduction, alkylation, digestion, differential TMT6 labeling, off-gel electrophoresis (22, 23), RP-LC MS/MS, identification, and quantitation). After random mixing of the quantitative data, the rate of false positive at a given cut-off was assessed. For instance, 1% of false positive was found at cut-off ratios of 1.68 and 0.59 (see Experimental Procedures, and shown in FIG. 6). Finally, 0.5 and 2.0 cut-off values were chosen. These values actually corresponded to a larger interval with respect to the experimentally evaluated cut-offs, decreasing further the risk to find false positives. In addition, such differences could be assessed during validation with immunoblot.

### Qualitative and Quantitative MS Analysis

Protein samples were reduced, alkylated, digested with trypsin, and the resulting peptides were labeled with TMT2 as reported in Table 3. Off-gel electrophoresis was performed. The 12 collected off-gel fractions were analyzed with RP-LC MALDI TOF/TOF MS. The quantitative workflow was previously characterized (13, 24). The quality control of the quantitative data was evaluated with the spiked LACB protein standard (Table 3). The mean and maximum relative standard deviation of 12.1% and 17.0% (Expf) correlated with the isobaric tagging technique performances (Table 3) (13).

From these proteomic analyses, 156 proteins were identified with 939 unique peptides. More precisely, 108 proteins were identified in the IC, 137 in the P, and 134 in the CT microdialysates.

The six comparisons carried out with TMT2 showed 94 proteins, which were either increased (ratios >2.0; 53 proteins) or decreased (ratios <0.5; 47 proteins) within the compared sample pairs (Table 3). To summarize, 25 proteins were increased in IC with respect to P samples, 24 proteins were increased in IC with respect to CT samples, and 26 proteins were increased in P with respect to CT samples (Tables 7-9). The entire lists of regulated proteins between each brain region are provided in Tables 4-6.

TABLE 4
List of regulated proteins in infarct core relative to penumbra. Ratio>1 increased in IC; Ratio <1 decreased in IC

| | | | | | | nb | | |
|---|---|---|---|---|---|---|---|---|
| | | | | | | uniqe | | Corr. Ratio |
| Nr. | databank | AC | ID | MW | PI | pept | description | IC/P |
| 29 | uniprotKB_sptr | P07108 | ACBP_HUMAN | 11,793.38 | 5.71 | 5 | Acyl-CoA-binding protein (ACBP) (Diazepam-binding inhibitor) (DBI) | 1.95 |
| | | | | | | | (Endozepine) (EP). | |
| 32 | uniprotKB_sptr | P07108 | ACBP_HUMAN | 11,793.38 | 5.71 | 5 | Acyl-CoA-binding protein (ACBP) (Diazepam-binding inhibitor) (DBI) | 2.67 |
| | | | | | | | (Endozepine) (EP). | |
| 20 | uniprotKB_sptr | P02763 | A1AG1_HUMAN | 23,539.62 | 5.12 | 4 | Alpha-1-acid glycoprotein 1 precursor (AGP 1) (Orosomucoid-1) (OMD 1). | 0.48 |
| 25 | uniprotKB_sptr | P02763 | A1AG1_HUMAN | 21,560.13 | 5.10 | 3 | Alpha-1-acid glycoprotein 1 precursor (AGP 1) (Orosomucoid-1) (OMD 1). | 0.47 |
| 13 | uniprotKB_sptr | P01009_CHAIN_0 | A1AT_HUMAN | 44,324.56 | 5.44 | 8 | Alpha-1-antitrypsin precursor (Alpha-1 protease inhibitor) (Alpha-1- | 0.61 |
| | | | | | | | antiproteinase). [CHAIN 0] | |
| 30 | uniprotKB_sptr | P01009_CHAIN_0 | A1AT_HUMAN | 44,324.56 | 5.44 | 5 | Alpha-1-antitrypsin precursor (Alpha-1 protease inhibitor) (Alpha-1- | 0.40 |
| | | | | | | | antiproteinase). [CHAIN 0] | |
| 74 | uniprotKB_sptr | P04217 | A1BG_HUMAN | 51,940.72 | 5.72 | 2 | Alpha-1B-glycoprotein precursor (Alpha-1-B glycoprotein). | 0.46 |
| 71 | uniprotKB_sptr | P02765_CHAIN_0 | FETUA_HUMAN | 30,221.95 | 4.57 | 2 | Alpha-2-HS-glycoprotein precursor (Fetuin-A) (Alpha-2-Z-globulin) (Ba-alpha-2- | 1.07 |
| | | | | | | | glycoprotein) [Contains: Alpha-2-HS-glycoprotein chain A; Alpha-2-HS- | |
| | | | | | | | glycoprotein chain B]. [CHAIN 0] | |
| 8 | uniprotKB_sptr | P01023 | A2MG_HUMAN | 160,796.89 | 5.98 | 11 | Alpha-2-macroglobulin precursor (Alpha-2-M). | 0.90 |
| 6 | uniprotKB_sptr | P01023 | A2MG_HUMAN | 160,796.89 | 5.98 | 16 | Alpha-2-macroglobulin precursor (Alpha-2-M). | 0.69 |
| 47 | uniprotKB_sptr | P02647 | APOA1_HUMAN | 30,777.84 | 5.68 | 3 | Apolipoprotein A-I precursor (Apo-AI) (ApoA-I) [Contains: Apolipoprotein A-I(1- | 1.41 |
| | | | | | | | 242)]. | |
| 8 | uniprotKB_sptr | P02647_CHAIN_0 | APOA1_HUMAN | 27,950.50 | 5.36 | 12 | Apolipoprotein A-I precursor (Apo-AI) (ApoA-I) [Contains: Apolipoprotein A-I(1- | 0.79 |
| | | | | | | | 242)]. [CHAIN 0] | |
| 31 | uniprotKB_sptr | P02652 | APOA2_HUMAN | 8,579.77 | 5.39 | 4 | Apolipoprotein A-II precursor (Apo-AII) (ApoA-II) [Contains: Apolipoprotein A-II(1- | 1.03 |
| | | | | | | | 76)]. | |
| 38 | uniprotKB_sptr | P61769 | B2MG_HUMAN | 11,731.17 | 6.46 | 3 | Beta-2-microglobulin precursor [Contains: Beta-2-microglobulin form pI 5.3]. | 1.49 |
| 65 | uniprotKB_sptr | P61769 | B2MG_HUMAN | 11,731.17 | 6.46 | 2 | Beta-2-microglobulin precursor [Contains: Beta-2-microglobulin form pI 5.3]. | 2.09 |
| 66 | uniprotKB_sptr | P62158 | CALM_HUMAN | 16,706.39 | 4.12 | 2 | Calmodulin (CaM). | 1.53 |
| 59 | uniprotKB_sptr | P00915 | CAH1_HUMAN | 28,870.21 | 6.83 | 2 | Carbonic anhydrase 1 (EC 4.2.1.1) (Carbonic anhydrase I) (Carbonate | 0.45 |
| | | | | | | | dehydratase I) (CA-I). | |
| 38 | uniprotKB_sptr | P00915 | CAH1_HUMAN | 28,739.02 | 6.92 | 4 | Carbonic anhydrase 1 (EC 4.2.1.1) (Carbonic anhydrase I) (Carbonate | 1.60 |
| | | | | | | | dehydratase I) (CA-I). | |
| 42 | uniprotKB_sptr | P00918 | CAH2_HUMAN | 29,246.06 | 7.22 | 4 | Carbonic anhydrase 2 (EC 4.2.1.1) (Carbonic anhydrase II) (Carbonate | 1.89 |
| | | | | | | | dehydratase II) (CA-II) (Carbonic anhydrase C). | |
| 67 | uniprotKB_sptr | P13987 | CD59_HUMAN | 8,961.10 | 5.70 | 2 | CD59 glycoprotein precursor (Membrane attack complex inhibition factor) | 1.50 |
| | | | | | | | (MACIF) (MAC-inhibitory protein) (MAC-IP) (Protectin) (MEM43 antigen) | |
| | | | | | | | (Membrane inhibitor of reactive lysis) (MIRL) (20 kDa homologous restriction | |
| | | | | | | | factor) (HRF-20) (HRF20) (1F5 antigen). | |
| 29 | uniprotKB_sptr | P00450 | CERU_HUMAN | 122,205.23 | 5.46 | 5 | Ceruloplasmin precursor (EC 1.16.3.1) (Ferroxidase). | 0.44 |
| 11 | uniprotKB_sptr | P36222 | CH3L1_HUMAN | 40,488.87 | 8.80 | 8 | Chitinase-3-like protein 1 precursor (Cartilage glycoprotein 39) (GP- 39) (39 kDa | 0.66 |
| | | | | | | | synovial protein) (HCgp-39) (YKL-40). | |
| 54 | uniprotKB_sptr | P36222 | CH3L1_HUMAN | 40,488.87 | 8.80 | 3 | Chitinase-3-like protein 1 precursor (Cartilage glycoprotein 39) (GP- 39) (39 kDa | 1.31 |
| | | | | | | | synovial protein) (HCgp-39) (YKL-40). | |
| 70 | uniprotKB_sptr | Q14019 | COTL1_HUMAN | 15,944.98 | 5.86 | 2 | Coactosin-like protein. | 1.72 |
| 73 | uniprotKB_sptr | Q14019 | COTL1_HUMAN | 15,944.98 | 5.86 | 2 | Coactosin-like protein. | 2.04 |
| 17 | uniprotKB_sptr | P02452 | CO1A1_HUMAN | 94,766.11 | 9.36 | 7 | Collagen alpha-1(I) chain precursor (Alpha-1 type I collagen). | 1.71 |
| 37 | uniprotKB_sptr | P08123 | CO1A2_HUMAN | 91,754.77 | 10.10 | 3 | Collagen alpha-2(I) chain precursor (Alpha-2 type I collagen). | 1.97 |
| 19 | uniprotKB_sptr | P01024 | CO3_HUMAN | 187,148.13 | 6.05 | 6 | Complement C3 precursor [Contains: Complement C3 beta chain; Complement | 1.45 |
| | | | | | | | C3 alpha chain; C3a anaphylatoxin; Complement C3b alpha′ chain; Complement | |
| | | | | | | | C3c alpha′ chain fragment 1; Complement C3dg fragment; Complement C3g | |
| | | | | | | | fragment; Complement C3d fragment; Complement C3f fragment; Complement | |
| | | | | | | | C3c alpha′ chain fragment 2]. | |
| 3 | uniprotKB_sptr | P01024_CHAIN_0 | CO3_HUMAN | 184,951.41 | 6.04 | 24 | Complement C3 precursor [Contains: Complement C3 beta chain; Complement | 0.74 |
| | | | | | | | C3 alpha chain; C3a anaphylatoxin; Complement C3b alpha′ chain; Complement | |
| | | | | | | | C3c alpha′ chain fragment 1; Complement C3dg fragment; Complement C3g | |
| | | | | | | | fragment; Complement C3d fragment; Complement C3f fragment; Complement | |
| | | | | | | | C3c alpha′ chain fragment 2]. [CHAIN 0] | |
| 40 | uniprotKB_sptr | P0C0L4 | CO4A_HUMAN | 84,183.35 | 5.38 | 3 | Complement C4-A precursor (Acidic complement C4) [Contains: Complement C4 | 2.50 |
| | | | | | | | beta chain; Complement C4-A alpha chain; C4a anaphylatoxin; C4b-A; C4d-A; | |
| | | | | | | | Complement C4 gamma chain]. | |
| 24 | uniprotKB_sptr | P0C0L4 | CO4A_HUMAN | 192,742.53 | 6.72 | 6 | Complement C4-A precursor (Acidic complement C4) [Contains: Complement C4 | 1.10 |
| | | | | | | | beta chain; Complement C4-A alpha chain; C4a anaphylatoxin; C4b-A; C4d-A; | |
| | | | | | | | Complement C4 gamma chain]. | |
| 53 | uniprotKB_sptr | P08603 | CFAH_HUMAN | 137,052.63 | 6.17 | 3 | Complement factor H precursor (H factor 1). | 0.59 |
| 60 | uniprotKB_sptr | P04080 | CYTB_HUMAN | 11,139.59 | 7.91 | 2 | Cystatin-B (Stefin-B) (Liver thiol proteinase inhibitor) (CPI-B). | 2.68 |
| 22 | uniprotKB_sptr | P01034 | CYTC_HUMAN | 15,799.22 | 9.21 | 4 | Cystatin-C precursor (Cystatin-3) (Neuroendocrine basic polypeptide) (Gamma- | 1.73 |
| | | | | | | | trace) (Post-gamma-globulin). | |
| 62 | uniprotKB_sptr | P01034 | CYTC_HUMAN | 15,799.22 | 9.21 | 2 | Cystatin-C precursor (Cystatin-3) (Neuroendocrine basic polypeptide) (Gamma- | 1.86 |
| | | | | | | | trace) (Post-gamma-globulin). | |
| 48 | uniprotKB_sptr | P21291 | CSRP1_HUMAN | 20,436.21 | 8.99 | 2 | Cysteine and glycine-rich protein 1 (Cysteine-rich protein 1) (CRP1) (CRP). | 3.33 |
| 57 | uniprotKB_sptr | P21291 | CSRP1_HUMAN | 20,372.30 | 9.39 | 2 | Cysteine and glycine-rich protein 1 (Cysteine-rich protein 1) (CRP1) (CRP). | 2.88 |
| 58 | uniprotKB_sptr | O15540 | FABPB_HUMAN | 14,888.91 | 5.70 | 2 | Fatty acid-binding protein, brain (B-FABP) (Brain lipid-binding protein) (BLBP) | 2.65 |
| | | | | | | | (Mammary-derived growth inhibitor related). | |
| 12 | uniprotKB_sptr | P02671 | FIBA_HUMAN | 94,973.07 | 5.75 | 7 | Fibrinogen alpha chain precursor [Contains: Fibrinopeptide A]. | 2.97 |
| 4 | uniprotKB_sptr | P02671 | FIBA_HUMAN | 94,973.07 | 5.75 | 20 | Fibrinogen alpha chain precursor [Contains: Fibrinopeptide A]. | 0.61 |
| 13 | uniprotKB_sptr | P02675 | FIBB_HUMAN | 55,928.17 | 8.64 | 8 | Fibrinogen beta chain precursor [Contains: Fibrinopeptide B]. | 0.46 |
| 27 | uniprotKB_sptr | P02679 | FIBG_HUMAN | 49,496.55 | 5.81 | 6 | Fibrinogen gamma chain precursor. | 0.41 |
| 37 | uniprotKB_sptr | P14136 | GFAP_HUMAN | 49,880.22 | 5.47 | 3 | Glial fibrillary acidic protein (GFAP). | 1.76 |
| 63 | uniprotKB_sptr | P09211 | GSTP1_HUMAN | 23,355.84 | 5.72 | 2 | Glutathione S-transferase P (EC 2.5.1.18) (GST class-pi) (GSTP1-1). | 2.79 |
| 7 | uniprotKB_sptr | P00738 | HPT_HUMAN | 43,349.02 | 6.25 | 12 | Haptoglobin precursor [Contains: Haptoglobin alpha chain; Haptoglobin beta | 0.46 |
| | | | | | | | chain]. | |
| 5 | uniprotKB_sptr | P00738 | HPT_HUMAN | 45,205.32 | 6.24 | 16 | Haptoglobin precursor [Contains: Haptoglobin alpha chain; Haptoglobin beta | 0.36 |
| | | | | | | | chain]. | |
| 31 | uniprotKB_sptr | P69905 | HBA_HUMAN | 15,126.36 | 9.07 | 4 | Hemoglobin subunit alpha (Hemoglobin alpha chain) (Alpha-globin). | 0.34 |
| 23 | uniprotKB_sptr | P69905 | HBA_HUMAN | 15,126.36 | 9.07 | 5 | Hemoglobin subunit alpha (Hemoglobin alpha chain) (Alpha-globin). | 0.70 |
| 30 | uniprotKB_sptr | P68871 | HBB_HUMAN | 15,998.41 | 7.13 | 4 | Hemoglobin subunit beta (Hemoglobin beta chain) (Beta-globin) [Contains: LVV- | 0.37 |
| | | | | | | | hemorphin-7]. | |
| 7 | uniprotKB_sptr | P68871 | HBB_HUMAN | 15,867.22 | 7.26 | 9 | Hemoglobin subunit beta (Hemoglobin beta chain) (Beta-globin) [Contains: LVV- | 0.80 |
| | | | | | | | hemorphin-7]. | |
| 25 | uniprotKB_sptr | P02790 | HEMO_HUMAN | 51,676.39 | 6.67 | 4 | Hemopexin precursor (Beta-1B-glycoprotein). | 0.68 |
| 16 | uniprotKB_sptr | P02790 | HEMO_HUMAN | 51,676.39 | 6.67 | 7 | Hemopexin precursor (Beta-1B-glycoprotein). | 0.57 |
| 61 | uniprotKB_sptr | P38159 | HNRPG_HUMAN | 40,846.19 | 10.08 | 2 | Heterogeneous nuclear ribonucleoprotein G (hnRNP G) (RNA-binding motif | 2.35 |
| | | | | | | | protein, X chromosome) (Glycoprotein p43) [Contains: Processed heterogeneous | |
| | | | | | | | nuclear ribonucleoprotein G]. | |
| 57 | uniprotKB_sptr | Q86YZ3 | HORN_HUMAN | 282,390.06 | 10.06 | 2 | Hornerin. | 1.08 |
| 16 | uniprotKB_sptr | P01876 | IGHA1_HUMAN | 52,865.03 | 7.06 | 7 | Ig alpha-1 chain C region. | 0.76 |
| 36 | uniprotKB_sptr | P01834 | KAC_HUMAN | 25,772.87 | 6.31 | 3 | Ig kappa chain C region. | 0.51 |
| 26 | uniprotKB_sptr | P01834 | KAC_HUMAN | 13,152.70 | 7.70 | 5 | Ig kappa chain C region. | 0.28 |
| 9 | uniprotKB_sptr | Q6PI81 | Q6PI81_HUMAN | 52,666.63 | 7.87 | 4 | IGHM protein. | 0.62 |
| 75 | uniprotKB_sptr | Q14624 | ITIH4_HUMAN | 103,325.40 | 6.64 | 2 | Inter-alpha-trypsin inhibitor heavy chain H4 precursor (ITI heavy chain H4) (Inter- | 0.83 |
| | | | | | | | alpha-inhibitor heavy chain 4) (Inter-alpha-trypsin inhibitor family heavy chain- | |
| | | | | | | | related protein) (IHRP) (Plasma kallikrein sensitive glycoprotein 120) (PK-120) | |
| | | | | | | | (GP120) [Contains: 70 kDa inter-alpha-trypsin inhibitor heavy chain H4; 35 kDa | |
| | | | | | | | inter-alpha-trypsin inhibitor heavy chain H4]. | |
| 26 | uniprotKB_sptr | Q92876 | KLK6_HUMAN | 26,855.73 | 7.57 | 5 | Kallikrein-6 precursor (EC 3.4.21.—) (Protease M) (Neurosin) (Zyme) (SP59) | 0.79 |
| | | | | | | | (Serine protease 9) (Serine protease 18). | |
| 4 | uniprotKB_sptr | P13645 | K1C10_HUMAN | 56,561.89 | 5.13 | 13 | Keratin, type I cytoskeletal 10 (Cytokeratin-10) (CK-10) (Keratin-10) (K10). | 0.72 |
| 20 | uniprotKB_sptr | P13645 | K1C10_HUMAN | 56,561.89 | 5.13 | 6 | Keratin, type I cytoskeletal 10 (Cytokeratin-10) (CK-10) (Keratin-10) (K10). | 0.39 |
| 5 | uniprotKB_sptr | P35527 | K1C9_HUMAN | 62,129.47 | 5.24 | 11 | Keratin, type I cytoskeletal 9 (Cytokeratin-9) (CK-9) (Keratin-9) (K9). | 0.64 |
| 3 | uniprotKB_sptr | P04264 | K2C1_HUMAN | 66,017.70 | 8.45 | 20 | Keratin, type II cytoskeletal 1 (Cytokeratin-1) (CK-1) (Keratin-1) (K1) (67 kDa | 0.60 |
| | | | | | | | cytokeratin) (Hair alpha protein). | |
| 14 | uniprotKB_sptr | P04264 | K2C1_HUMAN | 66,017.70 | 8.45 | 5 | Keratin, type II cytoskeletal 1 (Cytokeratin-1) (CK-1) (Keratin-1) (K1) (67 kDa | 0.51 |
| | | | | | | | cytokeratin) (Hair alpha protein). | |
| 6 | uniprotKB_sptr | P35908 | K22E_HUMAN | 65,865.35 | 8.35 | 9 | Keratin, type II cytoskeletal 2 epidermal (Cytokeratin-2e) (K2e) (CK 2e) (keratin- | 0.68 |
| | | | | | | | 2). | |
| 21 | uniprotKB_sptr | A2NUT2 | A2NUT2_HUMAN | 24,960.80 | 5.40 | 5 | Lambda-chain precursor (AA −20 to 215). | 0.74 |
| 28 | uniprotKB_sptr | A2NUT2 | A2NUT2_HUMAN | 25,020.97 | 8.45 | 2 | Lambda-chain precursor (AA −20 to 215). | 0.66 |
| 41 | uniprotKB_sptr | P61626 | LYSC_HUMAN | 16,537.02 | 9.50 | 4 | Lysozyme C precursor (EC 3.2.1.17) (1,4-beta-N-acetylmuramidase C). | 1.42 |
| 35 | uniprotKB_sptr | P25713 | MT3_HUMAN | 6,926.97 | 5.00 | 3 | Metallothionein-3 (MT-3) (Metallothionein-III) (MT-III) (Growth inhibitory factor) | 2.10 |
| | | | | | | | (GIF) (GIFB). | |
| 44 | uniprotKB_sptr | P25713 | MT3_HUMAN | 6,926.97 | 5.00 | 3 | Metallothionein-3 (MT-3) (Metallothionein-III) (MT-III) (Growth inhibitory factor) | 2.79 |
| | | | | | | | (GIF) (GIFB). | |
| 51 | uniprotKB_sptr | P02686_ISOFORM_3 | MBP_HUMAN | 21,493.21 | 11.45 | 2 | Myelin basic protein (MBP) (Myelin A1 protein) (Myelin membrane | 1.71 |
| | | | | | | | encephalitogenic protein). [ISOFORM 3] | |
| 36 | uniprotKB_sptr | P02686_ISOFORM_3 | MBP_HUMAN | 20,245.79 | 11.27 | 2 | Myelin basic protein (MBP) (Myelin A1 protein) (Myelin membrane | 3.11 |
| | | | | | | | encephalitogenic protein). [ISOFORM 3] | |
| 43 | uniprotKB_sptr | O94760 | DDAH1_HUMAN | 31,121.78 | 5.64 | 3 | N(G),N(G)-dimethylarginine dimethylaminohydrolase 1 (EC 3.5.3.18) | 1.26 |
| | | | | | | | (Dimethylargininase-1) (Dimethylarginine dimethylaminohydrolase 1) (DDAHI) | |
| | | | | | | | (DDAH-1). | |
| 62 | uniprotKB_sptr | P07197 | NFM_HUMAN | 102,316.84 | 4.90 | 2 | Neurofilament medium polypeptide (NF-M) (Neurofilament triplet M protein) (160 kDa | 1.54 |
| | | | | | | | neurofilament protein) (Neurofilament 3). | |
| 66 | uniprotKB_sptr | P59665 | DEF1_HUMAN | 6,350.36 | 8.32 | 2 | Neutrophil defensin 1 precursor (HNP-1) (HP-1) (HP1) (Defensin, alpha 1) | 2.45 |
| | | | | | | | [Contains: HP 1-56; Neutrophil defensin 2 (HNP-2) (HP-2) (HP2)]. | |
| 24 | uniprotKB_sptr | P10451 | OSTP_HUMAN | 33,016.56 | 4.59 | 4 | Osteopontin precursor (Bone sialoprotein 1) (Secreted phosphoprotein 1) (SPP- | 1.50 |
| | | | | | | | 1) (Urinary stone protein) (Nephropontin) (Uropontin). | |
| 54 | uniprotKB_sptr | O75781 | PALM_HUMAN | 37,157.28 | 4.96 | 2 | Paralemmin. | 3.52 |
| 15 | uniprotKB_sptr | P62937 | PPIA_HUMAN | 18,012.50 | 8.34 | 7 | Peptidyl-prolyl cis-trans isomerase A (EC 5.2.1.8) (PPlase A) (Rotamase A) | 2.45 |
| | | | | | | | (Cyclophilin A) (Cyclosporin A-binding protein). | |
| 34 | uniprotKB_sptr | P62937 | PPIA_HUMAN | 18,012.50 | 8.34 | 5 | Peptidyl-prolyl cis-trans isomerase A (EC 5.2.1.8) (PPlase A) (Rotamase A) | 1.60 |
| | | | | | | | (Cyclophilin A) (Cyclosporin A-binding protein). | |
| 72 | uniprotKB_sptr | Q06830 | PRDX1_HUMAN | 22,110.36 | 8.59 | 2 | Peroxiredoxin-1 (EC 1.11.1.15) (Thioredoxin peroxidase 2) (Thioredoxin- | 1.93 |
| | | | | | | | dependent peroxide reductase 2) (Proliferation-associated gene protein) (PAG) | |
| | | | | | | | (Natural killer cell-enhancing factor A) (NKEF-A). | |
| 60 | uniprotKB_sptr | P32119 | PRDX2_HUMAN | 16,102.26 | 6.74 | 2 | Peroxiredoxin-2 (EC 1.11.1.15) (Thioredoxin peroxidase 1) (Thioredoxin- | 2.72 |
| | | | | | | | dependent peroxide reductase 1) (Thiol-specific antioxidant protein) (TSA) (PRP) | |
| | | | | | | | (Natural killer cell-enhancing factor B) (NKEF-B). | |
| 45 | uniprotKB_sptr | P30041 | PRDX6_HUMAN | 25,034.99 | 6.34 | 3 | Peroxiredoxin-6 (EC 1.11.1.15) (Antioxidant protein 2) (1-Cys peroxiredoxin) (1- | 2.15 |
| | | | | | | | Cys PRX) (Acidic calcium-independent phospholipase A2) (EC 3.1.1.—) (aiPLA2) | |
| | | | | | | | (Non-selenium glutathione peroxidase) (EC 1.11.1.7) (NSGPx) (24 kDa protein) | |
| | | | | | | | (Liver 2D page spot 40) (Red blood cells page spot 12). | |
| 56 | uniprotKB_sptr | P30041 | PRDX6_HUMAN | 25,034.99 | 6.34 | 3 | Peroxiredoxin-6 (EC 1.11.1.15) (Antioxidant protein 2) (1-Cys peroxiredoxin) (1- | 2.16 |
| | | | | | | | Cys PRX) (Acidic calcium-independent phospholipase A2) (EC 3.1.1.—) (aiPLA2) | |
| | | | | | | | (Non-selenium glutathione peroxidase) (EC 1.11.1.7) (NSGPx) (24 kDa protein) | |
| | | | | | | | (Liver 2D page spot 40) (Red blood cells page spot 12). | |
| 10 | uniprotKB_sptr | P30086 | PEBP1_HUMAN | 21,056.79 | 7.76 | 7 | Phosphatidylethanolamine-binding protein 1 (PEBP-1) (Prostatic-binding protein) | 2.06 |
| | | | | | | | (HCNPpp) (Neuropolypeptide h3) (Raf kinase inhibitor protein) (RKIP) [Contains: | |
| | | | | | | | Hippocampal cholinergic neurostimulating peptide (HCNP)]. | |
| 21 | uniprotKB_sptr | P30086 | PEBP1_HUMAN | 21,056.79 | 7.76 | 7 | Phosphatidylethanolamine-binding protein 1 (PEBP-1) (Prostatic-binding protein) | 1.60 |
| | | | | | | | (HCNPpp) (Neuropolypeptide h3) (Raf kinase inhibitor protein) (RKIP) [Contains: | |
| | | | | | | | Hippocampal cholinergic neurostimulating peptide (HCNP)]. | |
| 59 | uniprotKB_sptr | P05155 | IC1_HUMAN | 52,843.38 | 6.10 | 2 | Plasma protease C1 inhibitor precursor (C1 Inh) (C1Inh) (C1 esterase inhibitor) | 0.79 |
| | | | | | | | (C1-inhibiting factor). | |
| 72 | uniprotKB_sptr | P02753 | RETBP_HUMAN | 21,071.60 | 5.48 | 2 | Plasma retinol-binding protein precursor (PRBP) (RBP) [Contains: Plasma retinol- | 2.83 |
| | | | | | | | binding protein(1-182); Plasma retinol-binding protein(1-181); Plasma retinol- | |
| | | | | | | | binding protein(1-179); Plasma retinol-binding protein(1-176)]. | |
| 77 | uniprotKB_sptr | P02753 | RETBP_HUMAN | 22,933.85 | 6.73 | 2 | Plasma retinol-binding protein precursor (PRBP) (RBP) [Contains: Plasma retinol- | 1.63 |
| | | | | | | | binding protein(1-182); Plasma retinol-binding protein(1-181); Plasma retinol- | |
| | | | | | | | binding protein(1-179); Plasma retinol-binding protein(1-176)]. | |
| 55 | uniprotKB_sptr | P00747 | PLMN_HUMAN | 15,443.72 | 8.91 | 2 | Plasminogen precursor (EC 3.4.21.7) [Contains: Plasmin heavy chain A; | 2.27 |
| | | | | | | | Activation peptide; Angiostatin; Plasmin heavy chain A, short form; Plasmin light | |
| | | | | | | | chain B]. | |
| 49 | uniprotKB_sptr | P02775 | SCYB7_HUMAN | 9,105.57 | 8.74 | 2 | Platelet basic protein precursor (PBP) (C—X—C motif chemokine 7) (Small- | 2.51 |
| | | | | | | | inducible cytokine B7) (Leukocyte-derived growth factor) (LDGF) (Macrophage- | |
| | | | | | | | derived growth factor) (MDGF) [Contains: Connective tissue-activating peptide III | |
| | | | | | | | (CTAP-III) (Low-affinity platelet factor IV) (LA-PF4); TC-2; Connective tissue- | |
| | | | | | | | activating peptide III(1-81) (CTAP-III(1-81)); Beta-thromboglobulin (Beta-TG); | |
| | | | | | | | Neutrophil-activating peptide 2(74) (NAP-2(74)); Neutrophil-activating peptide | |
| | | | | | | | 2(73) (NAP-2(73)); Neutrophil-activating peptide 2 (NAP-2); TC-1; Neutrophil- | |
| | | | | | | | activating peptide 2(1-66) (NAP-2(1-66)); Neutrophil-activating peptide 2(1-63) | |
| | | | | | | | (NAP-2(1-63))]. | |
| 49 | uniprotKB_sptr | P02775 | SCYB7_HUMAN | 9,105.57 | 8.74 | 3 | Platelet basic protein precursor (PBP) (C—X—C motif chemokine 7) (Small- | 0.85 |
| | | | | | | | inducible cytokine B7) (Leukocyte-derived growth factor) (LDGF) (Macrophage- | |
| | | | | | | | derived growth factor) (MDGF) [Contains: Connective tissue-activating peptide III | |
| | | | | | | | (CTAP-III) (Low-affinity platelet factor IV) (LA-PF4); TC-2; Connective tissue- | |
| | | | | | | | activating peptide III(1-81) (CTAP-III(1-81)); Beta-thromboglobulin (Beta-TG); | |
| | | | | | | | Neutrophil-activating peptide 2(74) (NAP-2(74)); Neutrophil-activating peptide | |
| | | | | | | | 2(73) (NAP-2(73)); Neutrophil-activating peptide 2 (NAP-2); TC-1; Neutrophil- | |
| | | | | | | | activating peptide 2(1-66) (NAP-2(1-66)); Neutrophil-activating peptide 2(1-63) | |
| | | | | | | | (NAP-2(1-63))]. | |
| 34 | uniprotKB_sptr | P07602 | SAP_HUMAN | 26,308.66 | 5.88 | 3 | Proactivator polypeptide precursor [Contains: Saposin-A (Protein A); Saposin-B- | 1.60 |
| | | | | | | | Val; Saposin-B (Sphingolipid activator protein 1) (SAP-1) (Cerebroside sulfate | |
| | | | | | | | activator) (CSAct) (Dispersin) (Sulfatide/GM1 activator); Saposin-C (Co-beta- | |
| | | | | | | | glucosidase) (A1 activator) (Glucosylceramidase activator) (Sphingolipid activator | |
| | | | | | | | protein 2) (SAP-2); Saposin-D (Protein C) (Component C)]. | |
| 58 | uniprotKB_sptr | P07602 | SAP_HUMAN | 26,308.66 | 5.88 | 3 | Proactivator polypeptide precursor [Contains: Saposin-A (Protein A); Saposin-B- | 1.25 |
| | | | | | | | Val; Saposin-B (Sphingolipid activator protein 1) (SAP-1) (Cerebroside sulfate | |
| | | | | | | | activator) (CSAct) (Dispersin) (Sulfatide/GM1 activator); Saposin-C (Co-beta- | |
| | | | | | | | glucosidase) (A1 activator) (Glucosylceramidase activator) (Sphingolipid activator | |
| | | | | | | | protein 2) (SAP-2); Saposin-D (Protein C) (Component C)]. | |
| 53 | uniprotKB_sptr | P07737 | PROF1_HUMAN | 15,054.23 | 8.79 | 2 | Profilin-1 (Profilin I). | 2.40 |
| 46 | uniprotKB_sptr | P07737 | PROF1_HUMAN | 15,054.23 | 8.79 | 3 | Profilin-1 (Profilin I). | 0.91 |
| 28 | uniprotKB_sptr | P41222_CHAIN_0 | PTGDS_HUMAN | 18,698.03 | 8.68 | 4 | Prostaglandin-H2 D-isomerase precursor (EC 5.3.99.2) (Lipocalin-type | 0.84 |
| | | | | | | | prostaglandin-D synthase) (Glutathione-independent PGD synthetase) | |
| | | | | | | | (Prostaglandin-D2 synthase) (PGD2 synthase) (PGDS2) (PGDS) (Beta-trace | |
| | | | | | | | protein) (Cerebrin-28). [CHAIN 0] | |
| 50 | uniprotKB_sptr | P41222_CHAIN_0 | PTGDS_HUMAN | 18,698.03 | 8.68 | 3 | Prostaglandin-H2 D-isomerase precursor (EC 5.3.99.2) (Lipocalin-type | 1.63 |
| | | | | | | | prostaglandin-D synthase) (Glutathione-independent PGD synthetase) | |
| | | | | | | | (Prostaglandin-D2 synthase) (PGD2 synthase) (PGDS2) (PGDS) (Beta-trace | |
| | | | | | | | protein) (Cerebrin-28). [CHAIN 0] | |
| 64 | uniprotKB_sptr | A0N5G5 | A0N5G5_HUMAN | 12,766.37 | 9.45 | 2 | Rheumatoid factor D5 light chain (Fragment). | 0.96 |
| 67 | uniprotKB_sptr | A0N5G5 | A0N5G5_HUMAN | 12,766.37 | 9.45 | 2 | Rheumatoid factor D5 light chain (Fragment). | 0.75 |
| 2 | uniprotKB_sptr | P02787 | TRFE_HUMAN | 76,959.79 | 7.16 | 29 | Serotransferrin precursor (Transferrin) (Siderophilin) (Beta-1-metal-binding | 0.42 |
| | | | | | | | globulin). | |
| 2 | uniprotKB_sptr | P02787 | TRFE_HUMAN | 77,049.89 | 6.97 | 24 | Serotransferrin precursor (Transferrin) (Siderophilin) (Beta-1-metal-binding | 0.50 |
| | | | | | | | globulin). | |
| 1 | uniprotKB_sptr | P02768 | ALBU_HUMAN | 66,472.22 | 5.73 | 46 | Serum albumin precursor. | 0.40 |
| 1 | uniprotKB_sptr | P02768 | ALBU_HUMAN | 69,366.70 | 5.98 | 49 | Serum albumin precursor. | 0.33 |
| 33 | uniprotKB_sptr | O75368 | SH3L1_HUMAN | 12,774.25 | 5.53 | 3 | SH3 domain-binding glutamic acid-rich-like protein. | 2.17 |
| 48 | uniprotKB_sptr | O75368 | SH3L1_HUMAN | 12,774.25 | 5.53 | 3 | SH3 domain-binding glutamic acid-rich-like protein. | 1.92 |
| 32 | uniprotKB_sptr | P00441 | SODC_HUMAN | 16,122.00 | 6.04 | 4 | Superoxide dismutase [Cu—Zn] (EC 1.15.1.1). | 1.18 |
| 71 | uniprotKB_sptr | P10599 | THIO_HUMAN | 9,451.99 | 6.56 | 2 | Thioredoxin (Trx) (ATL-derived factor) (ADF) (Surface-associated sulphydryl | 2.17 |
| | | | | | | | protein) (SASP). | |
| 46 | uniprotKB_sptr | P04216 | THY1_HUMAN | 12,553.22 | 9.33 | 2 | Thy-1 membrane glycoprotein precursor (Thy-1 antigen) (CD90 antigen) | 1.29 |
| | | | | | | | (CDw90). | |
| 68 | uniprotKB_sptr | P62328 | TYB4_HUMAN | 7,312.18 | 8.84 | 2 | Thymosin beta-4 (T beta 4) (Fx) [Contains: Hematopoietic system regulatory | 1.69 |
| | | | | | | | peptide (Seraspenide)]. | |
| 41 | uniprotKB_sptr | P60174 | TPIS_HUMAN | 26,669.50 | 6.90 | 3 | Triosephosphate isomerase (EC 5.3.1.1) (TIM) (Triose-phosphate isomerase). | 0.82 |
| 51 | uniprotKB_sptr | P60174 | TPIS_HUMAN | 27,126.45 | 8.70 | 3 | Triosephosphate isomerase (EC 5.3.1.1) (TIM) (Triose-phosphate isomerase). | 0.68 |
| 27 | uniprotKB_sptr | P62988 | UBIQ_HUMAN | 61,523.80 | 7.25 | 4 | Ubiquitin. | 2.09 |
| 39 | uniprotKB_sptr | P62988 | UBIQ_HUMAN | 25,761.65 | 7.78 | 4 | Ubiquitin. | 1.50 |
| 15 | uniprotKB_sptr | A6NGU3 | A6NGU3_HUMAN | 57,019.61 | 8.48 | 2 | Uncharacterized protein IGHG3 (Fragment). | 0.61 |
| 65 | uniprotKB_sptr | Q9GZP4 | CA128_HUMAN | 24,177.85 | 5.58 | 2 | UPF0424 protein C1orf128. | 1.76 |
| 43 | uniprotKB_sptr | P02774 | VTDB_HUMAN | 52,963.66 | 5.47 | 4 | Vitamin D-binding protein precursor (DBP) (Group-specific component) (Gc- | 0.63 |
| | | | | | | | globulin) (VDB). | |
| 69 | uniprotKB_sptr | P04004_CHAIN_0 | VTNC_HUMAN | 43,029.62 | 5.64 | 2 | Vitronectin precursor (Serum-spreading factor) (S-protein) (V75) [Contains: | 1.47 |
| | | | | | | | Vitronectin V65 subunit; Vitronectin V10 subunit; Somatomedin-B]. [CHAIN 0] | |
| 76 | uniprotKB_sptr | P25311 | ZA2G_HUMAN | 34,244.70 | 5.85 | 2 | Zinc-alpha-2-glycoprotein precursor (Zn-alpha-2-glycoprotein) (Zn-alpha-2-GP). | 0.56 |

TABLE 5
List of regulated proteins in infarct core relative to contralateral hemisphere. Ratio >1 increased in IC; Ratio <1 decreased in IC

| | | | | | | nb | | |
|---|---|---|---|---|---|---|---|---|
| | | | | | | uniqe | | Corr. Ratio |
| Nr. | databank | AC | ID | MW | PI | pept | description | IC/P |
| 20 | uniprotKB_sptr | P60709 | ACTB_HUMAN | 41,004.97 | 5.67 | 4 | Actin, cytoplasmic 1 (Beta-actin). | 1.65 |
| 26 | uniprotKB_sptr | P07108 | ACBP_HUMAN | 11793.375 | 5.71 | 3 | Acyl-CoA-binding protein (ACBP) (Diazepam-binding inhibitor) (DBI) (Endozepine) (EP). | 12.53 |
| 26 | uniprotKB_sptr | P01009 | A1AT_HUMAN | 22,828.35 | 6.35 | 4 | Alpha-1-antitrypsin precursor (Alpha-1 protease inhibitor) (Alpha-1- | 0.49 |
| | | | | | | | antiproteinase). | |
| 50 | uniprotKB_sptr | P01023 | A2MG_HUMAN | 160,796.89 | 5.98 | 2 | Alpha-2-macroglobulin precursor (Alpha-2-M). | 0.36 |
| 40 | uniprotKB_sptr | P02760 | AMBP_HUMAN | 38,999.49 | 6.18 | 3 | AMBP protein precursor [Contains: Alpha-1-microglobulin (Protein HC) (Complex- | 1.82 |
| | | | | | | | forming glycoprotein heterogeneous in charge) (Alpha-1 microglycoprotein); Inter- | |
| | | | | | | | alpha-trypsin inhibitor light chain (ITI-LC) (Bikunin) (HI-30)]. | |
| 27 | uniprotKB_sptr | Q5EFE5 | Q5EFE5_HUMAN | 52362.454 | 8.73 | 2 | Anti-RhD monoclonal T125 gamma1 heavy chain precursor. | 0.82 |
| 24 | uniprotKB_sptr | P02647 | APOA1_HUMAN | 27,950.50 | 5.36 | 4 | Apolipoprotein A-I precursor (Apo-AI) (ApoA-I) [Contains: Apolipoprotein A-I(1-242)]. | 0.80 |
| 59 | uniprotKB_sptr | P02652 | APOA2_HUMAN | 8,579.77 | 5.39 | 2 | Apolipoprotein A-II precursor (Apo-AII) (ApoA-II) [Contains: Apolipoprotein A-II(1-76)]. | 2.28 |
| 60 | uniprotKB_sptr | P06727 | APOA4_HUMAN | 28,157.53 | 5.45 | 2 | Apolipoprotein A-IV precursor (Apo-AIV) (ApoA-IV). | 3.79 |
| 33 | uniprotKB_sptr | P61769 | B2MG_HUMAN | 11731.167 | 6.46 | 2 | Beta-2-microglobulin precursor [Contains: Beta-2-microglobulin form pI 5.3]. | 0.80 |
| 53 | uniprotKB_sptr | P61769 | B2MG_HUMAN | 11,731.17 | 6.46 | 2 | Beta-2-microglobulin precursor [Contains: Beta-2-microglobulin form pI 5.3]. | 0.44 |
| 25 | uniprotKB_sptr | P62158 | CALM_HUMAN | 16706.394 | 4.12 | 3 | Calmodulin (CaM). | 1.06 |
| 19 | uniprotKB_sptr | P00915 | CAH1_HUMAN | 28739.022 | 6.92 | 4 | Carbonic anhydrase 1 (EC 4.2.1.1) (Carbonic anhydrase I) (Carbonate | 0.29 |
| | | | | | | | dehydratase I) (CA-I). | |
| 12 | uniprotKB_sptr | P00915 | CAH1_HUMAN | 28,739.02 | 6.92 | 5 | Carbonic anhydrase 1 (EC 4.2.1.1) (Carbonic anhydrase I) (Carbonate | 4.68 |
| | | | | | | | dehydratase I) (CA-I). | |
| 28 | uniprotKB_sptr | P00918 | CAH2_HUMAN | 29,246.06 | 7.22 | 4 | Carbonic anhydrase 2 (EC 4.2.1.1) (Carbonic anhydrase II) (Carbonate | 3.18 |
| | | | | | | | dehydratase II) (CA-II) (Carbonic anhydrase C). | |
| 56 | uniprotKB_sptr | P49913 | CAMP_HUMAN | 19,301.39 | 9.59 | 2 | Cathelicidin antimicrobial peptide precursor (18 kDa cationic antimicrobial protein) | 1.53 |
| | | | | | | | (CAP-18) (hCAP-18) [Contains: Antibacterial protein FALL-39 (FALL-39 peptide | |
| | | | | | | | antibiotic); Antibacterial protein LL-37]. | |
| 9 | uniprotKB_sptr | P36222 | CH3L1_HUMAN | 40488.867 | 8.80 | 6 | Chitinase-3-like protein 1 precursor (Cartilage glycoprotein 39) (GP-39) (39 kDa | 2.44 |
| | | | | | | | synovial protein) (HCgp-39) (YKL-40). | |
| 39 | uniprotKB_sptr | P10645 | CMGA_HUMAN | 48960.312 | 4.57 | 2 | Chromogranin-A precursor (CgA) (Pituitary secretory protein I) (SP-I) [Contains: | 0.29 |
| | | | | | | | Vasostatin-1 (Vasostatin I); Vasostatin-2 (Vasostatin II); EA-92; ES-43; | |
| | | | | | | | Pancreastatin; SS-18; WA-8; WE-14; LF-19; AL-11; GV-19; GR-44; ER-37]. | |
| 63 | uniprotKB_sptr | P23528 | COF1_HUMAN | 18,502.50 | 8.53 | 2 | Cofilin-1 (Cofilin, non-muscle isoform) (18 kDa phosphoprotein) (p18). | 2.00 |
| 16 | uniprotKB_sptr | P01024 | CO3_HUMAN | 184,951.41 | 6.04 | 5 | Complement C3 precursor [Contains: Complement C3 beta chain; Complement | 0.43 |
| | | | | | | | C3 alpha chain; C3a anaphylatoxin; Complement C3b alpha′ chain; Complement | |
| | | | | | | | C3c alpha′ chain fragment 1; Complement C3dg fragment; Complement C3g | |
| | | | | | | | fragment; Complement C3d fragment; Complement C3f fragment; Complement | |
| | | | | | | | C3c alpha′ chain fragment 2]. | |
| 49 | uniprotKB_sptr | P04080 | CYTB_HUMAN | 11,139.59 | 7.91 | 2 | Cystatin-B (Stefin-B) (Liver thiol proteinase inhibitor) (CPI-B). | 2.10 |
| 24 | uniprotKB_sptr | P01034_CHAIN_0 | CYTC_HUMAN | 13347.14 | 9.00 | 3 | Cystatin-C precursor (Cystatin-3) (Neuroendocrine basic polypeptide) (Gamma- | 0.62 |
| | | | | | | | trace) (Post-gamma-globulin). [CHAIN 0] | |
| 31 | uniprotKB_sptr | P01034 | CYTC_HUMAN | 15,799.22 | 9.21 | 3 | Cystatin-C precursor (Cystatin-3) (Neuroendocrine basic polypeptide) (Gamma- | 0.03 |
| | | | | | | | trace) (Post-gamma-globulin). | |
| 20 | uniprotKB_sptr | P81605 | DCD_HUMAN | 11283.862 | 6.72 | 4 | Dermcidin precursor (Preproteolysin) [Contains: Survival-promoting peptide; | 0.85 |
| | | | | | | | DCD-1]. | |
| 61 | uniprotKB_sptr | P81605 | DCD_HUMAN | 4,705.34 | 5.61 | 2 | Dermcidin precursor (Preproteolysin) [Contains: Survival-promoting peptide; | 0.35 |
| | | | | | | | DCD-1]. | |
| 14 | uniprotKB_sptr | P02671 | FIBA_HUMAN | 94973.068 | 5.75 | 5 | Fibrinogen alpha chain precursor [Contains: Fibrinopeptide A] | 0.46 |
| 11 | uniprotKB_sptr | P02671 | FIBA_HUMAN | 94,973.07 | 5.75 | 5 | Fibrinogen alpha chain precursor [Contains: Fibrinopeptide A]. | 2.33 |
| 47 | uniprotKB_sptr | P02675 | FIBB_HUMAN | 55,928.17 | 8.64 | 2 | Fibrinogen beta chain precursor [Contains: Fibrinopeptide B]. | 0.38 |
| 48 | uniprotKB_sptr | P30043 | BLVRB_HUMAN | 22,119.35 | 7.86 | 2 | Flavin reductase (EC 1.5.1.30) (FR) (NADPH-dependent diaphorase) (NADPH- | 2.22 |
| | | | | | | | flavin reductase) (FLR) (Biliverdin reductase B) (EC 1.3.1.24) (BVR-B) (Biliverdin- | |
| | | | | | | | IX beta-reductase) (Green heme-binding protein) (GHBP). | |
| 15 | uniprotKB_sptr | P14136 | GFAP_HUMAN | 49880.22 | 5.47 | 4 | Glial fibrillary acidic protein (GFAP). | 4.13 |
| 38 | uniprotKB_sptr | P14136 | GFAP_HUMAN | 49,505.89 | 5.95 | 2 | Glial fibrillary acidic protein (GFAP). | 0.37 |
| 13 | uniprotKB_sptr | P00738 | HPT_HUMAN | 45205.318 | 6.24 | 5 | Haptoglobin precursor [Contains: Haptoglobin alpha chain; Haptoglobin beta | 0.83 |
| | | | | | | | chain]. | |
| 41 | uniprotKB_sptr | P00738 | HPT_HUMAN | 45,205.32 | 6.24 | 3 | Haptoglobin precursor [Contains: Haptoglobin alpha chain; Haptoglobin beta | 0.14 |
| | | | | | | | chain]. | |
| 42 | uniprotKB_sptr | P69905 | HBA_HUMAN | 15,126.36 | 9.07 | 2 | Hemoglobin subunit alpha (Hemoglobin alpha chain) (Alpha-globin). | 3.21 |
| 40 | uniprotKB_sptr | P68871 | HBB_HUMAN | 15867.217 | 7.26 | 2 | Hemoglobin subunit beta (Hemoglobin beta chain) (Beta-globin) [Contains: LVV- | 0.81 |
| | | | | | | | hemorphin-7]. | |
| 13 | uniprotKB_sptr | P16403 | H12_HUMAN | 21,233.57 | 11.02 | 2 | Histone H1.2 (Histone H1d). | 2.97 |
| 14 | uniprotKB_sptr | P16401 | H15_HUMAN | 22,448.99 | 10.98 | 4 | Histone H1.5 (Histone H1a). | 2.03 |
| 15 | uniprotKB_sptr | P01859 | IGHG2_HUMAN | 35,884.65 | 8.01 | 2 | Ig gamma-2 chain C region. | 0.18 |
| 16 | uniprotKB_sptr | Q92876 | KLK6_HUMAN | 24499.85 | 7.26 | 5 | Kallikrein-6 precursor (EC 3.4.21.—) (Protease M) (Neurosin) (Zyme) (SP59) | 0.60 |
| | | | | | | | (Serine protease 9) (Serine protease 18). | |
| 4 | uniprotKB_sptr | P13645 | K1C10_HUMAN | 59510.708 | 5.18 | 16 | Keratin, type I cytoskeletal 10 (Cytokeratin-10) (CK-10) (Keratin-10) (K10). | 0.99 |
| 5 | uniprotKB_sptr | P13645 | K1C10_HUMAN | 59,510.71 | 5.18 | 9 | Keratin, type I cytoskeletal 10 (Cytokeratin-10) (CK-10) (Keratin-10) (K10). | 0.48 |
| 7 | uniprotKB_sptr | P02533 | K1C14_HUMAN | 51490.331 | 5.13 | 6 | Keratin, type I cytoskeletal 14 (Cytokeratin-14) (CK-14) (Keratin-14) (K14). | 0.91 |
| 10 | uniprotKB_sptr | P08779 | K1C16_HUMAN | 51,267.84 | 5.03 | 6 | Keratin, type I cytoskeletal 16 (Cytokeratin-16) (CK-16) (Keratin-16) (K16). | 1.28 |
| 5 | uniprotKB_sptr | P35527 | K1C9_HUMAN | 62129.473 | 5.24 | 14 | Keratin, type I cytoskeletal 9 (Cytokeratin-9) (CK-9) (Keratin-9) (K9). | 0.84 |
| 4 | uniprotKB_sptr | P35527 | K1C9_HUMAN | 62,129.47 | 5.24 | 10 | Keratin, type I cytoskeletal 9 (Cytokeratin-9) (CK-9) (Keratin-9) (K9). | 0.67 |
| 2 | uniprotKB_sptr | P04264 | K2C1_HUMAN | 66017.701 | 8.45 | 23 | Keratin, type II cytoskeletal 1 (Cytokeratin-1) (CK-1) (Keratin-1) (K1) (67 kDa | 0.96 |
| | | | | | | | cytokeratin) (Hair alpha protein). | |
| 2 | uniprotKB_sptr | P04264 | K2C1_HUMAN | 66,017.70 | 8.45 | 16 | Keratin, type II cytoskeletal 1 (Cytokeratin-1) (CK-1) (Keratin-1) (K1) (67 kDa | 0.52 |
| | | | | | | | cytokeratin) (Hair alpha protein). | |
| 3 | uniprotKB_sptr | P35908 | K22E_HUMAN | 65865.354 | 8.35 | 13 | Keratin, type II cytoskeletal 2 epidermal (Cytokeratin-2e) (K2e) (CK 2e) (keratin- | 0.97 |
| | | | | | | | 2). | |
| 3 | uniprotKB_sptr | P35908 | K22E_HUMAN | 65,865.35 | 8.35 | 7 | Keratin, type II cytoskeletal 2 epidermal (Cytokeratin-2e) (K2e) (CK 2e) (keratin- | 0.43 |
| | | | | | | | 2). | |
| 8 | uniprotKB_sptr | P13647 | K2C5_HUMAN | 62378.367 | 8.14 | 2 | Keratin, type II cytoskeletal 5 (Cytokeratin-5) (CK-5) (Keratin-5) (K5) (58 kDa | 0.75 |
| | | | | | | | cytokeratin). | |
| 6 | uniprotKB_sptr | P13647 | K2C5_HUMAN | 62,378.37 | 8.14 | 4 | Keratin, type II cytoskeletal 5 (Cytokeratin-5) (CK-5) (Keratin-5) (K5) (58 kDa | 0.63 |
| | | | | | | | cytokeratin). | |
| 8 | uniprotKB_sptr | P02538 | K2C6A_HUMAN | 60,044.97 | 8.38 | 2 | Keratin, type II cytoskeletal 6A (Cytokeratin-6A) (CK 6A) (K6a keratin) | 1.07 |
| | | | | | | | (Cytokeratin-6D) (CK 6D). | |
| 22 | uniprotKB_sptr | A2NUT2 | A2NUT2_HUMAN | 25,020.97 | 8.45 | 2 | Lambda-chain precursor (AA −20 to 215). | 0.21 |
| 9 | uniprotKB_sptr | P61626 | LYSC_HUMAN | 16,537.02 | 9.50 | 4 | Lysozyme C precursor (EC 3.2.1.17) (1,4-beta-N-acetylmuramidase C). | 2.75 |
| 36 | uniprotKB_sptr | P25713 | MT3_HUMAN | 6926.967 | 5.00 | 2 | Metallothionein-3 (MT-3) (Metallothionein-III) (MT-III) (Growth inhibitory factor) | 1.39 |
| | | | | | | | (GIF) (GIFB). | |
| 54 | uniprotKB_sptr | P02686 | MBP_HUMAN | 17,343.36 | 11.29 | 2 | Myelin basic protein (MBP) (Myelin A1 protein) (Myelin membrane | 0.30 |
| | | | | | | | encephalitogenic protein). | |
| 41 | uniprotKB_sptr | O94760 | DDAH1_HUMAN | 31121.782 | 5.64 | 2 | N(G),N(G)-dimethylarginine dimethylaminohydrolase 1 (EC 3.5.3.18) | 6.31 |
| | | | | | | | (Dimethylargininase-1) (Dimethylarginine dimethylaminohydrolase 1) (DDAHI) | |
| | | | | | | | (DDAH-1). | |
| 11 | uniprotKB_sptr | P07197 | NFM_HUMAN | 102316.835 | 4.90 | 7 | Neurofilament medium polypeptide (NF-M) (Neurofilament triplet M protein) (160 kDa | 4.68 |
| | | | | | | | neurofilament protein) (Neurofilament 3). | |
| 44 | uniprotKB_sptr | P59665 | DEF1_HUMAN | 6,350.36 | 8.32 | 2 | Neutrophil defensin 1 precursor (HNP-1) (HP-1) (HP1) (Defensin, alpha 1) | 4.23 |
| | | | | | | | [Contains: HP 1-56; Neutrophil defensin 2 (HNP-2) (HP-2) (HP2)]. | |
| 43 | uniprotKB_sptr | P80188 | NGAL_HUMAN | 22,902.42 | 9.15 | 2 | Neutrophil gelatinase-associated lipocalin precursor (NGAL) (p25) (25 kDa alpha- | 1.85 |
| | | | | | | | 2-microglobulin-related subunit of MMP-9) (Lipocalin-2) (Oncogene 24p3). | |
| 18 | uniprotKB_sptr | P10451 | OSTP_HUMAN | 33016.56 | 4.59 | 3 | Osteopontin precursor (Bone sialoprotein 1) (Secreted phosphoprotein 1) (SPP-1) | 0.88 |
| | | | | | | | (Urinary stone protein) (Nephropontin) (Uropontin). | |
| 22 | uniprotKB_sptr | P62937 | PPIA_HUMAN | 18012.497 | 8.34 | 3 | Peptidyl-prolyl cis-trans isomerase A (EC 5.2.1.8) (PPlase A) (Rotamase A) | 8.25 |
| | | | | | | | (Cyclophilin A) (Cyclosporin A-binding protein). | |
| 55 | uniprotKB_sptr | P62937 | PPIA_HUMAN | 18,012.50 | 8.34 | 2 | Peptidyl-prolyl cis-trans isomerase A (EC 5.2.1.8) (PPlase A) (Rotamase A) | 1.69 |
| | | | | | | | (Cyclophilin A) (Cyclosporin A-binding protein). | |
| 21 | uniprotKB_sptr | P30086 | PEBP1_HUMAN | 21056.788 | 7.76 | 3 | Phosphatidylethanolamine-binding protein 1 (PEBP-1) (Prostatic-binding protein) | 4.88 |
| | | | | | | | (HCNPpp) (Neuropolypeptide h3) (Raf kinase inhibitor protein) (RKIP) [Contains: | |
| | | | | | | | Hippocampal cholinergic neurostimulating peptide (HCNP)]. | |
| 51 | uniprotKB_sptr | P00747 | PLMN_HUMAN | 9,019.20 | 6.78 | 2 | Plasminogen precursor (EC 3.4.21.7) [Contains: Plasmin heavy chain A; | 1.22 |
| | | | | | | | Activation peptide; Angiostatin; Plasmin heavy chain A, short form; Plasmin light | |
| | | | | | | | chain B]. | |
| 44 | uniprotKB_sptr | P07602 | SAP_HUMAN | 26308.661 | 5.88 | 2 | Proactivator polypeptide precursor [Contains: Saposin-A (Protein A); Saposin-B- | 1.93 |
| | | | | | | | Val; Saposin-B (Sphingolipid activator protein 1) (SAP-1) (Cerebroside sulfate | |
| | | | | | | | activator) (CSAct) (Dispersin) (Sulfatide/GM1 activator); Saposin-C (Co-beta- | |
| | | | | | | | glucosidase) (A1 activator) (Glucosylceramidase activator) (Sphingolipid activator | |
| | | | | | | | protein 2) (SAP-2); Saposin-D (Protein C) (Component C)]. | |
| 12 | uniprotKB_sptr | P41222_CHAIN_0 | PTGDS_HUMAN | 18698.032 | 8.68 | 6 | Prostaglandin-H2 D-isomerase precursor (EC 5.3.99.2) (Lipocalin-type | 0.27 |
| | | | | | | | prostaglandin-D synthase) (Glutathione-independent PGD synthetase) | |
| | | | | | | | (Prostaglandin-D2 synthase) (PGD2 synthase) (PGDS2) (PGDS) (Beta-trace | |
| | | | | | | | protein) (Cerebrin-28). [CHAIN 0] | |
| 30 | uniprotKB_sptr | P41222_CHAIN_0 | PTGDS_HUMAN | 18,698.03 | 8.68 | 3 | Prostaglandin-H2 D-isomerase precursor (EC 5.3.99.2) (Lipocalin-type | 0.46 |
| | | | | | | | prostaglandin-D synthase) (Glutathione-independent PGD synthetase) | |
| | | | | | | | (Prostaglandin-D2 synthase) (PGD2 synthase) (PGDS2) (PGDS) (Beta-trace | |
| | | | | | | | protein) (Cerebrin-28). [CHAIN 0] | |
| 29 | uniprotKB_sptr | P05109 | S10A8_HUMAN | 10,834.51 | 6.96 | 3 | Protein S100-A8 (S100 calcium-binding protein A8) (Calgranulin-A) (Migration | 0.98 |
| | | | | | | | inhibitory factor-related protein 8) (MRP-8) (Cystic fibrosis antigen) (CFAG) (P8) | |
| | | | | | | | (Leukocyte L1 complex light chain) (Calprotectin L1L subunit) (Urinary stone | |
| | | | | | | | protein band A). | |
| 19 | uniprotKB_sptr | Q6P5S8 | Q6P5S8_HUMAN | 25,772.87 | 6.31 | 2 | Putative uncharacterized protein. | 0.15 |
| 62 | uniprotKB_sptr | A0N5G5 | A0N5G5_HUMAN | 12,766.37 | 9.45 | 2 | Rheumatoid factor D5 light chain (Fragment). | 0.67 |
| 6 | uniprotKB_sptr | P02787 | TRFE_HUMAN | 77049.89 | 6.97 | 17 | Serotransferrin precursor (Transferrin) (Siderophilin) (Beta-1-metal-binding | 1.47 |
| | | | | | | | globulin). | |
| 7 | uniprotKB_sptr | P02787 | TRFE_HUMAN | 77,049.89 | 6.97 | 8 | Serotransferrin precursor (Transferrin) (Siderophilin) (Beta-1-metal-binding | 0.42 |
| | | | | | | | globulin). | |
| 1 | uniprotKB_sptr | P02768 | ALBU_HUMAN | 69366.701 | 5.98 | 33 | Serum albumin precursor. | 1.17 |
| 1 | uniprotKB_sptr | P02768 | ALBU_HUMAN | 69,366.70 | 5.98 | 32 | Serum albumin precursor. | 0.38 |
| 18 | uniprotKB_sptr | Q9UP60 | Q9UP60_HUMAN | 53,088.18 | 6.70 | 3 | SNC73 protein. | 0.30 |
| 29 | uniprotKB_sptr | P00441 | SODC_HUMAN | 16122.002 | 6.04 | 2 | Superoxide dismutase [Cu—Zn](EC 1.15.1.1). | 1.83 |
| 43 | uniprotKB_sptr | P63313 | TYB10_HUMAN | 5025.673 | 6.21 | 2 | Thymosin beta-10. | 4.16 |
| 37 | uniprotKB_sptr | P62328 | TYB4_HUMAN | 7312.177 | 8.84 | 2 | Thymosin beta-4 (T beta 4) (Fx) [Contains: Hematopoietic system regulatory | 2.39 |
| | | | | | | | peptide (Seraspenide)]. | |
| 17 | uniprotKB_sptr | P60174 | TPIS_HUMAN | 26669.495 | 6.90 | 5 | Triosephosphate isomerase (EC 5.3.1.1) (TIM) (Triose-phosphate isomerase). | 3.50 |
| 57 | uniprotKB_sptr | P06753 | TPM3_HUMAN | 28,809.25 | 4.75 | 2 | Tropomyosin alpha-3 chain (Tropomyosin-3) (Tropomyosin gamma) (hTM5). | 2.04 |
| 32 | uniprotKB_sptr | P62988 | UBIQ_HUMAN | 25761.65 | 7.78 | 2 | Ubiquitin. | 1.71 |

TABLE 6
List of regulated proteins in penumbra relative to contralateral hemisphere. Ratio>1 increased in P; Ratio <1 decreased in P

| | | | | | | nb | | Cor [symbol] |
|---|---|---|---|---|---|---|---|---|
| | | | | | | uniqe | | Rati [symbol] |
| Nr. | databank | AC | ID | MW | PI | pept | description | IC/ [symbol] |
| 38 | uniprotKB_sptr | P63104 | 1433Z_HUMAN | 27,745.11 | 4.77 | 3 | 14-3-3 protein zeta/delta (Protein kinase C inhibitor protein 1) (KCIP-1). | 1.51 |
| 57 | uniprotKB_sptr | P60709 | ACTB_HUMAN | 41,004.97 | 5.67 | 2 | Actin, cytoplasmic 1 (Beta-actin). | 1.15 |
| 25 | uniprotKB_sptr | Q13747 | Q13747_HUMAN | 22,828.35 | 6.35 | 8 | Alpha-1 antitrypsin (Fragment). | 1.05 |
| 27 | uniprotKB_sptr | Q13747 | Q13747_HUMAN | 22,828.35 | 6.35 | 7 | Alpha-1 antitrypsin (Fragment). | 0.35 |
| 39 | uniprotKB_sptr | P02763 | A1AG1_HUMAN | 21,560.13 | 5.10 | 2 | Alpha-1-acid glycoprotein 1 precursor (AGP 1) (Orosomucoid-1) (OMD 1). | 1.38 |
| 53 | uniprotKB_sptr | P02763 | A1AG1_HUMAN | 23,539.62 | 5.12 | 2 | Alpha-1-acid glycoprotein 1 precursor (AGP 1) (Orosomucoid-1) (OMD 1). | 0.38 |
| 42 | uniprotKB_sptr | P04217 | A1BG_HUMAN | 51,940.72 | 5.72 | 3 | Alpha-1B-glycoprotein precursor (Alpha-1-B glycoprotein). | 1.11 |
| 24 | uniprotKB_sptr | P01023 | A2MG_HUMAN | 160,796.89 | 5.98 | 7 | Alpha-2-macroglobulin precursor (Alpha-2-M). | 0.68 |
| 47 | uniprotKB_sptr | P01023 | A2MG_HUMAN | 160,796.89 | 5.98 | 4 | Alpha-2-macroglobulin precursor (Alpha-2-M). | 0.36 |
| 55 | uniprotKB_sptr | P06733 | ENOA_HUMAN | 47,037.78 | 7.55 | 3 | Alpha-enolase (EC 4.2.1.11) (2-phospho-D-glycerate hydro-lyase) (Non-neural enolase) | 0.45 |
| | | | | | | | (NNE) (Enolase 1) (Phosphopyruvate hydratase) (C-myc promoter-binding protein) | |
| | | | | | | | (MBP-1) (MPB-1) (Plasminogen-binding protein). | |
| 92 | uniprotKB_sptr | P51693 | APLP1_HUMAN | 72,176.41 | 5.58 | 2 | Amyloid-like protein 1 precursor (APLP) (APLP-1) [Contains: C30]. | 0.86 |
| 91 | uniprotKB_sptr | P01019 | ANGT_HUMAN | 53,777.91 | 6.07 | 2 | Angiotensinogen precursor (Serpin A8) [Contains: Angiotensin-1 (Angiotensin I) (Ang I); | 0.7 [symbol] |
| | | | | | | | Angiotensin-2 (Angiotensin II) (Ang II); Angiotensin-3 (Angiotensin III) (Ang III) (Des- | |
| | | | | | | | Asp[1]-angiotensin II)]. | |
| 51 | uniprotKB_sptr | P01008 | ANT3_HUMAN | 52,691.56 | 6.32 | 3 | Antithrombin-III precursor (ATIII). | 1.1 [symbol] |
| 73 | uniprotKB_sptr | P02652 | APOA2_HUMAN | 8,579.77 | 5.39 | 2 | Apolipoprotein A-II precursor (Apo-AII) (ApoA-II) [Contains: Apolipoprotein A-II(1-76)]. | 1.2 [symbol] |
| 96 | uniprotKB_sptr | P02652 | APOA2_HUMAN | 8,579.77 | 5.39 | 2 | Apolipoprotein A-II precursor (Apo-AII) (ApoA-II) [Contains: Apolipoprotein A-II(1-76)]. | 1.0 [symbol] |
| 61 | uniprotKB_sptr | P02649_CHAIN_0 | APOE_HUMAN | 34,236.69 | 5.65 | 2 | Apolipoprotein E precursor (Apo-E). [CHAIN 0] | 0.3 [symbol] |
| 87 | uniprotKB_sptr | P55087 | AQP4_HUMAN | 34,829.70 | 8.09 | 2 | Aquaporin-4 (AQP-4) (WCH4) (Mercurial-insensitive water channel) (MIWC). | 2.0 [symbol] |
| 68 | uniprotKB_sptr | A6XND9 | A6XND9_HUMAN | 11,748.41 | 6.28 | 2 | Beta-2-microglobulin. | 0.9 [symbol] |
| 88 | uniprotKB_sptr | A6XND9 | A6XND9_HUMAN | 11,748.41 | 6.28 | 2 | Beta-2-microglobulin. | 0.2 [symbol] |
| 70 | uniprotKB_sptr | Q96KN2 | CNDP1_HUMAN | 56,734.20 | 5.24 | 2 | Beta-Ala-His dipeptidase precursor (EC 3.4.13.20) (Carnosine dipeptidase 1) (CNDP | 0.3 [symbol] |
| | | | | | | | dipeptidase 1) (Serum carnosinase) (Glutamate carboxypeptidase-like protein 2). | |
| 80 | uniprotKB_sptr | Q96GW7 | PGCB_HUMAN | 92,811.83 | 4.58 | 2 | Brevican core protein precursor (Brain-enriched hyaluronan-binding protein) (Protein | 0.72 |
| | | | | | | | BEHAB). | |
| 75 | uniprotKB_sptr | Q9BRL5 | Q9BRL5_HUMAN | 16,506.53 | 4.38 | 3 | CALM3 protein. | 1.53 |
| 36 | uniprotKB_sptr | P00915 | CAH1_HUMAN | 28,739.02 | 6.92 | 4 | Carbonic anhydrase 1 (EC 4.2.1.1) (Carbonic anhydrase I) (Carbonate dehydratase I) | 1.09 |
| | | | | | | | (CA-I). | |
| 39 | uniprotKB_sptr | P00915 | CAH1_HUMAN | 28,870.21 | 6.83 | 5 | Carbonic anhydrase 1 (EC 4.2.1.1) (Carbonic anhydrase I) (Carbonate dehydratase I) | 0.71 |
| | | | | | | | (CA-I). | |
| 71 | uniprotKB_sptr | P00918 | CAH2_HUMAN | 29,246.06 | 7.22 | 3 | Carbonic anhydrase 2 (EC 4.2.1.1) (Carbonic anhydrase II) (Carbonate dehydratase II) | 1.77 |
| | | | | | | | (CA-II) (Carbonic anhydrase C). | |
| 60 | uniprotKB_sptr | P13987 | CD59_HUMAN | 14,177.29 | 6.38 | 2 | CD59 glycoprotein precursor (Membrane attack complex inhibition factor) (MACIF) | 1.54 |
| | | | | | | | (MAC-inhibitory protein) (MAC-IP) (Protectin) (MEM43 antigen) (Membrane inhibitor of | |
| | | | | | | | reactive lysis) (MIRL) (20 kDa homologous restriction factor) (HRF-20) (HRF20) (1F5 | |
| | | | | | | | antigen). | |
| 95 | uniprotKB_sptr | P13987 | CD59_HUMAN | 8,961.10 | 5.70 | 2 | CD59 glycoprotein precursor (Membrane attack complex inhibition factor) (MACIF) | 1.19 |
| | | | | | | | (MAC-inhibitory protein) (MAC-IP) (Protectin) (MEM43 antigen) (Membrane inhibitor of | |
| | | | | | | | reactive lysis) (MIRL) (20 kDa homologous restriction factor) (HRF-20) (HRF20) (1F5 | |
| | | | | | | | antigen). | |
| 16 | uniprotKB_sptr | A8K866 | A8K866_HUMAN | 30,777.84 | 5.68 | 11 | cDNA FLJ75790, highly similar to Homo sapiens apolipoprotein A-I (APOA1), mRNA | 0.85 |
| | | | | | | | (Apolipoprotein A-I, isoform CRA_a). | |
| 22 | uniprotKB_sptr | A8K866 | A8K866_HUMAN | 30,777.84 | 5.68 | 8 | cDNA FLJ75790, highly similar to Homo sapiens apolipoprotein A-I (APOA1), mRNA | 0.76 |
| | | | | | | | (Apolipoprotein A-I, isoform CRA_a). | |
| 17 | uniprotKB_sptr | P00450 | CERU_HUMAN | 122,205.23 | 5.46 | 9 | Ceruloplasmin precursor (EC 1.16.3.1) (Ferroxidase). | 0.8 [symbol] |
| 35 | uniprotKB_sptr | P00450 | CERU_HUMAN | 122,205.23 | 5.46 | 5 | Ceruloplasmin precursor (EC 1.16.3.1) (Ferroxidase). | 0.4 [symbol] |
| 64 | uniprotKB_sptr | Q2TU75 | Q2TU75_HUMAN | 52,494.59 | 5.97 | 2 | Clusterin. | 0.4 [symbol] |
| 89 | uniprotKB_sptr | Q14019 | COTL1_HUMAN | 15,944.98 | 5.86 | 2 | Coactosin-like protein. | 2.6 [symbol] |
| 32 | uniprotKB_sptr | P0C0L4 | CO4A_HUMAN | 84,183.35 | 5.38 | 5 | Complement C4-A precursor (Acidic complement C4) [Contains: Complement C4 beta | 1.1 [symbol] |
| | | | | | | | chain; Complement C4-A alpha chain; C4a anaphylatoxin; C4b-A; C4d-A; Complement | |
| | | | | | | | C4 gamma chain]. | |
| 72 | uniprotKB_sptr | P0C0L4 | CO4A_HUMAN | 84,183.35 | 5.38 | 3 | Complement C4-A precursor (Acidic complement C4) [Contains: Complement C4 beta | 0.7 [symbol] |
| | | | | | | | chain; Complement C4-A alpha chain; C4a anaphylatoxin; C4b-A; C4d-A; Complement | |
| | | | | | | | C4 gamma chain]. | |
| 8 | uniprotKB_sptr | A7E236 | A7E236_HUMAN | 187,148.13 | 6.05 | 4 | Complement component 3 (Complement component 3, isoform CRA_b). | 0.72 |
| 12 | uniprotKB_sptr | A7E236 | A7E236_HUMAN | 187,148.13 | 6.05 | 12 | Complement component 3 (Complement component 3, isoform CRA_b). | 0.48 |
| 72 | uniprotKB_sptr | P00751 | CFAB_HUMAN | 83,000.83 | 6.80 | 2 | Complement factor B precursor (EC 3.4.21.47) (C3/C5 convertase) (Properdin factor B) | 1.15 |
| | | | | | | | (Glycine-rich beta glycoprotein) (GBG) (PBF2) [Contains: Complement factor B Ba | |
| | | | | | | | fragment; Complement factor B Bb fragment]. | |
| 93 | uniprotKB_sptr | P00751 | CFAB_HUMAN | 83,000.83 | 6.80 | 2 | Complement factor B precursor (EC 3.4.21.47) (C3/C5 convertase) (Properdin factor B) | 0.36 |
| | | | | | | | (Glycine-rich beta glycoprotein) (GBG) (PBF2) [Contains: Complement factor B Ba | |
| | | | | | | | fragment; Complement factor B Bb fragment]. | |
| 63 | uniprotKB_sptr | P04080 | CYTB_HUMAN | 11,139.59 | 7.91 | 2 | Cystatin-B (Stefin-B) (Liver thiol proteinase inhibitor) (CPI-B). | 2.16 |
| 79 | uniprotKB_sptr | P04080 | CYTB_HUMAN | 11,139.59 | 7.91 | 2 | Cystatin-B (Stefin-B) (Liver thiol proteinase inhibitor) (CPI-B). | 1.81 |
| 49 | uniprotKB_sptr | P01034 | CYTC_HUMAN | 15,799.22 | 9.21 | 2 | Cystatin-C precursor (Cystatin-3) (Neuroendocrine basic polypeptide) (Gamma-trace) | 0.17 |
| | | | | | | | (Post-gamma-globulin). | |
| 57 | uniprotKB_sptr | P01034 | CYTC_HUMAN | 15,799.22 | 9.21 | 3 | Cystatin-C precursor (Cystatin-3) (Neuroendocrine basic polypeptide) (Gamma-trace) | 0.38 |
| | | | | | | | (Post-gamma-globulin). | |
| 67 | uniprotKB_sptr | P21291 | CSRP1_HUMAN | 20,436.21 | 8.99 | 2 | Cysteine and glycine-rich protein 1 (Cysteine-rich protein 1) (CRP1) (CRP). | 4.58 |
| 56 | uniprotKB_sptr | P81605_PEPT_1 | DCD_HUMAN | 4,705.34 | 5.61 | 4 | Dermcidin precursor (Preproteolysin) [Contains: Survival-promoting peptide; DCD-1]. | 0.34 |
| | | | | | | | [PEPTIDE 1]) | |
| 33 | uniprotKB_sptr | Q4VWZ6 | Q4VWZ6_HUMAN | 10,144.58 | 6.74 | 6 | Diazepam binding inhibitor, splice form 1c. | 2.2 [symbol] |
| 102 | uniprotKB_sptr | Q16555 | DPYL2_HUMAN | 62,270.61 | 6.00 | 2 | Dihydropyrimidinase-related protein 2 (DRP-2) (Collapsin response mediator protein 2) | 1.1 [symbol] |
| | | | | | | | (CRMP-2) (N2A3). | |
| 44 | uniprotKB_sptr | Q13822 | ENPP2_HUMAN | 105,210.85 | 8.54 | 4 | Ectonucleotide pyrophosphatase/phosphodiesterase family member 2 precursor (EC | 0.4 [symbol] |
| | | | | | | | 3.1.4.39) (E-NPP 2) (Extracellular lysophospholipase D) (LysoPLD) (Autotaxin). | |
| 30 | uniprotKB_sptr | Q53HR3 | Q53HR3_HUMAN | 47,140.92 | 7.57 | 2 | Enolase (EC 4.2.1.11) (Fragment). | 1.8 [symbol] |
| 26 | uniprotKB_sptr | P02671 | FIBA_HUMAN | 94,973.07 | 5.75 | 7 | Fibrinogen alpha chain precursor [Contains: Fibrinopeptide A]. | 2.9 [symbol] |
| 23 | uniprotKB_sptr | P02671 | FIBA_HUMAN | 94,973.07 | 5.75 | 7 | Fibrinogen alpha chain precursor [Contains: Fibrinopeptide A]. | 1.0 [symbol] |
| 68 | uniprotKB_sptr | P04075 | ALDOA_HUMAN | 39,420.03 | 8.49 | 3 | Fructose-bisphosphate aldolase A (EC 4.1.2.13) (Muscle-type aldolase) (Lung cancer | 1.4 [symbol] |
| | | | | | | | antigen NY-LU-1). | |
| 61 | uniprotKB_sptr | P09104 | ENOG_HUMAN | 47,268.59 | 4.95 | 2 | Gamma-enolase (EC 4.2.1.11) (2-phospho-D-glycerate hydro-lyase) (Neural enolase) | 1.19 |
| | | | | | | | (Neuron-specific enolase) (NSE) (Enolase 2). | |
| 38 | uniprotKB_sptr | P14136 | GFAP_HUMAN | 49,505.89 | 5.95 | 4 | Glial fibrillary acidic protein (GFAP). | 0.31 |
| 16 | uniprotKB_sptr | P14136 | GFAP_HUMAN | 49,880.22 | 5.47 | 9 | Glial fibrillary acidic protein (GFAP). | 2.33 |
| 13 | uniprotKB_sptr | P00738 | HPT_HUMAN | 45,205.32 | 6.24 | 12 | Haptoglobin precursor [Contains: Haptoglobin alpha chain; Haptoglobin beta chain]. | 0.86 |
| 10 | uniprotKB_sptr | P00738 | HPT_HUMAN | 27,265.07 | 6.53 | 14 | Haptoglobin precursor [Contains: Haptoglobin alpha chain; Haptoglobin beta chain]. | 0.45 |
| 31 | uniprotKB_sptr | P69905_{—} | HBA_HUMAN | 15,126.36 | 9.07 | 5 | Hemoglobin subunit alpha (Hemoglobin alpha chain) (Alpha-globin). | 0.11 |
| 41 | uniprotKB_sptr | P69905 | HBA_HUMAN | 15,126.36 | 9.07 | 4 | Hemoglobin subunit alpha (Hemoglobin alpha chain) (Alpha-globin). | 0.55 |
| 34 | uniprotKB_sptr | P68871 | HBB_HUMAN | 15,867.22 | 7.26 | 5 | Hemoglobin subunit beta (Hemoglobin beta chain) (Beta-globin) [Contains: LVV- | 0.11 |
| | | | | | | | hemorphin-7]. | |
| 40 | uniprotKB_sptr | P68871 | HBB_HUMAN | 15,998.41 | 7.13 | 5 | Hemoglobin subunit beta (Hemoglobin beta chain) (Beta-globin) [Contains: LVV- | 0.59 |
| | | | | | | | hemorphin-7]. | |
| 20 | uniprotKB_sptr | P02790 | HEMO_HUMAN | 51,676.39 | 6.67 | 8 | Hemopexin precursor (Beta-1B-glycoprotein). | 0.90 |
| 25 | uniprotKB_sptr | P02790 | HEMO_HUMAN | 51,676.39 | 6.67 | 6 | Hemopexin precursor (Beta-1B-glycoprotein). | 0.52 |
| 83 | uniprotKB_sptr | Q16775 | GLO2_HUMAN | 28,860.02 | 7.19 | 2 | Hydroxyacylglutathione hydrolase (EC 3.1.2.6) (Glyoxalase II) (Glx II). | 2.64 |
| 37 | uniprotKB_sptr | P01876 | IGHA1_HUMAN | 37,654.65 | 6.26 | 4 | Ig alpha-1 chain C region. | 0.66 |
| 82 | uniprotKB_sptr | P01877 | IGHA2_HUMAN | 53,776.22 | 6.91 | 2 | Ig alpha-2 chain C region. | 0.51 |
| 27 | uniprotKB_sptr | P01859 | IGHG2_HUMAN | 35,884.65 | 8.01 | 2 | Ig gamma-2 chain C region. | 0.87 |
| 24 | uniprotKB_sptr | P01859 | IGHG2_HUMAN | 35,884.65 | 8.01 | 2 | Ig gamma-2 chain C region. | 0.2 [symbol] |
| 67 | uniprotKB_sptr | P01871 | IGHM_HUMAN | 65,290.86 | 8.57 | 2 | Ig mu chain C region. | 0.5 [symbol] |
| 29 | uniprotKB_sptr | Q6GMW0 | Q6GMW0_HUMAN | 25,772.87 | 6.31 | 4 | IGKV1-5 protein. | 0.7 [symbol] |
| 35 | uniprotKB_sptr | Q16270 | IBP7_HUMAN | 26,443.00 | 8.25 | 4 | Insulin-like growth factor-binding protein 7 precursor (IGFBP-7) (IBP-7) (IGF-binding | 0.0 [symbol] |
| | | | | | | | protein 7) (MAC25 protein) (Prostacyclin-stimulating factor) (PGI2-stimulating factor) | |
| | | | | | | | (IGFBP-rP1). | |
| 31 | uniprotKB_sptr | Q92876 | KLK6_HUMAN | 26,855.73 | 7.57 | 5 | Kallikrein-6 precursor (EC 3.4.21.—) (Protease M) (Neurosin) (Zyme) (SP59) (Serine | 0.4 [symbol] |
| | | | | | | | protease 9) (Serine protease 18). | |
| 11 | uniprotKB_sptr | P13645 | K1C10_HUMAN | 59,510.71 | 5.18 | 10 | Keratin, type I cytoskeletal 10 (Cytokeratin-10) (CK-10) (Keratin-10) (K10). | 0.7 [symbol] |
| 11 | uniprotKB_sptr | P13645 | K1C10_HUMAN | 59,510.71 | 5.18 | 8 | Keratin, type I cytoskeletal 10 (Cytokeratin-10) (CK-10) (Keratin-10) (K10). | 0.53 |
| 19 | uniprotKB_sptr | P02533 | K1C14_HUMAN | 51,490.33 | 5.13 | 3 | Keratin, type I cytoskeletal 14 (Cytokeratin-14) (CK-14) (Keratin-14) (K14). | 0.59 |
| 26 | uniprotKB_sptr | P02533 | K1C14_HUMAN | 51,621.52 | 5.13 | 2 | Keratin, type I cytoskeletal 14 (Cytokeratin-14) (CK-14) (Keratin-14) (K14). | 0.68 |
| 12 | uniprotKB_sptr | P08779 | K1C16_HUMAN | 51,267.84 | 5.03 | 5 | Keratin, type I cytoskeletal 16 (Cytokeratin-16) (CK-16) (Keratin-16) (K16). | 0.60 |
| 10 | uniprotKB_sptr | P35527 | K1C9_HUMAN | 62,129.47 | 5.24 | 12 | Keratin, type I cytoskeletal 9 (Cytokeratin-9) (CK-9) (Keratin-9) (K9). | 0.76 |
| 8 | uniprotKB_sptr | P35527 | K1C9_HUMAN | 62,129.47 | 5.24 | 12 | Keratin, type I cytoskeletal 9 (Cytokeratin-9) (CK-9) (Keratin-9) (K9). | 0.79 |
| 5 | uniprotKB_sptr | P04264 | K2C1_HUMAN | 66,017.70 | 8.45 | 18 | Keratin, type II cytoskeletal 1 (Cytokeratin-1) (CK-1) (Keratin-1) (K1) (67 kDa cytokeratin) | 0.73 |
| | | | | | | | (Hair alpha protein). | |
| 7 | uniprotKB_sptr | P04264 | K2C1_HUMAN | 66,017.70 | 8.45 | 15 | Keratin, type II cytoskeletal 1 (Cytokeratin-1) (CK-1) (Keratin-1) (K1) (67 kDa cytokeratin) | 0.63 |
| | | | | | | | (Hair alpha protein). | |
| 14 | uniprotKB_sptr | P35908 | K22E_HUMAN | 65,865.35 | 8.35 | 5 | Keratin, type II cytoskeletal 2 epidermal (Cytokeratin-2e) (K2e) (CK 2e) (keratin-2). | 1.08 |
| 9 | uniprotKB_sptr | P35908 | K22E_HUMAN | 65,865.35 | 8.35 | 7 | Keratin, type II cytoskeletal 2 epidermal (Cytokeratin-2e) (K2e) (CK 2e) (keratin-2). | 0.33 |
| 18 | uniprotKB_sptr | P13647 | K2C5_HUMAN | 62,378.37 | 8.14 | 4 | Keratin, type II cytoskeletal 5 (Cytokeratin-5) (CK-5) (Keratin-5) (K5) (58 kDa | 0.69 |
| | | | | | | | cytokeratin). | |
| 20 | uniprotKB_sptr | P13647 | K2C5_HUMAN | 62,378.37 | 8.14 | 3 | Keratin, type II cytoskeletal 5 (Cytokeratin-5) (CK-5) (Keratin-5) (K5) (58 kDa | 0.58 |
| | | | | | | | cytokeratin). | |
| 15 | uniprotKB_sptr | P02538 | K2C6A_HUMAN | 60,044.97 | 8.38 | 5 | Keratin, type II cytoskeletal 6A (Cytokeratin-6A) (CK 6A) (K6a keratin) (Cytokeratin-6D) | 0.58 |
| | | | | | | | (CK 6D). | |
| 65 | uniprotKB_sptr | P01042 | KNG1_HUMAN | 69,896.73 | 6.29 | 2 | Kininogen-1 precursor (Alpha-2-thiol proteinase inhibitor) [Contains: Kininogen-1 heavy | 3.9 [symbol] |
| | | | | | | | chain; Bradykinin (Kallidin I); Lysyl-bradykinin (Kallidin II); Kininogen-1 light chain; Low | |
| | | | | | | | molecular weight growth-promoting factor]. | |
| 30 | uniprotKB_sptr | A2NUT2 | A2NUT2_HUMAN | 25,020.97 | 8.45 | 3 | Lambda-chain precursor (AA −20 to 215). | 0.8 [symbol] |
| 46 | uniprotKB_sptr | A2NUT2 | A2NUT2_HUMAN | 24,960.80 | 5.40 | 2 | Lambda-chain precursor (AA −20 to 215). | 0.3 [symbol] |
| 43 | uniprotKB_sptr | P61626 | LYSC_HUMAN | 16,537.02 | 9.50 | 3 | Lysozyme C precursor (EC 3.2.1.17) (1,4-beta-N-acetylmuramidase C). | 2.2 [symbol] |
| 81 | uniprotKB_sptr | P40925 | MDHC_HUMAN | 36,426.13 | 7.61 | 2 | Malate dehydrogenase, cytoplasmic (EC 1.1.1.37) (Cytosolic malate dehydrogenase). | 1.1 [symbol] |
| 62 | uniprotKB_sptr | P02795 | MT2_HUMAN | 6,042.16 | 8.38 | 2 | Metallothionein-2 (MT-2) (Metallothionein-II) (MT-II) (Metallothionein-2A). | 6.0 [symbol] |
| 60 | uniprotKB_sptr | P02795 | MT2_HUMAN | 6,042.16 | 8.38 | 3 | Metallothionein-2 (MT-2) (Metallothionein-II) (MT-II) (Metallothionein-2A). | 4.7 [symbol] |
| 62 | uniprotKB_sptr | P25713 | MT3_HUMAN | 6,926.97 | 5.00 | 3 | Metallothionein-3 (MT-3) (Metallothionein-III) (MT-III) (Growth inhibitory factor) (GIF) | 4.5 [symbol] |
| | | | | | | | (GIFB). | |
| 76 | uniprotKB_sptr | P78559 | MAP1A_HUMAN | ########## | 4.86 | 2 | Microtubule-associated protein 1A (MAP 1A) (Proliferation-related protein p80) [Contains: | 1.56 |
| | | | | | | | MAP1 light chain LC2]. | |
| 58 | uniprotKB_sptr | P46821 | MAP1B_HUMAN | 192,380.44 | 4.50 | 3 | Microtubule-associated protein 1B (MAP 1B) [Contains: MAP1 light chain LC1]. | 1.83 |
| 70 | uniprotKB_sptr | P02686_ISOFORM_3 | MBP_HUMAN | 21,493.21 | 11.45 | 3 | Myelin basic protein (MBP) (Myelin A1 protein) (Myelin membrane encephalitogenic | 1.60 |
| | | | | | | | protein). [ISOFORM 3] | |
| 90 | uniprotKB_sptr | P20916 | MAG_HUMAN | 69,040.49 | 4.97 | 2 | Myelin-associated glycoprotein precursor (Siglec-4a). | 0.98 |
| 53 | uniprotKB_sptr | P02144 | MYG_HUMAN | 17,052.62 | 8.02 | 2 | Myoglobin. | 3.09 |
| 29 | uniprotKB_sptr | O94760 | DDAH1_HUMAN | 31,121.78 | 5.64 | 6 | N(G),N(G)-dimethylarginine dimethylaminohydrolase 1 (EC 3.5.3.18) | 2.21 |
| | | | | | | | (Dimethylargininase-1) (Dimethylarginine dimethylaminohydrolase 1) (DDAHI) (DDAH-1). | |
| 44 | uniprotKB_sptr | P07197 | NFM_HUMAN | 102,316.84 | 4.90 | 5 | Neurofilament medium polypeptide (NF-M) (Neurofilament triplet M protein) (160 kDa | 2.62 |
| | | | | | | | neurofilament protein) (Neurofilament 3). | |
| 47 | uniprotKB_sptr | P59665 | DEF1_HUMAN | 10,244.95 | 6.54 | 2 | Neutrophil defensin 1 precursor (HNP-1) (HP-1) (HP1) (Defensin, alpha 1) [Contains: HP | 0.17 |
| | | | | | | | 1-56; Neutrophil defensin 2 (HNP-2) (HP-2) (HP2)]. | |
| 98 | uniprotKB_sptr | P10451 | OSTP_HUMAN | 33,016.56 | 4.59 | 2 | Osteopontin precursor (Bone sialoprotein 1) (Secreted phosphoprotein 1) (SPP-1) | 0.52 |
| | | | | | | | (Urinary stone protein) (Nephropontin) (Uropontin). | |
| 15 | uniprotKB_sptr | P62937 | PPIA_HUMAN | 18,012.50 | 8.34 | 10 | Peptidyl-prolyl cis-trans isomerase A (EC 5.2.1.8) (PPlase A) (Rotamase A) (Cyclophilin | 3.28 |
| | | | | | | | A) (Cyclosporin A-binding protein). | |
| 74 | uniprotKB_sptr | Q06830 | PRDX1_HUMAN | 22,110.36 | 8.59 | 3 | Peroxiredoxin-1 (EC 1.11.1.15) (Thioredoxin peroxidase 2) (Thioredoxin-dependent | 1.2 [symbol] |
| | | | | | | | peroxide reductase 2) (Proliferation-associated gene protein) (PAG) (Natural killer cell- | |
| | | | | | | | enhancing factor A) (NKEF-A). | |
| 51 | uniprotKB_sptr | P30041 | PRDX6_HUMAN | 208,794.31 | 5.37 | 4 | Peroxiredoxin-6 (EC 1.11.1.15) (Antioxidant protein 2) (1-Cys peroxiredoxin) (1-Cys | 1.6 [symbol] |
| | | | | | | | PRX) (Acidic calcium-independent phospholipase A2) (EC 3.1.1.—) (aiPLA2) (Non- | |
| | | | | | | | selenium glutathione peroxidase) (EC 1.11.1.7) (NSGPx) (24 kDa protein) (Liver 2D page | |
| | | | | | | | spot 40) (Red blood cells page spot 12). | |
| 19 | uniprotKB_sptr | P30086 | PEBP1_HUMAN | 21,056.79 | 7.76 | 7 | Phosphatidylethanolamine-binding protein 1 (PEBP-1) (Prostatic-binding protein) | |
| | | | | | | | (HCNPpp) (Neuropolypeptide h3) (Raf kinase inhibitor protein) (RKIP) [Contains: | 3.3 [symbol] |
| | | | | | | | Hippocampal cholinergic neurostimulating peptide (HCNP)]. | |
| 28 | uniprotKB_sptr | P36955 | PEDF_HUMAN | 46,342.31 | 6.12 | 6 | Pigment epithelium-derived factor precursor (PEDF) (Serpin-F1) (EPC-1). | 0.1 [symbol] |
| 54 | uniprotKB_sptr | P00747 | PLMN_HUMAN | 63,245.42 | 6.99 | 2 | Plasminogen precursor (EC 3.4.21.7) [Contains: Plasmin heavy chain A; Activation | 2.5 [symbol] |
| | | | | | | | peptide; Angiostatin; Plasmin heavy chain A, short form; Plasmin light chain B]. | |
| 40 | uniprotKB_sptr | P02775 | SCYB7_HUMAN | 6,982.36 | 9.64 | 3 | Platelet basic protein precursor (PBP) (C—X—C motif chemokine 7) (Small-inducible | 3.13 |
| | | | | | | | cytokine B7) (Leukocyte-derived growth factor) (LDGF) (Macrophage-derived growth | |
| | | | | | | | factor) (MDGF) [Contains: Connective tissue-activating peptide III (CTAP-III) (Low-affinity | |
| | | | | | | | platelet factor IV) (LA-PF4); TC-2; Connective tissue-activating peptide III(1-81) (CTAP- | |
| | | | | | | | III(1-81)); Beta-thromboglobulin (Beta-TG); Neutrophil-activating peptide 2(74) (NAP- | |
| | | | | | | | 2(74)); Neutrophil-activating peptide 2(73) (NAP-2(73)); Neutrophil-activating peptide 2 | |
| | | | | | | | (NAP-2); TC-1; Neutrophil-activating peptide 2(1-66) (NAP-2(1-66)); Neutrophil- | |
| | | | | | | | activating peptide 2(1-63) (NAP-2(1-63))]. | |
| 46 | uniprotKB_sptr | P20742 | PZP_HUMAN | 140,363.79 | 5.95 | 2 | Pregnancy zone protein precursor. | 0.78 |
| 45 | uniprotKB_sptr | P07602 | SAP_HUMAN | 26,308.66 | 5.88 | 3 | Proactivator polypeptide precursor [Contains: Saposin-A (Protein A); Saposin-B-Val; | 1.54 |
| | | | | | | | Saposin-B (Sphingolipid activator protein 1) (SAP-1) (Cerebroside sulfate activator) | |
| | | | | | | | (CSAct) (Dispersin) (Sulfatide/GM1 activator); Saposin-C (Co-beta-glucosidase) (A1 | |
| | | | | | | | activator) (Glucosylceramidase activator) (Sphingolipid activator protein 2) (SAP-2); | |
| | | | | | | | Saposin-D (Protein C) (Component C)]. | |
| 50 | uniprotKB_sptr | P07602 | SAP_HUMAN | 26,308.66 | 5.88 | 4 | Proactivator polypeptide precursor [Contains: Saposin-A (Protein A); Saposin-B-Val; | 1.70 |
| | | | | | | | Saposin-B (Sphingolipid activator protein 1) (SAP-1) (Cerebroside sulfate activator) | |
| | | | | | | | (CSAct) (Dispersin) (Sulfatide/GM1 activator); Saposin-C (Co-beta-glucosidase) (A1 | |
| | | | | | | | activator) (Glucosylceramidase activator) (Sphingolipid activator protein 2) (SAP-2); | |
| | | | | | | | Saposin-D (Protein C) (Component C)]. | |
| 66 | uniprotKB_sptr | P07737 | PROF1_HUMAN | 15,054.23 | 8.79 | 2 | Profilin-1 (Profilin I). | 3.81 |
| 33 | uniprotKB_sptr | P41222_CHAIN_0 | PTGDS_HUMAN | 18,698.03 | 8.68 | 5 | Prostaglandin-H2 D-isomerase precursor (EC 5.3.99.2) (Lipocalin-type prostaglandin-D | 0.2 [symbol] |
| | | | | | | | synthase) (Glutathione-independent PGD synthetase) (Prostaglandin-D2 synthase) | |
| | | | | | | | (PGD2 synthase) (PGDS2) (PGDS) (Beta-trace protein) (Cerebrin-28). [CHAIN 0] | |
| 17 | uniprotKB_sptr | P41222_CHAIN_0 | PTGDS_HUMAN | 18,698.03 | 8.68 | 9 | Prostaglandin-H2 D-isomerase precursor (EC 5.3.99.2) (Lipocalin-type prostaglandin-D | 0.6 [symbol] |
| | | | | | | | synthase) (Glutathione-independent PGD synthetase) (Prostaglandin-D2 synthase) | |
| | | | | | | | (PGD2 synthase) (PGDS2) (PGDS) (Beta-trace protein) (Cerebrin-28). [CHAIN 0] | |
| 56 | uniprotKB_sptr | P00734 | THRB_HUMAN | 70,008.83 | 5.69 | 2 | Prothrombin precursor (EC 3.4.21.5) (Coagulation factor II) [Contains: Activation peptide | 2.6 [symbol] |
| | | | | | | | fragment 1; Activation peptide fragment 2; Thrombin light chain; Thrombin heavy chain]. | |
| 18 | uniprotKB_sptr | Q6N030 | Q6N030_HUMAN | 57,019.61 | 8.48 | 2 | Putative uncharacterized protein DKFZp686I15212. | 0.3 [symbol] |
| 34 | uniprotKB_sptr | P14618_ISOFORM_M1 | KPYM_HUMAN | 58,062.08 | 7.99 | 5 | Pyruvate kinase isozymes M1/M2 (EC 2.7.1.40) (Pyruvate kinase muscle isozyme) | 1.6 [symbol] |
| | | | | | | | (Pyruvate kinase 2/3) (Cytosolic thyroid hormone-binding protein) (CTHBP) (THBP1). | |
| | | | | | | | [ISOFORM M1] | |
| 59 | uniprotKB_sptr | A0N5G5 | A0N5G5_HUMAN | 12,766.37 | 9.45 | 2 | Rheumatoid factor D5 light chain (Fragment). | 1.06 |
| 101 | uniprotKB_sptr | A0N5G5 | A0N5G5_HUMAN | 12,766.37 | 9.45 | 2 | Rheumatoid factor D5 light chain (Fragment). | 0.53 |
| 85 | uniprotKB_sptr | P05060 | SCG1_HUMAN | 78,246.25 | 5.03 | 2 | Secretogranin-1 precursor (Secretogranin I) (SgI) (Chromogranin-B) (CgB) [Contains: | 1.29 |
| | | | | | | | GAWK peptide; CCB peptide]. | |
| 69 | uniprotKB_sptr | Q13228 | SBP1_HUMAN | 52,390.99 | 6.03 | 2 | Selenium-binding protein 1 (56 kDa selenium-binding protein) (SP56). | 1.42 |
| 6 | uniprotKB_sptr | P02787 | TRFE_HUMAN | 77,049.89 | 6.97 | 23 | Serotransferrin precursor (Transferrin) (Siderophilin) (Beta-1-metal-binding globulin). | 0.91 |
| 5 | uniprotKB_sptr | P02787_CHAIN_0 | TRFE_HUMAN | 76,959.79 | 7.16 | 29 | Serotransferrin precursor (Transferrin) (Siderophilin) (Beta-1-metal-binding globulin). | 0.36 |
| | | | | | | | [CHAIN 0] | |
| 48 | uniprotKB_sptr | Q9H299 | SH3L3_HUMAN | 10,437.72 | 5.03 | 3 | SH3 domain-binding glutamic acid-rich-like protein 3 (SH3 domain-binding protein 1) | 1.88 |
| | | | | | | | (SH3BP-1). | |
| 84 | uniprotKB_sptr | Q9H299 | SH3L3_HUMAN | 23,838.08 | 9.43 | 2 | SH3 domain-binding glutamic acid-rich-like protein 3 (SH3 domain-binding protein 1) | 1.76 |
| | | | | | | | (SH3BP-1). | |
| 52 | uniprotKB_sptr | O75368 | SH3L1_HUMAN | 12,774.25 | 5.53 | 4 | SH3 domain-binding glutamic acid-rich-like protein. | 2.77 |
| 48 | uniprotKB_sptr | Q01082 | SPTB2_HUMAN | 274,609.33 | 5.40 | 4 | Spectrin beta chain, brain 1 (Spectrin, non-erythroid beta chain 1) (Beta-II spectrin) | 2.25 |
| | | | | | | | (Fodrin beta chain). | |
| 42 | uniprotKB_sptr | P16949 | STMN1_HUMAN | 17,336.53 | 5.94 | 4 | Stathmin (Phosphoprotein p19) (pp19) (Oncoprotein 18) (Op18) (Leukemia-associated | 2.15 |
| | | | | | | | phosphoprotein p18) (pp17) (Prosolin) (Metablastin) (Protein Pr22). | |
| 49 | uniprotKB_sptr | P00441 | SODC_HUMAN | 16,122.00 | 6.04 | 4 | Superoxide dismutase [Cu—Zn] (EC 1.15.1.1). | 1.88 |
| 94 | uniprotKB_sptr | P10599 | THIO_HUMAN | 9,451.99 | 6.56 | 2 | Thioredoxin (Trx) (ATL-derived factor) (ADF) (Surface-associated sulphydryl protein) | 1.8 [symbol] |
| | | | | | | | (SASP). | |
| 77 | uniprotKB_sptr | P04216 | THY1_HUMAN | 12,553.22 | 9.33 | 2 | Thy-1 membrane glycoprotein precursor (Thy-1 antigen) (CD90 antigen) (CDw90). | 1.3 [symbol] |
| 45 | uniprotKB_sptr | P60174 | TPIS_HUMAN | 26,669.50 | 6.90 | 4 | Triosephosphate isomerase (EC 5.3.1.1) (TIM) (Triose-phosphate isomerase). | 1.4 [symbol] |
| 99 | uniprotKB_sptr | P09936 | UCHL1_HUMAN | 24,824.35 | 5.45 | 2 | Ubiquitin carboxyl-terminal hydrolase isozyme L1 (EC 3.4.19.12) (EC 6.—.—.—) (UCH-L1) | 2.0 [symbol] |
| | | | | | | | (Ubiquitin thioesterase L1) (Neuron cytoplasmic protein 9.5) (PGP 9.5) (PGP9.5). | |
| 69 | uniprotKB_sptr | P62988 | UBIQ_HUMAN | 25,761.65 | 7.78 | 2 | Ubiquitin. | 2.1 [symbol] |
| 36 | uniprotKB_sptr | P62988 | UBIQ_HUMAN | 61,523.80 | 7.25 | 5 | Ubiquitin. | 2.7 [symbol] |
| 74 | uniprotKB_sptr | P02774 | VTDB_HUMAN | 52,963.66 | 5.47 | 2 | Vitamin D-binding protein precursor (DBP) (Group-specific component) (Gc-globulin) | 0.8 [symbol] |
| | | | | | | | (VDB). | |
| 73 | uniprotKB_sptr | P25311 | ZA2G_HUMAN | 34,244.70 | 5.85 | 3 | Zinc-alpha-2-glycoprotein precursor (Zn-alpha-2-glycoprotein) (Zn-alpha-2-GP). | 0.6 [symbol] |

[symbol]

indicates data missing or illegible when filed

Several proteins such as S100B, glial fibrillary acidic protein (GFAP), and myelin basic protein (MBP), have been already reported to be associated with stroke or other brain pathologies (25-27). The S100B protein was actually identified in Expb but with only one unique peptide (Phenyx peptide score of 11.67). Its IC/P ratio was 3.38. GSTP1, a protein which was initially found increased in post-mortem CSF (15, 20), exhibited an IC/P ratio of 2.79 in Expa. Several peroxiredoxins were also increased in IC samples as reported in Table 7.

TABLE 7
Increased ratios IC/P in microdialysis samples.

| | Ratio IC/P | Ratio IC/P |
|---|---|---|
| Protein description | (Expₐ) | (Exp_{b}) |
| Acyl-CoA-binding protein | 1.95 | 2.67 |
| Beta-2-microglobulin precursor | 1.49 | 2.09 |
| Coactosin-like protein | 1.72 | 2.04 |
| Complement C4-A precursor | 2.50 | 1.10 |
| Cystatin-B | | 2.68 |
| Cysteine and glycine-rich protein 1 | 3.33 | 2.88 |
| Fatty acid-binding protein, brain | 2.65 | |
| Fibrinogen alpha chain precursor | 2.97 | 0.61 |
| Glutathione S-transferase P | 2.79 | |
| Heterogeneous nuclear ribonucleoprotein G | 2.35 | |
| Metallothionein-3 | 2.10 | 2.79 |
| Myelin basic protein [ISOFORM 3] | 1.71 | 3.11 |
| Neutrophil defensin 1 precursor | | 2.45 |
| Paralemmin | 3.52 | |
| Peptidyl-prolyl cis-trans isomerase A | 2.45 | 1.60 |
| Peroxiredoxin-2 | 2.72 | |
| Peroxiredoxin-6 | 2.15 | 2.16 |
| Phosphatidylethanolamine-binding protein 1 | 2.06 | 1.60 |
| Plasma retinol-binding protein precursor | 2.83 | 1.63 |
| Plasminogen precursor | 2.27 | |
| Platelet basic protein precursor | 2.51 | 0.85 |
| Profilin-1 | 2.40 | 0.91 |
| SH3 domain-binding glutamic acid-rich-like protein | 2.17 | 1.92 |
| Thioredoxin | 2.17 | |
| Ubiquitin | 2.09 | 1.50 |

Empty cases derive from the lack of the protein identification/quantitation in the studied sample. The values reported in bold indicate increased ratios (i.e., superior to 2) for both patients (i.e., patient a and b).

Although it was not reported in the table because of ratio value below the cut-off, PRDX1 was measured at a ratio of 1.93 in Expb. In the comparison of P and CT microdialysis samples, PRDX1 and peroxiredoxin-6 (PRDX6) were respectively measured with ratios of 1.24 and 1.69 in Expf.

TABLE 8
Increased ratios IC/CT in microdialysis samples.

| | Ratio IC/CT | Ratio IC/CT |
|---|---|---|
| Protein description | (Exp_{c}) | (Exp_{d}) |
| Acyl-CoA-binding protein | 12.53 | |
| Apolipoprotein A-II precursor | | 2.28 |
| Apolipoprotein A-IV precursor | | 3.79 |
| Carbonic anhydrase 1 | 0.29 | 4.78 |
| Carbonic anhydrase 2 | | 3.18 |
| Chitinase-3-like protein 1 precursor | 2.44 | |
| Cofilin-1 | | 2.00 |
| Cystatin-B | | 2.10 |
| Fibrinogen alpha chain precursor | 0.46 | 2.33 |
| Flavin reductase | | 2.22 |
| Glial fibrillary acidic protein | 4.13 | 0.37 |
| Hemoglobin subunit alpha | | 3.21 |
| Histone H1.2 | | 2.97 |
| Histone H1.5 | | 2.03 |
| Lysozyme C precursor | | 2.75 |
| N(G),N(G)-dimethylarginine | 6.31 | |
| dimethylaminohydrolase 1 | | |
| Neurofilament medium polypeptide | 4.68 | |
| Neutrophil defensin 1 precursor | | 4.23 |
| Peptidyl-prolyl cis-trans isomerase A | 8.25 | 1.69 |
| Phosphatidylethanolamine-binding protein 1 | 4.88 | |
| Thymosin beta-10 | 4.16 | |
| Thymosin beta-4 | 2.39 | |
| Triosephosphate isomerase | 3.50 | |
| Tropomyosin alpha-3 chain | | 2.04 |

Empty cases derive from the lack of the protein identification/quantitation in the studied sample.

TABLE 9
Increased ratios P/CT in microdialysis samples.

| | Ratio P/CT | Ratio P/CT |
|---|---|---|
| Protein description | (Expₑ) | (Exp_{f}) |
| Aquaporin-4 | | 2.00 |
| Coactosin-like protein | | 2.64 |
| Cystatin-B | 2.16 | 1.81 |
| Cysteine and glycine-rich protein 1 | | 4.58 |
| Diazepam binding inhibitor, splice form 1c | | 2.21 |
| Fibrinogen alpha chain precursor | 2.98 | 1.05 |
| Glial fibrillary acidic protein | 0.31 | 2.33 |
| Hydroxyacylglutathione hydrolase | | 2.64 |
| Kininogen-1 precursor | 3.97 | |
| Lysozyme C precursor | 2.21 | |
| Metallothionein-2 | 6.02 | 4.73 |
| Metallothionein-3 | | 4.57 |
| Myoglobin | 3.09 | |
| N(G),N(G)-dimethylarginine | | 2.21 |
| dimethylaminohydrolase 1 | | |
| Neurofilament medium polypeptide | | 2.62 |
| Peptidyl-prolyl cis-trans isomerase A | | 3.28 |
| Phosphatidylethanolamine-binding protein 1 | | 3.35 |
| Plasminogen precursor | 2.52 | |
| Platelet basic protein precursor | 3.13 | |
| Profilin-1 | 3.81 | |
| Prothrombin precursor | 2.64 | |
| SH3 domain-binding glutamic acid-rich-like protein | | 2.77 |
| Spectrin beta chain, brain 1 | | 2.25 |
| Stathmin | | 2.15 |
| Ubiquitin carboxyl-terminal hydrolase isozyme L1 | | 2.06 |
| Ubiquitin | 2.16 | 2.76 |

Empty cases derive from the lack of the protein identification/quantitation in the studied sample. The values reported in bold indicate increased ratios (i.e., superior to 2) for both patients (i.e., patient e and f).

The proteins with ratio inferior to 0.5 are reported in Tables 10-12:

TABLE 10
Decreased ratios IC/P in microdialysis samples.

| | Ratio IC/P | Ratio IC/P |
|---|---|---|
| Protein description | (Expₐ) | (Exp_{b}) |
| Alpha-1-acid glycoprotein 1 precursor | 0.48 | 0.47 |
| Alpha-1-antitrypsin precursor | 0.61 | 0.40 |
| Alpha-1B-glycoprotein precursor | | 0.46 |
| Carbonic anhydrase 1 | 0.45 | 1.60 |
| Ceruloplasmin precursor | | 0.44 |
| Fibrinogen beta chain precursor | | 0.46 |
| Fibrinogen gamma chain precursor | | 0.41 |
| Haptoglobin precursor | 0.46 | 0.36 |
| Hemoglobin subunit alpha | 0.34 | 0.70 |
| Hemoglobin subunit beta | 0.37 | 0.80 |
| Ig kappa chain C region | 0.51 | 0.28 |
| Keratin, type I cytoskeletal 10 | 0.72 | 0.39 |
| Serotransferrin precursor | 0.42 | 0.50 |
| Serum albumin precursor | 0.40 | 0.33 |

Empty cases derive from the lack of the protein identification/quantitation in the studied sample. The values reported in bold indicate increased ratios (i.e., inferior to 0.5) for both patients (i.e., patients a and b).

TABLE 11
Decreased ratios IC/CT in microdialysis samples.

| | Ratio IC/CT | Ratio IC/CT |
|---|---|---|
| Protein description | (Exp_{c}) | (Exp_{d}) |
| Alpha-1-antitrypsin precursor | | 0.49 |
| Alpha-2-macroglobulin precursor | | 0.36 |
| Beta-2-microglobulin precursor | 0.80 | 0.44 |
| Carbonic anhydrase 1 | 0.29 | 4.68 |
| Chromogranin-A precursor | 0.29 | |
| Complement C3 precursor | | 0.43 |
| Cystatin-C precursor | 0.62 | 0.03 |
| Dermcidin precursor | 0.85 | 0.35 |
| Fibrinogen alpha chain precursor | 0.46 | 2.33 |
| Fibrinogen beta chain precursor | | 0.38 |
| Glial fibrillary acidic protein | 4.13 | 0.37 |
| Haptoglobin precursor | 0.83 | 0.14 |
| Ig gamma-2 chain C region | | 0.18 |
| Keratin, type I cytoskeletal 10 | 0.99 | 0.48 |
| Keratin, type II cytoskeletal 2 epidermal | 0.97 | 0.43 |
| Lambda-chain precursor | | 0.21 |
| Myelin basic protein | | 0.30 |
| Prostaglandin-H2 D-isomerase precursor | 0.27 | 0.46 |
| Putative uncharacterized protein | | 0.15 |
| Serotransferrin precursor | 1.47 | 0.42 |
| Serum albumin precursor | 1.17 | 0.38 |
| SNC73 protein | | 0.30 |

Empty cases derive from the lack of the protein identification/quantitation in the studied sample. The values reported in bold indicate increased ratios (i.e., inferior to 0.5) for both patients (i.e., patients c and d).

TABLE 12
Decreased ratios P/CT in microdialysis samples.

| | Ratio | Ratio |
|---|---|---|
| | P/CT | P/CT |
| Protein description | (Expₑ) | (Exp_{f}) |
| Alpha-1-antitrypsin | 1.05 | 0.35 |
| Alpha-1-acid glycoprotein 1 precursor | 1.38 | 0.38 |
| Alpha-2-macroglobulin precursor | 0.68 | 0.36 |
| Alpha-enolase | 0.45 | |
| Apolipoprotein E precursor | 0.30 | |
| Beta-2-microglobulin | 0.92 | 0.25 |
| Beta-Ala-His dipeptidase precursor | 0.32 | |
| Ceruloplasmin precursor | | 0.45 |
| Clusterin | 0.49 | |
| Complement component 3 | 0.72 | 0.48 |
| Complement factor B precursor | 1.15 | 0.36 |
| Cystatin-C precursor | 0.17 | 0.38 |
| Dermcidin precursor | | 0.34 |
| Ectonucleotide pyrophosphatase/ | 0.46 | |
| phosphodiesterase family member 2 precursor | | |
| Glial fibrillary acidic protein | 0.31 | 2.33 |
| Haptoglobin precursor | | 0.45 |
| Hemoglobin subunit alpha | 0.11 | 0.55 |
| Hemoglobin subunit beta | 0.11 | 0.59 |
| Ig gamma-2 chain C region | 0.87 | 0.22 |
| Insulin-like growth factor-binding protein 7 precursor | 0.02 | |
| Kallikrein-6 precursor | | 0.41 |
| Keratin, type II cytoskeletal 2 epidermal | 1.08 | 0.33 |
| Lambda-chain precursor | 0.89 | 0.35 |
| Neutrophil defensin 1 precursor | 0.17 | |
| Pigment epithelium-derived factor precursor | 0.15 | |
| Prostaglandin-H2 D-isomerase precursor | 0.20 | 0.64 |
| Putative uncharacterized protein DKFZp686I15212 | | 0.37 |
| Serotransferrin precursor | 0.91 | 0.36 |

Empty cases derive from the lack of the protein identification/quantitation in the studied sample. The values reported in bold indicate increased ratios (i.e., inferior to 0.5) for both patients (i.e., patients e and f).

### Example 2

### Validation of Candidate Biomarkers

Immunoassay experiments were carried out to confirm the quantitative measurements obtained with MS/MS. The choice of candidate biomarkers to be assessed was essentially based on the availability of commercial and in-house developed immunoassays.

GSTP1 protein (MW=23 kDa) was probed with immunoblot analysis in pooled microdialysates samples (n=3) as illustrated in FIG. 2. The increase in the IC microdialysate with respect to the CT was undisputable, and corroborated the TMT-based discovery results as well as previous studies of post- and ante-mortem CSF (15, 20).

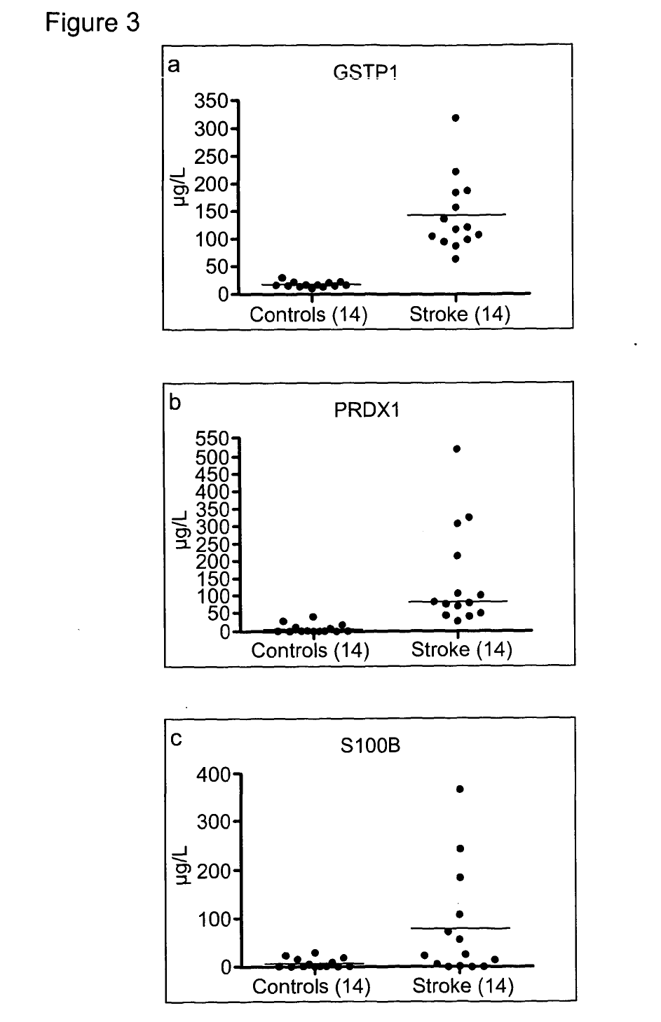

Second, ELISAs were performed for GSTP1, PRDX1, and S100B on sera of control and stroke patients (n=28). The ELISA results are given in FIG. 3 and summarized in Table 13.

TABLE 13
Result summary for GSTP1, PRDX1, and S100B levels
in sera samples quantified with ELISA.

| | Control (n = 14) | | Stroke (n = 14) | | p^{ç} |
|---|---|---|---|---|---|
| GSTP1 (μg · L⁻¹) | | | | | |
| Mean ± standard deviation | 16.9 ± 5.1 | | 143.1 ± 67.5 | | 0.0002 |
| Median (minimum-maximum) | 15.7 | (8.6-29.0) | 119.0 | (63.5-320.0) | |
| PRDX1 (ng · L⁻¹) | | | | | |
| Mean ± standard deviation | 7.3 ± 12.7 | | 145.7 ± 144.0 | | 0.0001 |
| Median (min-max) | 0.0 | (0.0-41.2) | 81.9 | (26.7-519.5) | |
| S100B (μg · L⁻¹) | | | | | |
| Mean ± standard deviation | 7.5 ± 10.0 | | 78.9 ± 113.1 | | 0.0093 |
| Median (minimum-maximum) | 1.8 | (0.0-28.7) | 23.1 | (0.0-369.2) | |

^{ç}Wilcoxon matched pairs test.

GSTP1 was found significantly elevated in the blood of stroke patients compared to controls (p=0.0002, Wilcoxon matched pairs test). The mean ratio in blood between stroke patients and controls was 8.47 (Table 13); i.e., three-times more than the ratio IC/P found in brain microdialysis samples (Expa, Table 7). Among the peroxiredoxin family, blood PRDX1 enabled to differentiate control from stroke patients at the p=0.0001 level of significance. An increase of its levels of almost 20-times was observed in the stroke population. In accordance with results previously described in the literature (28, 29), the concentration measurements of blood S100B were significantly higher in stroke patients than controls (p=0.0093).

Thus we disclose protein markers of stroke which we have illustrated by comparisons of microdialysis samples from the IC, P, and CT of ischemic stroke patients. Human brain microdialysates were analysed using an isobaric tagging technology coupled to peptide isoelectric focusing fractionation, and RP-LC MS/MS analysis. Increased levels of GSTP1, PRDX1 and S100B in the IC microdialysates were further verified with immunoblot on pooled microdialysis samples and/or ELISA on blood of control and stroke patients. Thus we have clearly established the utility and applicability of the markers and methods presented herein.

### Example 3

### Protein Amounts in Microdialysis Samples

Analyses with 1-D PAGE of the different microdialysis samples under study revealed slightly different patterns as well as large variations in the total concentration of proteins between samples (FIG. 1 and FIG. 5). These variations may possibly result from recovery issues for instance through the microdialysis membrane in addition to the biological variations and/or the severity of stroke. In a previous study of CT microdialysate of stroke patients, the protein concentration in 18 samples was determined to range from 0.083 to 0.395 g·L−1 with a mean content of 0.21±0.11 g·L−1 (11). Smaller molecules such as glutamate, glycerol, lactate, and pyruvate were measured in 50 patients over the same sampling period respectively at 3.9±0.3 μmol·L−1, 38.9±1.9 μmol·L−1, 2.0±0.1 mmol·L−1, and 58.4±3.3 μmol·L−1 in the CT microdialysates, whereas in the IC the median for the same molecules was respectively 196.5 μmol·L−1 (ranging from 126.0 to 453.0), 600.5 μmol·L−1 (ranging from 464.2 to 1187.1), 6.1 mmol·L−1 (ranging from 0.1 to 12.0), and 17.4 μmol·L−1 (ranging from 4.2 to 591.7) (30). In brief, the protein concentration varied considerably from one sample to another in the CT microdialysates, whereas the concentrations of small molecules were uniform in the CT but more heterogeneous in the IC. Thus, a large protein concentration variation might be expected in the IC samples, as well as in the P microdialysates. Protein concentration variations were confirmed here.

Because of the differences in the total protein concentration, equalization of the samples was needed to carry out the quantitative proteomic study.

The samples to compare were equalized according to their protein amount (i.e., weight) before the quantitative analysis.

1-D PAGE images were used to compare the sample concentrations with densitometry (see Experimental Procedures). According to this relative protein quantitation, equal protein amounts between pairs to compare were taken for TMT2-based quantitative assessment.

As a consequence, a further normalization was performed on the TMT2 quantitative data. We hypothesized that most proteins, and therefore most peptides and reporter-ion signals, should be equal among samples (shown in FIG. 4). Accordingly, the common areas between the frequency distribution of the peptides relative abundance of both TMT2-labeled pairs had to be maximal. Besides, this processing was coherent with the first normalization performed from 1-D PAGE images.

### Example 4

### Increased and Decreased Proteins

To the best of our knowledge, this is the most-extensive proteomic study of human brain microdialysates, and the first one targeting brain ischemia (6). Through the study we have obtained a quantitative map of human brain microdialysates, as a monitoring of ECF in the brain of stroke patients. Depending on the brain region probed with microdialysis, relevant protein markers of stroke were discovered.

Many of the found proteins were identified previously in CSF (13, 20, 31). More precisely, several proteins with increased amount within the compared pairs (Tables 7-9) were previously identified in a comparative study of ante- and post-mortem CSF (13). This was the case for instance for cystatin-B, GFAP, S100B, PRDX1, and peroxiredoxin-2 (PRDX2). In that previous study, PRDX1 was increased with a ratio of 14.74 in post-mortem CSF compared to ante-mortem CSF. The correlation of many of the quantitative results between both studies not only validated the post-mortem CSF as a model of massive brain injury, but also highlighted the value of the quantitative proteome map obtained with the microdialysis samples.

In Tables 7-9, some proteins exhibited increased and decreased amounts. This was the case for fibrinogen alpha chain (FIBA), platelet basic protein, profilin-1, carbonic anhydrase 1, and GFAP. With a molecular weight of 95 kDa, FIBA might have been recovered inefficiently through the dialysis membrane. The variations could not be directly explained for the other proteins, yet, for instance, GFAP (50 kDa) can dimerize and oligomerize, as well as co-polymerize with other protein like vimentin, desmin, and annexin (32). Its recovery might have then been altered. When the sample in the methods of the invention is other than microdialysate, e.g. when the sample is CSF or blood, such problem(s) are advantageously avoided since there is no molecular weight cut-off when using such samples.

Ischemic stroke is caused by the disturbance of blood flow supplied to the brain. Cerebral blood flow was shown to be decreased in penumbra, and even more in infarct core (33). Interestingly, most of the decreased proteins found in the IC vs. P, IC vs. CT, and P vs. CT studies (Tables 10-12) were blood proteins (e.g., serum albumin, serotransferrin, haptoglobin, hemoglobins), somehow reflecting the regional variation of altered blood flow in these distinct brain areas.

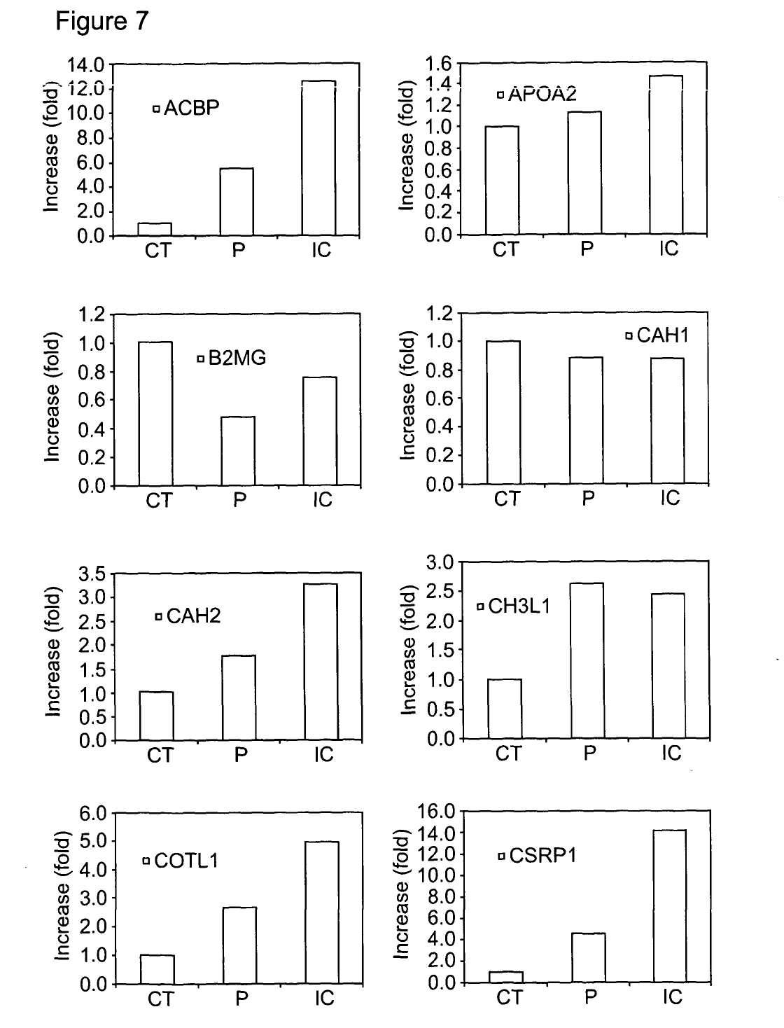

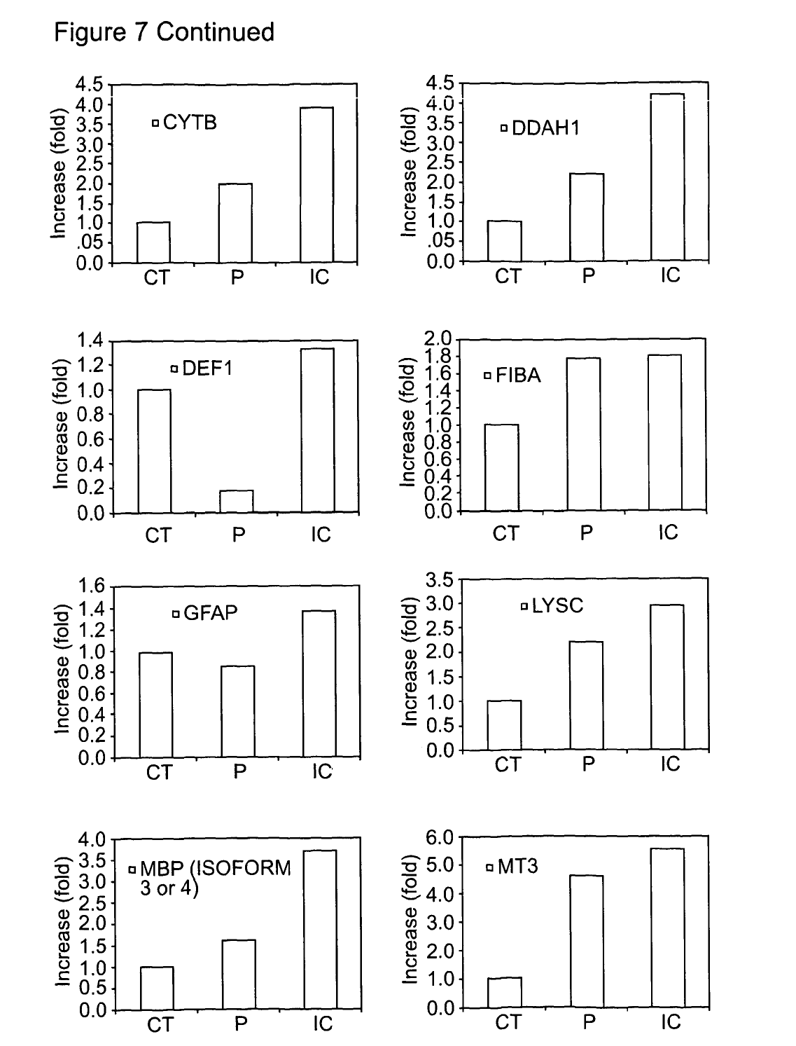

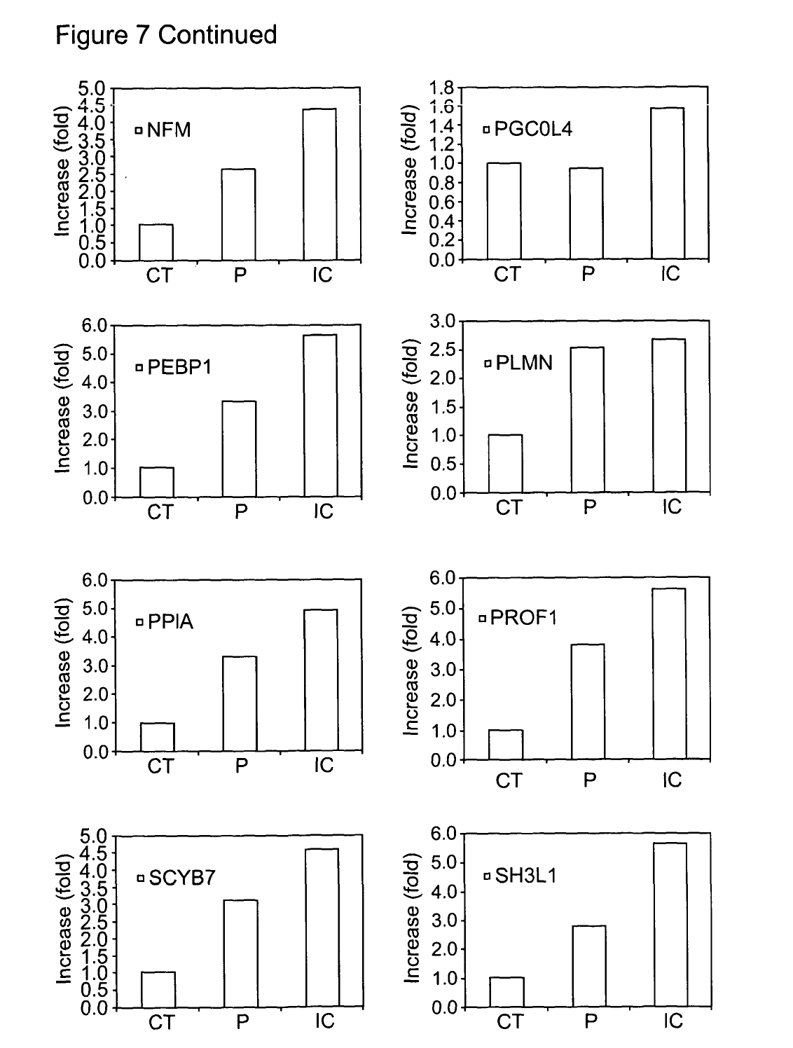

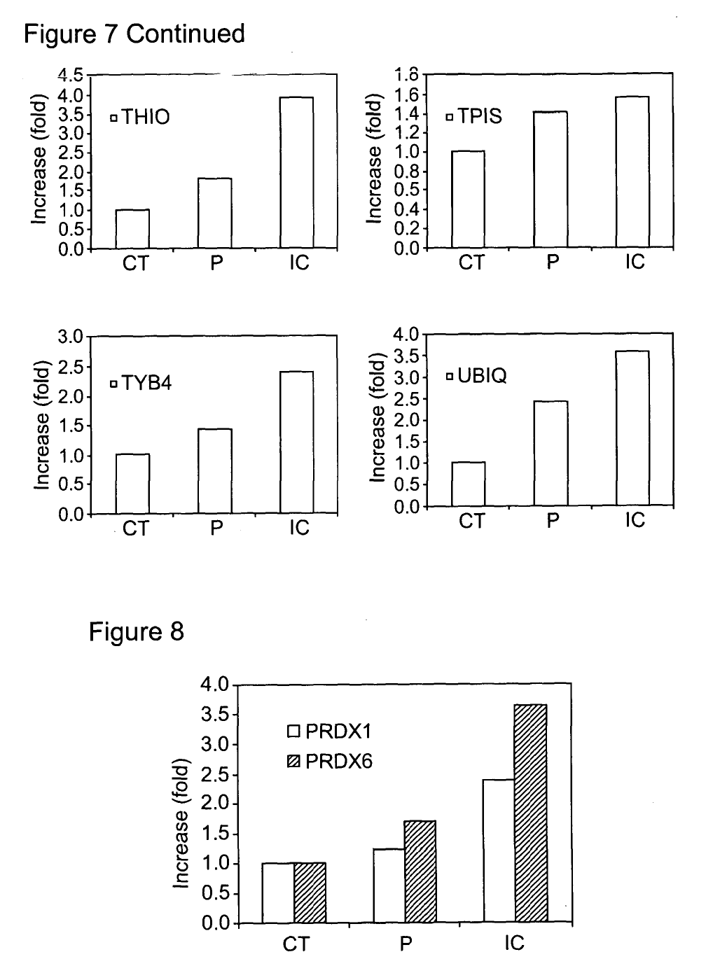

FIG. 7 displays the evolution of the protein levels that was observed in the MDs for proteins reported in the Tables above. Most of these proteins were found to increase from CT to P and from P to IC MDs. As shown in FIG. 8, PRDX1 and PRDX6 were found to increase from CT, P, and IC MDs. In most of the cases, the progression/elevation of the protein levels from the CT to the IC, as displayed in FIGS. 7 and 8, reflects a direct relationship with the severity of the cerebral damage. These results show a relevant biological trend to reinforce the medically relevant aspects of the invention.

### Example 5

### Further Validation of Biomarkers

Several identified proteins were selected to demonstrate the validity of our discovery approach, based on the availability of an alternative diagnostic tool (i.e., ELISA) and/or a strong scientific rationale for involvement in brain ischemia. S100B, a well-documented biomarker of brain damage (34), is a calcium binding and growth-regulating secretory protein that is highly expressed in brain tissues (9). The concentration of S100B has been assessed in many brain insults and dysfunctions. S100B was increased in stroke (28, 29), SAH (35), and TBI (36). S100B was previously measured in the brain ECF of two patients with acute brain injury using the microdialysis technique (37). The detection and increased level determination of S100B in one IC microdialysate compared to a P sample, as well as its validation in the blood of stroke patients, confirmed the findings reported here, and demonstrated the great value of the studied samples.

GSTP1 protein is an enzyme that is able to inactivate many toxic, electrophiles and organic peroxides (38). GSTP1 is one the three glutathione S-transferases described in the central nervous system (39). Several studies suggested its association with Parkinson's disease (40). High levels of GSTP1 were recently reported in CSF of late stage patients suffering human African trypaniosomiasis (21). The protein is known to be associated with early brain cell death because it was found with increased concentration in CSF of deceased patient compared to alive ones (20). High correlation of the increase of GSTP1 in microdialysis and blood samples stressed the relevance of the obtained quantitative proteome maps of the brain microdialysates of stroke patients as a pertinent model for the discovery of brain markers.

Peroxiredoxins are ubiquitous antioxidant enzymes involved in the degradation of oxygen peroxide and other reactive oxygen species (41, 42). These thiol-specific antioxidant proteins are also termed thioredoxin peroxidases. The family of peroxiredoxins is composed of six distinct groups that can be classified in two categories, the 1-Cys and 2-Cys peroxiredoxins, according to the number of cysteine residues involved in the reduction process. Peroxiredoxin-6 (PRDX6) is actually the sole 1-Cys member. In the brain, PRDX1 and PRDX6 were shown to be primarily expressed in astrocytes whereas PRDX2 was expressed exclusively in neurons (43, 44). PRDX2 was significantly increased in the substantia nigra from Parkinson's disease patients (45), and in the frontal cortex and cerebellum of patients with Down syndrome, Alzheimer's disease, and Pick's disease (46). PRDX1 was demonstrated to be part of an adaptive response to oxidative stress in brain endothelial cells and have protective effects at the injured blood-brain barrier (47). Herein, the increased amounts and increased concentrations of PRDX1 in respectively the microdialysates of the injured parts of the brain, and the blood of stroke patients appeared therefore highly relevant for further investigation in cerebrovascular diseases. Very interestingly, PRDX1 and GSTP1 are implicated in similar redox protective mechanisms, and were evidenced to interact together (48). As well, GSTP1 was shown to reactivate oxidized PRDX6 (49) through the formation of a complex (50).

Malignant MCA infarction patients as those included in our study are severely impaired patients that receive several treatments at the neurointensive care units, such as moderate hypothermia, that might modify the expression pattern of some of the described proteins. Another limitation is that the recovery rates through the 100 kDa microdialysis probes are unknown for most of the discovered proteins. It may advantageously be possible to alleviate these limitations by choosing a sample which is not collected through a molecular weight-limited route e.g. by using CSF or blood as the sample.

In conclusion, the present study explored the brain microdialysates of stroke patients through proteomic analysis. Qualitative results offered an extensive proteome map of microdialysates, and extracellular fluid from the human brain. Moreover, quantitative comparisons of microdialysates of several areas of the human ischemic brain were shown to provide a valuable source of biomarkers for cerebrovascular diseases. Several of the increased proteins were verified on blood samples of a small cohort of control and stroke patients. The correlation between discovery and early validation data demonstrated that many of the discovered proteins represent biomarkers for the diagnosis and/or prognosis of stroke, as well as other acute brain damage related disorders.

### Example 6

### Changes in Protein Levels Associated with Decreased Cerebral Blood Flow

In vivo human brain extracellular fluids (ECF) of acute ischemic stroke patients were investigated to assess the changes in protein levels associated to decreased cerebral blood flow. Microdialysates (MDs) from the infarct core (IC), the penumbra (P), and the unaffected contralateral (CT) brain regions of patients suffering an ischemic stroke were compared using a shotgun proteomic approach based on isobaric tagging and mass spectrometry (MS). Quantitative analysis showed 53 proteins with increased amounts in the IC or P with respect to the CT samples. Glutathione S-transferase P (GSTP1), peroxiredoxin-1 (PRDX1), and protein S100-B (S100B) were further assessed with ELISA on the blood of unrelated control and stroke patients (n=28). Significant increases of 8, 20, and 11-fold were found respectively. Taken together, these results demonstrated clear differences in ECF protein levels between P and IC associated to ischemic damages. In addition, the evaluation of PRDX1 highlighted the value of ECF as an efficient source to further discover blood stroke markers.

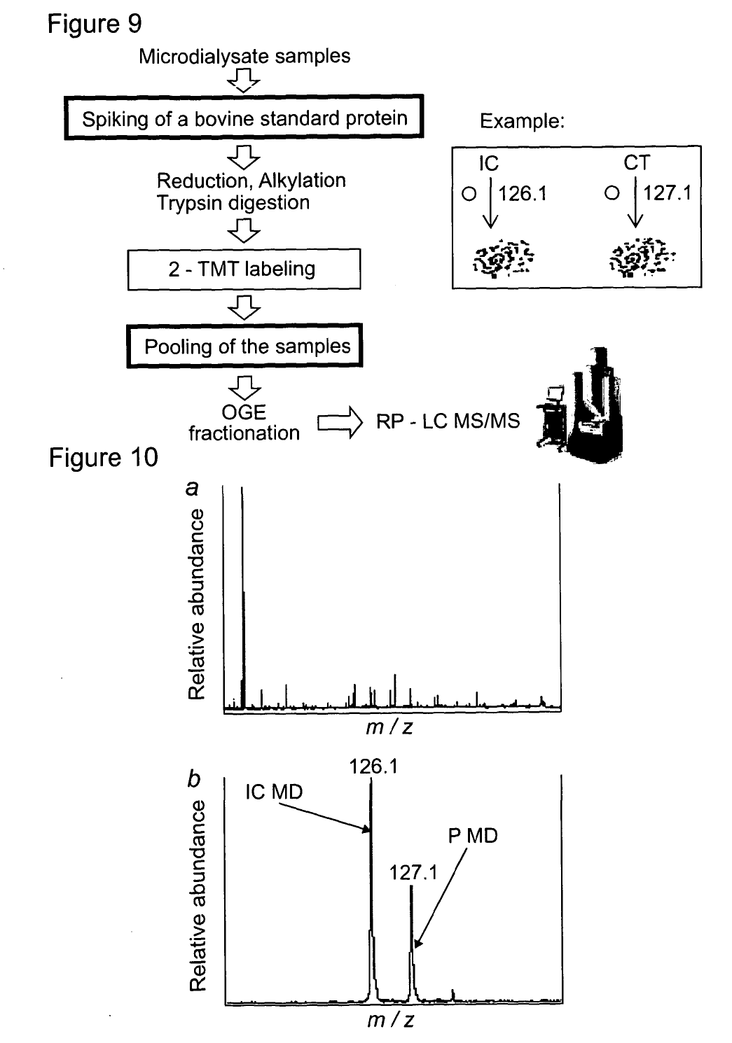

Microdialysis sampling of stroke patients was approved by the local institutional ethical committee. Malignant middle cerebral artery infraction patients were monitored with high-cut-off (100 kDa) cerebral microdialysis catheters. Computed tomography scan was used to confirm brain microdialysis catheter location. MDs were obtained hourly for 5 days after perfusion with an artificial CSF solution. Proteomic analysis was performed on brain MDs obtained during the first 24 h of brain monitoring. The 2-plex isobaric Tandem Mass Tag (TMT) technology (Dayon et al 2008) was used to label trypsin-digested extracts from two brain regions of six patients suffering stroke (FIG. 9). Following the labeling, the pooled samples were first fractionated by off-gel electrophoresis (OGE). The fractions were then analyzed by reversed-phase liquid chromatography (RP-LC) and matrix-assisted laser desorption tandem time-of-flight (MALDI TOF/TOF) MS (FIG. 10). The identification and quantitation of proteins were assessed with stringent criteria using Phenyx search in the Swiss-Prot human database.

Immunoblot validation was carried out for GSTP1. Pooled IC and CT MDs (n=3) were separated with 1-D SDS PAGE (15%). Immunodetection was performed with the anti-human GSTP1 rabbit polyclonal antibody. S100B, GSTP1, and PRDX1 were further validated with ELISA of blood of control and stroke patients (n=28). S100B and PRDX1 were validated using commercial ELISA kits. Concerning GSTP1, no commercial assay being currently available, a sandwich home-made was developed as previously described in (Burgess et al 2006; Hainard et al 2009).

Microdialysis is a bioanalytical sampling tool to continuously monitor events occurring in living tissues. It is based on probing ECF and allows collecting endogenous substances from the extracellular space, which can diffuse through the semi-permeable membrane at the tip of the microdialysis probe. Such a technique is quite appropriate to search and follow biochemical markers in real-time in many organs. The proteomic comparisons of human brain MDs showed significantly over-represented proteins (with a ratio superior to 2) in the IC compared to the CT and P counterparts (FIG. 10). Proteins such as glial fibrillary acidic protein (GFAP) and S100B have already been reported to be associated to brain damage and appeared to be increased in one or several IC MDs. FIG. 8 displays an example of the evolution of the protein levels that was observed in the MDs for 2 peroxiredoxin proteins. Many proteins were found to increase from CT to P and from P to IC MDs.

Similarly, the increase in GSTP1 was validated with immunoblot experiments in pooled MDs (n=3) as illustrated in FIG. 2.

The level of S100B, GSTP1, and PRDX1 in serum was also measured by ELISA in the serum of 14 stroke patients and 14 controls (FIG. 3) demonstrating their utility as peripheral markers of brain damage caused by reduced blood flow in ischaemic stroke.

The GSTP1 concentration was found significantly elevated in the blood of stroke patients compared to controls (p=0.0002, Wilcoxon matched pairs test). The mean ratio in blood between stroke patients and controls was 8.47. Blood PRDX1 level enabled to differentiate control from stroke patients at the p=0.0001 level of significance. An increase of its levels of almost 20-times was observed in the stroke population. In accordance with previous results (Buttner et al 1997; Missler et al 1997), the concentration measurements of blood S100B were significantly higher in stroke patients than controls (p=0.0093).

This example explored the brain MDs of stroke patients with proteomic analysis. Qualitative results offered an extensive proteome map of microdialysates and ECF from the human brain. Moreover, quantitative comparisons of MDs of the IC, P and CT parts of the human brain were shown to provide a valuable source of biomarkers for cerebrovascular diseases. Several of the increased proteins were verified on a small cohort of control and stroke patients. The correlation between discovery and early validation data demonstrated the industrial application of the invention for the diagnosis and/or prognosis of stroke, as well as other brain damage related disorders.

### Example 7

### Selection of Enlarged Panel Proteins

In vivo human brain extracellular fluids (ECF) of acute ischemic stroke patients were previously investigated to assess the changes in protein levels associated to decreased cerebral blood flow as described herein. Microdialysates (MDs) from the infarct core (IC), the penumbra (P), and the unaffected contralateral (CT) brain regions of patients suffering an ischemic stroke (n=6) were compared using a shotgun proteomic approach based on isobaric tagging and mass spectrometry (MS). Quantitative analysis showed 53 proteins with increased amounts in the IC or P with respect to the CT samples. These results demonstrated clear differences in ECF protein levels between CT, P and IC associated to ischemic damage. Glutathione S-transferase P (GSTP1), peroxiredoxin-1 (PRDX1), and protein S100-B (S100B) were further assessed with ELISA on the blood of unrelated control (n=14) and stroke (n=14) patients. Significant increases of 8 (p=0.0002), 20 (p=0.0001), and 11-fold (p=0.0093) were found respectively. These highlighted the value of ECF as an efficient source to further discover blood stroke markers.

Whilst GSTP-1 and Peroxiredoxins 1 and 6 represent useful markers for management of stroke, we wished to construct larger panels of proteins to further improve diagnostic sensitivity and/or specificity and/or provide prognostic information. We therefore undertook the verification and validation of the stroke biomarker candidates found previously in MDs.

Following a comprehensive bioinformatic analysis of candidate proteins, three groups of biomarkers were selected in descending priority order:

| PANEL A | ID | Description |
|---|---|---|
| N^{o}1 | ACBP_HUMAN | Acyl-CoA-binding protein |
| N^{o}2 | CSRP1_HUMAN | Cysteine and glycine-rich protein 1 |
| N^{o}3 | PEBP1_HUMAN | Phosphatidylethanolamine-binding |
| | | protein 1 |
| N^{o}4 | DDAH1_HUMAN | N(G),N(G)-dimethylarginine |
| | | dimethylaminohydrolase 1 |
| N^{o}5 | MT3_HUMAN | Metallothionein-3 (MT-3) |
| N^{o}6 | CYTB_HUMAN | Cystatin-B |

| PANEL B | ID | Description |
|---|---|---|
| N^{o}1 | PPIA_HUMAN | Peptidyl-prolyl cis-trans isomerase A |
| N^{o}2 | NFM_HUMAN | Neurofilament medium polypeptide |
| N^{o}3 | UBIQ_HUMAN | Ubiquitin. |
| N^{o}4 | B2MG_HUMAN | Beta-2-microglobulin precursor |
| N^{o}5 | CYTC_HUMAN | Cystatin-C precursor (Cystatin-3) |
| N^{o}6 | SH3L1_HUMAN | SH3 domain-binding glutamic acid-rich- |
| | | like protein. |
| N^{o}7 | TPIS_HUMAN | Triosephosphate isomerase |
| N^{o}8 | MBP_HUMAN | Myelin basic protein (MBP) |
| N^{o}9 | MT2_HUMAN | Metallothionein-2 (MT-2) |

| PANEL C | ID | Description |
|---|---|---|
| N^{o}1 | NFM_HUMAN | Neurofilament medium polypeptide |
| N^{o}2 | COTL1_HUMAN | Coactosin-like protein. |
| N^{o}3 | THY1_HUMAN | Thy-1 membrane glycoprotein precursor |
| N^{o}4 | PROF1_HUMAN | Profilin-1 |
| N^{o}5 | TYB4_HUMAN | Thymosin beta-4 |
| N^{o}6 | MT1E_HUMAN | Metallothionein-1E |
| N^{o}7 | FABPB_HUMAN | Fatty acid-binding protein, brain (B-FABP) |
| N^{o}8 | GFAP_HUMAN | Glial fibrillary acidic protein (GFAP). |
| N^{o}9 | CAH2_HUMAN | Carbonic anhydrase 2 |
| N^{o}10 | CERU_HUMAN | Ceruloplasmin precursor |
| N^{o}11 | DCD_HUMAN | Dermcidin precursor |
| N^{o}12 | DEF1_HUMAN | Neutrophil defensin 1 precursor (HNP-1 |

Together, Panels A, B and C form an enlarged panel, referred to as enlarged panel ABC.

Among the 53 biomarker candidates reported above, N(G);N(G)-dimethylarginine dimethylaminohydrolase 1 (DDAH1_HUMAN), cystatin-B (CYTB_HUMAN), acyl-CoA-binding protein (ACBP_HUMAN), cysteine and glycine-rich protein 1 (CSRP1_HUMAN), metallothionein-3 (MT3_HUMAN), and phosphatidylethanolamine-binding protein 1 (PEPB1_HUMAN) (Panel A) have been prioritised.

### Example 8

### Selected Reaction Monitoring Mass Spectrometry Method for Measuring Signature-Peptides of Stroke Biomarker Candidates

To provide further validation of the enlarged panel proteins single protein and multiplex protein assays are developed using immunoassay (ELISA) and mass spectrometry (MRM) methods.

This example demonstrates the rapid ability of MRM to develop a multiplex panel. In this example we selected proteins from Panel A of example 7 to use in order to illustrate the method. However, it is not intended that this method be limited to that specific panel of biomarkers. This panel of biomarkers is being used as a convenient panel to help understand how to carry out one advantageous mode of detection. The same mode of detection can be used for any other group of markers disclosed herein, simply by following the method set out here but instead using the markers of a different panel or group as disclosed.

Thus, this example shows the development and evaluation of a method based on selected reaction monitoring (SRM) MS to detect selectively signature-peptides of the prioritised stroke biomarker candidates of Panel A.

### Design of the Method

Design of an MRM method first requires selection of target peptides representative of each marker protein (proteotypic peptides). The second step involves selection of specific peptide fragments that will arise in collision-induced dissociation of the parent peptide during tandem mass spectrometry. The difference in the mass-to-charge (m/z) ratio of the parent and daughter ions are known as transitions.

An in silico approach was used to select proteotypic tryptic signature-peptides representative of each stroke biomarker candidate. A total of 7, 4, 7, 3, 3 and 6 proteotypic signature-peptides were selected for DDAH1, CYTB, ACBP (3 isoforms), CSRP1, MT3 and PEPB1 respectively.

The signature-peptide selection was based on i) uniqueness of the peptide sequence in the human protein database (UniProt Swiss-Prot) determined with the home-made Proteotype software, ii) m/z value of the peptide precursor-ion for relevant MS detection, and iii) absence of cysteine and methionine residues in the sequence when possible (Table 14 below).

TABLE 14
Proteotypic peptides of proteins useful in the diagnosis and/or prognostic
monitoring of a subject with acute brain damage

| | | | | | observation in |
|---|---|---|---|---|---|
| | | | | | the Peptide |
| | | | | | Atlas |
| | | | | | (http://www. |
| | | | | | peptideatlas. |
| | | | | | org/accessed on |
| accession | description | prev.res | sequence | next.res | May 04, 2011) |
| sp\|O94760\| | N(G),N(G)- | R | [symbol] | S | 3 |
| DDAHl_HUMAN | dimethylarginine | K | [symbol] | E | 6 |
| | dimethylaminohydrolase 1 | K | [symbol] | S | 55 |
| | | R | [symbol] | D | 4 |
| | | K | [symbol] | A | 0 |
| | | R | [symbol] | L | 0 |
| | | R | [symbol] | V | 4 |
| sp\|P04080\| | Cystatin-B | K | [symbol] | H | 0 |
| CYTB_HUMAN | | K | [symbol] | V | 56 |
| | | R | [symbol] | A | 367 |
| | | K | VHVGDEDFVHLR | V | 564 |
| sp\|P07108\| | Acyl-CoA-binding | — | MSQAEFEK | A | 0 |
| ACBP_HUMAN | protein | K | AAEEVR | H | 0 |
| | | K | QATVGDINTERPGMLDFTGK | A | 342 |
| | | K | TKPSDEEMLFIYGHYK | Q | 63 |
| | | K | [symbol] | G | 12 |
| sp\|P07108-2\| | Isoform 2 of Acyl- | — | MWGDLWLLPPASANPGTGTEA | A | 0 |
| ACBP_HUMAN | CoA-binding protein | | EFEK | | |
| sp\|P07108-3\| | Isoform 3 of Acyl- | — | MPAFAEFEK | A | 0 |
| ACBP_HUMAN | CoA-binding protein | | | | |
| sp\|P21291\| | Cysteine and | K | [symbol] | G | 30 |
| CSRP1_HUMAN | glycine-rich | K | [symbol] | D | 2 |
| | protein 1 | K | [symbol] | G | 4 |
| sp\|P25713\| | Metallothionein-3 | K | [symbol] | C | 0 |
| MT3_HUMAN | | — | MDPETCPCPSGGSCTCADSCK | C | 0 |
| | | K | SCCSCCPAECEK | C | 0 |
| sp\|P30086\| | Phosphatidylethanolamine- | K | [symbol] | G | 412 |
| PEBP1_HUMAN | binding protein 1 | K | [symbol] | — | 10 |
| | | K | [symbol] | K | 159 |
| | | K | [symbol] | L | 467 |
| | | K | [symbol] | N | 319 |
| | | R | [symbol] | C | 47 |
| sP\|P09211\| | Glutathione | K | TFIVGDQISFADYNLLDLLLI | L | ND |
| GSTP1_HUMAN | S-transferase P | | HEVLAPGCLDAFPLLSAYVGR | | |
| | | — | MPPYTVVYFPVR | G | ND |
| | | K | DDYVK | A | ND |
| | | K | DQQEAALVDMVNDGVEDLR | C | ND |
| | | K | FQDGDLTLYQSNTILR | H | ND |
| | | K | ASCLYGQLPK | F | ND |
| | | K | AFLASPEYVNLPINGNGK | Q | ND |
| | | R | MLLADQGQSWK | E | ND |
| | | R | LSARPK | L | ND |
| | | R | TLGLYGK | D | ND |
| | | K | EEVVTVETWQEGSLK | A | ND |
| | | K | ALPGQLKPFETLLSQNQGGK | T | ND |
| | | K | YISLIYTNYEAGK | D | ND |
| sp\|P30041\| | Peroxiredoxin-6 | K | GMPVTAR | V | ND |
| PRDX6_HUMAN | | — | MPGGLLLGDVAPNFEANTTVGR | I | ND |
| | | R | DFTPVCTTELGR | A | ND |
| | | R | VVFVFGPDK | K | ND |
| | | K | LIALSIDSVEDHLAWSK | D | ND |
| | | R | ELAILLGMLDPAEK | D | ND |
| | | K | LSILYPATTGR | N | ND |
| | | R | VATPVDWK | D | ND |
| | | R | NFDEILR | V | ND |
| | | K | LPFPIIDDR | N | ND |
| | | R | VVISLQLTAEK | R | ND |
| | | K | DINAYNCEEPTEK | L | ND |
| | | K | LAPEFAK | R | ND |
| | | K | DGDSVMVLPTIPEEEAK | K | ND |
| | | R | FHDFLGDSWGILFSHPR | D | ND |
| sp\|Q06830\| | Peroxiredoxin-1 | K | HGEVCPAGWKPGSDTIKPDVQK | S | ND |
| PRDX1_HUMAN | | K | QGGLGPMNIPLVSDPK | R | ND |
| | | K | ADEGISFR | G | ND |
| | | K | DISLSDYK | G | ND |
| | | R | LVQAFQFTDK | H | ND |
| | | K | IGHPAPNFK | A | ND |
| | | K | LNCQVIGASVDSHFCHLAWVNT | K | ND |
| | | | PK | | |
| | | K | YVVFFFYPLDFTFVCPTEIIAF | A | ND |
| | | | SDR | | |
| | | — | MSSGNAK | I | ND |
| | | R | TIAQDYGVLK | A | ND |
| | | K | ATAVMPDGQFK | D | ND |

| | observation | | | | | | |
|---|---|---|---|---|---|---|---|
| | in discovery | | frequency | | | | |
| | (J. Proteome | | in human_ | | | | |
| | Res., 2011, | | uniprot_ | | | | |
| | 10(3), pp | | sprot_ | | predicted | | rt |
| accession | 1043-1051) | position | varsplic | mass | charge | m/z | factor |
| sp\|O94760\| | 3 | 20:31 | 1 | 1291.712 | 3 | 431.2 | 42.3 |
| DDAHl_HUMAN | 0 | 121:136 | 1 | 1679.787 | 2 | 840.4 | 55.2 |
| | 0 | 160:175 | 1 | 1684.89 | 3 | 562.3 | 56.2 |
| | 2 | 150:159 | 1 | 1064.562 | 2 | 532.8 | 56.1 |
| | 0 | 35:42:00 | 1 | 874.4265 | 2 | 437.7 | 26.6 |
| | 0 | 46:57:00 | 1 | 1328.732 | 3 | 443.6 | 42.9 |
| | 2 | 238:247 | 1 | 1150.526 | 2 | 575.8 | 17.8 |
| sp\|P04080\| | 0 | 92:98 | 1 | 924.4098 | 2 | 462.7 | 49.6 |
| CYTB_HUMAN | 0 | 45:56:00 | 1 | 1326.705 | 2 | 663.9 | 51.7 |
| | 0 | 69:89 | 1 | 2458.272 | 4 | 615.3 | 47.1 |
| | 4 | 57:68 | 1 | 1422.712 | 4 | 356.4 | 38.4 |
| sp\|P07108\| | 0 | 01:08 | 1 | 969.4346 | 2 | 485.2 | 23.3 |
| ACBP_HUMAN | 1 | 09:14 | 3 | 674.3468 | 2 | 337.7 | 19.0 |
| | 0 | 34:53:00 | 3 | 2150.055 | 3 | 717.4 | 55.0 |
| | 6 (3 × | 18:33 | 3 | 1957.936 | 4 | 490.2 | 58.1 |
| | oxidised) | | | | | | |
| | 7 | 56:63 | 3 | 1061.505 | 2 | 531.3 | 41.1 |
| sp\|P07108-2\| | 0 | 7108:7132 | 1 | 2717.292 | 2 | 1359 | 88.9 |
| ACBP_HUMAN | | | | | | | |
| sp\|P07108-3\| | 0 | 7108:7116 | 1 | 1069.502 | 2 | 535.3 | 38.9 |
| ACBP_HUMAN | | | | | | | |
| sp\|P21291\| | 3 | 179:193 | 1 | 1433.681 | 2 | 717.3 | 59.2 |
| CSRP1_HUMAN | 0 | 152:161 | 1 | 1034.536 | 2 | 517.8 | 41.9 |
| | 0 | 70:84 | 1 | 1474.681 | 2 | 737.8 | 31.1 |
| sp\|P25713\| | 8 | 53:63 | 1 | 1061.475 | 2 | 531.2 | 20.0 |
| MT3_HUMAN | 0 | 01:21 | 1 | 2376.833 | 2 | 1189 | −5.4 |
| | 6 | 33:44:00 | 1 | 1547.505 | 2 | 774.3 | 3.5 |
| sp\|P30086\| | 7 | 94:113 | 1 | 1949.945 | 2 | 975.5 | 45.9 |
| PEBP1_HUMAN | 11 | 180:187 | 1 | 937.4989 | 2 | 469.3 | 27.8 |
| | 10 | 63:76 | 1 | 1560.827 | 2 | 780.9 | 48.1 |
| | 5 | 48:62 | 1 | 1632.798 | 3 | 544.9 | 36.7 |
| | 1 | 40:47:00 | 1 | 885.5404 | 2 | 443.3 | 29.4 |
| | 6 | 120:132 | 1 | 1708.906 | 3 | 570.3 | 59.1 |
| sP\|P09211\| | ND | 142:183 | 1 | 4649.436 | 3 | 1550 | 223.3 |
| GSTP1_HUMAN | ND | 01:12 | 1 | 1468.766 | 2 | 734.9 | 44.04 |
| | ND | 117:121 | 1 | 639.2984 | 2 | 320.2 | 24.38 |
| | ND | 83:101 | 1 | 2116.982 | 2 | 1059 | 70.67 |
| | ND | 56:71 | 1 | 1883.95 | 2 | 942.5 | 62.89 |
| | ND | 46:55:00 | 1 | 1136.577 | 2 | 568.8 | 37.34 |
| | ND | 192:209 | 1 | 1903.991 | 2 | 952.5 | 65.59 |
| | ND | 20:30 | 1 | 1276.635 | 2 | 638.8 | 47.63 |
| | ND | 184:189 | 1 | 671.4199 | 3 | 224.5 | 1.119 |
| | ND | 76:82 | 1 | 751.4349 | 2 | 376.2 | 39.09 |
| | ND | 31:45:00 | 1 | 1733.859 | 2 | 867.4 | 58.34 |
| | ND | 122:141 | 1 | 2126.16 | 3 | 709.4 | 60.47 |
| | ND | 104:116 | 1 | 1534.779 | 2 | 767.9 | 55.73 |
| sp\|P30041\| | ND | 126:132 | 1 | 731.3869 | 2 | 366.2 | 30.53 |
| PRDX6_HUMAN | ND | 01:22 | 1 | 2229.133 | 2 | 1115 | 63.65 |
| | ND | 42:53:00 | 1 | 1395.657 | 2 | 698.3 | 40.89 |
| | ND | 133:141 | 1 | 1007.556 | 2 | 504.3 | 49.98 |
| | ND | 68:84 | 1 | 1897.007 | 3 | 633 | 79.81 |
| | ND | 109:122 | 1 | 1512.834 | 2 | 756.9 | 91.41 |
| | ND | 145:155 | 1 | 1191.673 | 2 | 596.3 | 37.97 |
| | ND | 175:182 | 1 | 915.4934 | 2 | 458.3 | 34.03 |
| | ND | 156:162 | 1 | 906.4679 | 2 | 453.7 | 49.47 |
| | ND | 98:106 | 1 | 1085.599 | 2 | 543.3 | 47.47 |
| | ND | 163:173 | 1 | 1200.72 | 2 | 600.9 | 56.15 |
| | ND | 85:97 | 1 | 1582.669 | 2 | 791.8 | 26.56 |
| | ND | 57:63 | 1 | 775.4348 | 2 | 388.2 | 28.4 |
| | ND | 183:199 | 1 | 1829.884 | 2 | 915.4 | 63.4 |
| | ND | 25:41:00 | 1 | 2030.987 | 4 | 508.5 | 76.44 |
| sp\|Q06830\| | ND | 169:190 | 1 | 2406.187 | 5 | 482 | 22 |
| PRDX1_HUMAN | ND | 94:109 | 1 | 1622.857 | 2 | 811.9 | 58.44 |
| | ND | 121:128 | 1 | 894.4316 | 2 | 447.7 | 35.7 |
| | ND | 28:35:00 | 1 | 940.4622 | 2 | 470.7 | 42.1 |
| | ND | 159:168 | 1 | 1196.631 | 2 | 598.8 | 47.24 |
| | ND | 08:16 | 1 | 980.5312 | 3 | 327.5 | 16.05 |
| | ND | 69:92 | 1 | 2753.329 | 4 | 689.1 | 63.37 |
| | ND | 38:62 | 1 | 3093.511 | 2 | 1547 | 153.1 |
| | ND | 01:07 | 1 | 694.3189 | 2 | 347.7 | 4.153 |
| | ND | 111:120 | 1 | 1107.604 | 2 | 554.3 | 46.78 |
| | ND | 17:27 | 1 | 1164.572 | 2 | 582.8 | 44.71 |

To aid the selection of the most appropriate transitions for each peptide, previous empirical observations of the peptides in a public repository of tandem mass spectra (the Peptide Atlas [http://www.peptideatlas.org/]) and/or during the preceding discovery exercise (described above) were reviewed.

As a preferred but not limiting method an intelligent SRM (iSRM) method was set up consisting of a combination of so-called primary and secondary transitions. In such an approach, when all primary transitions relative to a given peptide are detected above a defined threshold, secondary transitions are then triggered to help confirming the identity of the targeted molecule. Here, the approach aimed to reduce the number of transitions to be continuously monitored in the assay and evaluate the peptide detection level in a particular matrix. Two primary and 6 secondary transitions were selected as reported in Table 15.

TABLE 15
List of transitions for use in SRM methods for diagnosis and/or prognostic monitoring of a subject with acute brain damage

| | | Collision | | | | | Precursor | Product | |
|---|---|---|---|---|---|---|---|---|---|
| | | Energy | | Primary (0)/ | Peptide | | charge | charge | Ion |
| Q1 (m/z) | Q3 (m/z) | (V) | S-lens (V) | Secondary (1) | sequence | Protein description | state | state | type |
| 337.677 | 272.124 | 15 | 68 | 1 | AAEEVR | sp\|P07108\|ACBP_HUMAN Acyl-CoA-binding protein | 2 | 1 | b3 |
| 337.677 | 274.187 | 15 | 68 | 1 | | OS = Homo sapiens GN = DBI PE = 1 SV = 2 | 2 | 1 | y2 |
| 337.677 | 302.158 | 15 | 68 | 1 | | | 2 | 2 | y5 |
| 337.677 | 401.166 | 15 | 68 | 0 | | | 2 | 1 | b4 |
| 337.677 | 403.229 | 15 | 68 | 0 | | | 2 | 1 | y3 |
| 337.677 | 500.235 | 15 | 68 | 1 | | | 2 | 1 | b5 |
| 337.677 | 532.272 | 15 | 68 | 0 | | | 2 | 1 | y4 |
| 337.677 | 603.309 | 15 | 68 | 0 | | | 2 | 1 | y5 |
| 437.717 | 345.224 | 18 | 98 | 1 | GEEVDVAR | sp\|O94760\|DDAH1_HUMAN N(G); N(G)-dimethylarginine | 2 | 1 | y3 |
| 437.717 | 415.182 | 18 | 98 | 1 | | dimethylaminohydrolase 1 OS = Homo sapiens | 2 | 1 | b4 |
| 437.717 | 460.251 | 18 | 98 | 0 | | GN = DDAH1 PE = 1 SV = 3 | 2 | 1 | y4 |
| 437.717 | 530.209 | 18 | 98 | 0 | | | 2 | 1 | b5 |
| 437.717 | 559.319 | 18 | 98 | 0 | | | 2 | 1 | y5 |
| 437.717 | 629.277 | 18 | 98 | 1 | | | 2 | 1 | b6 |
| 437.717 | 688.362 | 18 | 98 | 0 | | | 2 | 1 | y6 |
| 437.717 | 700.314 | 18 | 98 | 1 | | | 2 | 1 | b7 |
| 443.274 | 411.26 | 18 | 100 | 0 | VLTPTQVK | sp\|P30086\|PEBP1_HUMAN Phosphatidylethanolamine-binding | 2 | 1 | b4 |
| 443.274 | 475.287 | 18 | 100 | 1 | | protein 1 OS = Homo sapiens | 2 | 1 | y4 |
| 443.274 | 512.307 | 18 | 100 | 1 | | GN = PEBP1 PE = 1 SV = 3 | 2 | 1 | b5 |
| 443.274 | 572.34 | 18 | 100 | 0 | | | 2 | 1 | y5 |
| 443.274 | 640.366 | 18 | 100 | 1 | | | 2 | 1 | b6 |
| 443.274 | 673.387 | 18 | 100 | 0 | | | 2 | 1 | y6 |
| 443.274 | 739.434 | 18 | 100 | 0 | | | 2 | 1 | b7 |
| 443.274 | 786.471 | 18 | 100 | 1 | | | 2 | 1 | y7 |
| 462.708 | 382.135 | 19 | 105 | 1 | HDELTYF | sp\|P04080\|CYTB_HUMAN Cystatin-B | 2 | 1 | b3 |
| 462.708 | 430.197 | 19 | 105 | 1 | | OS = Homo sapiens GN = CSTB PE = 1 SV = 2 | 2 | 1 | y3 |
| 462.708 | 495.219 | 19 | 105 | 0 | | | 2 | 1 | b4 |
| 462.708 | 543.281 | 19 | 105 | 1 | | | 2 | 1 | y4 |
| 462.708 | 596.267 | 19 | 105 | 0 | | | 2 | 1 | b5 |
| 462.708 | 672.323 | 19 | 105 | 0 | | | 2 | 1 | y5 |
| 462.708 | 759.33 | 19 | 105 | 0 | | | 2 | 1 | b6 |
| 462.708 | 787.35 | 19 | 105 | 1 | | | 2 | 1 | y6 |
| 469.253 | 277.154 | 19 | 107 | 1 | LYEQLSGK | sp\|P30086\|PEBP1_HUMAN Phosphatidylethanolamine-binding | 2 | 1 | b2 |
| 469.253 | 291.166 | 19 | 107 | 1 | | protein 1 OS = Homo sapiens | 2 | 1 | y3 |
| 469.253 | 534.255 | 19 | 107 | 1 | | GN = PEBP1 PE = 1 SV = 3 | 2 | 1 | b4 |
| 469.253 | 647.339 | 19 | 107 | 1 | | | 2 | 1 | b5 |
| 469.253 | 661.351 | 19 | 107 | 0 | | | 2 | 1 | y6 |
| 469.253 | 734.371 | 19 | 107 | 1 | | | 2 | 1 | b6 |
| 469.253 | 791.393 | 19 | 107 | 1 | | | 2 | 1 | b7 |
| 469.253 | 824.414 | 19 | 107 | 0 | | | 2 | 1 | y7 |
| 485.221 | 347.138 | 20 | 112 | 1 | MSQAEFEK | sp\|P07108\|ACBP_HUMAN Acyl-CoA-binding | 2 | 1 | b3 |
| 485.221 | 419.7 | 20 | 112 | 1 | | protein OS = Homo sapiens GN = DBI PE = 1 SV = 2 | 2 | 2 | y7 |
| 485.221 | 423.223 | 20 | 112 | 0 | | | 2 | 1 | y3 |
| 485.221 | 547.218 | 20 | 112 | 1 | | | 2 | 1 | b5 |
| 485.221 | 552.266 | 20 | 112 | 1 | | | 2 | 1 | y4 |
| 485.221 | 623.303 | 20 | 112 | 0 | | | 2 | 1 | y5 |
| 485.221 | 751.362 | 20 | 112 | 0 | | | 2 | 1 | y6 |
| 485.221 | 838.394 | 20 | 112 | 0 | | | 2 | 1 | y7 |
| 517.772 | 446.26 | 21 | 122 | 1 | GLESTTLADK | sp\|P21291\|CSRP1_HUMAN Cysteine and | 2 | 1 | y4 |
| 517.772 | 488.235 | 21 | 122 | 1 | | glycine-rich protein 1 OS = Homo sapiens | 2 | 1 | b5 |
| 517.772 | 547.308 | 21 | 122 | 1 | | GN = CSRP1 PE = 1 SV = 3 | 2 | 1 | y5 |
| 517.772 | 648.356 | 21 | 122 | 1 | | | 2 | 1 | y6 |
| 517.772 | 702.366 | 21 | 122 | 1 | | | 2 | 1 | b7 |
| 517.772 | 735.388 | 21 | 122 | 0 | | | 2 | 1 | y7 |
| 517.772 | 773.403 | 21 | 122 | 1 | | | 2 | 1 | b8 |
| 517.772 | 864.43 | 21 | 122 | 0 | | | 2 | 1 | y8 |
| 531.241 | 476.235 | 21 | 126 | 1 | GGEAAEAEAEK | sp\|P25713\|MT3_HUMAN Metallothionein-3 | 2 | 1 | y4 |
| 531.241 | 515.209 | 21 | 126 | 1 | | OS = Homo sapiens GN = MT3 PE = 1 SV = 1 | 2 | 1 | b6 |
| 531.241 | 547.272 | 21 | 126 | 0 | | | 2 | 1 | y5 |
| 531.241 | 586.246 | 21 | 126 | 1 | | | 2 | 1 | b7 |
| 531.241 | 676.314 | 21 | 126 | 0 | | | 2 | 1 | y6 |
| 531.241 | 747.351 | 21 | 126 | 0 | | | 2 | 1 | y7 |
| 531.241 | 818.389 | 21 | 126 | 0 | | | 2 | 1 | y8 |
| 531.241 | 916.369 | 21 | 126 | 1 | | | 2 | 1 | b10 |
| 531.256 | 503.282 | 21 | 126 | 1 | WDAWNELK | sp\|P07108\|ACBP_HUMAN Acyl-CoA-binding protein | 2 | 1 | y4 |
| 531.256 | 559.229 | 21 | 126 | 1 | | OS = Homo sapiens GN = DBI PE = 1 SV = 2 | 2 | 1 | b4 |
| 531.256 | 673.272 | 21 | 126 | 1 | | | 2 | 1 | b5 |
| 531.256 | 689.361 | 21 | 126 | 0 | | | 2 | 1 | y5 |
| 531.256 | 760.398 | 21 | 126 | 0 | | | 2 | 1 | y6 |
| 531.256 | 802.315 | 21 | 126 | 1 | | | 2 | 1 | b6 |
| 531.256 | 875.425 | 21 | 126 | 1 | | | 2 | 1 | y7 |
| 531.256 | 915.399 | 21 | 126 | 1 | | | 2 | 1 | b7 |
| 532.785 | 555.313 | 21 | 126 | 1 | GAEILADTFK | sp\|O94760\|DDAH1_HUMAN N(G); N(G)-dimethylarginine | 2 | 1 | b6 |
| 532.785 | 581.292 | 21 | 126 | 1 | | dimethylaminohydrolase 1 OS = Homo sapiens | 2 | 1 | y5 |
| 532.785 | 670.34 | 21 | 126 | 1 | | GN = DDAH1 PE = 1 SV = 3 | 2 | 1 | b7 |
| 532.785 | 694.377 | 21 | 126 | 1 | | | 2 | 1 | y6 |
| 532.785 | 771.388 | 21 | 126 | 0 | | | 2 | 1 | b8 |
| 532.785 | 807.461 | 21 | 126 | 0 | | | 2 | 1 | y7 |
| 532.785 | 918.456 | 21 | 126 | 1 | | | 2 | 1 | b9 |
| 532.785 | 936.503 | 21 | 126 | 1 | | | 2 | 1 | y8 |
| 535.255 | 423.223 | 22 | 127 | 1 | MPAFAEFEK | sp\|P07108-3\|ACBP_HUMAN Isoform 3 of Acyl-CoA-binding | 2 | 1 | y3 |
| 535.255 | 447.206 | 22 | 127 | 1 | | protein OS = Homo sapiens GN = DBI | 2 | 1 | b4 |
| 535.255 | 469.734 | 22 | 127 | 1 | | | 2 | 2 | y8 |
| 535.255 | 552.266 | 22 | 127 | 1 | | | 2 | 1 | y4 |
| 535.255 | 623.303 | 22 | 127 | 0 | | | 2 | 1 | y5 |
| 535.255 | 770.371 | 22 | 127 | 0 | | | 2 | 1 | y6 |
| 535.255 | 841.409 | 22 | 127 | 0 | | | 2 | 1 | y7 |
| 535.255 | 938.461 | 22 | 127 | 0 | | | 2 | 1 | y8 |
| 575.767 | 457.192 | 23 | 139 | 1 | TPEEYPESAK | sp\|O94760\|DDAH1_HUMAN N(G); N(G)-dimethylarginine | 2 | 1 | b4 |
| 575.767 | 531.277 | 23 | 139 | 1 | | dimethylaminohydrolase 1 OS = Homo sapiens | 2 | 1 | y5 |
| 575.767 | 620.256 | 23 | 139 | 1 | | GN = DDAH1 PE = 1 SV = 3 | 2 | 1 | b5 |
| 575.767 | 694.34 | 23 | 139 | 0 | | | 2 | 1 | y6 |
| 575.767 | 823.383 | 23 | 139 | 0 | | | 2 | 1 | y7 |
| 575.767 | 846.351 | 23 | 139 | 1 | | | 2 | 1 | b7 |
| 575.767 | 933.383 | 23 | 139 | 1 | | | 2 | 1 | b8 |
| 575.767 | 952.425 | 23 | 139 | 1 | | | 2 | 1 | y8 |
| 646.359 | 359.24 | 25 | 160 | 1 | ALPESLGQHALR | sp\|O94760\|DDAH1_HUMAN N(G); N(G)-dimethylarginine | 2 | 1 | y3 |
| 646.359 | 441.251 | 25 | 160 | 1 | | dimethylaminohydrolase 1 OS = Homo sapiens | 2 | 2 | y8 |
| 646.359 | 554.299 | 25 | 160 | 0 | | GN = DDAH1 PE = 1 SV = 3 | 2 | 2 | y10 |
| 646.359 | 624.357 | 25 | 160 | 1 | | | 2 | 1 | y5 |
| 646.359 | 681.379 | 25 | 160 | 0 | | | 2 | 1 | y6 |
| 646.359 | 881.495 | 25 | 160 | 1 | | | 2 | 1 | y8 |
| 646.359 | 933.478 | 25 | 160 | 1 | | | 2 | 1 | b9 |
| 646.359 | 1107.59 | 25 | 160 | 1 | | | 2 | 1 | y10 |
| 663.856 | 485.271 | 26 | 165 | 1 | SQVVAGTNYFIK | sp\|P04080\|CYTB_HUMAN Cystatin-B | 2 | 1 | b5 |
| 663.856 | 785.419 | 26 | 165 | 1 | | OS = Homo sapiens GN = CSTB PE = 1 SV = 2 | 2 | 1 | y6 |
| 663.856 | 842.44 | 26 | 165 | 0 | | | 2 | 1 | y7 |
| 663.856 | 913.477 | 26 | 165 | 0 | | | 2 | 1 | y8 |
| 663.856 | 1012.546 | 26 | 165 | 1 | | | 2 | 1 | y9 |
| 663.856 | 1067.515 | 26 | 165 | 1 | | | 2 | 1 | b10 |
| 663.856 | 1111.614 | 26 | 165 | 1 | | | 2 | 1 | y10 |
| 663.856 | 1180.599 | 26 | 165 | 1 | | | 2 | 1 | b11 |
| 664.87 | 507.267 | 26 | 165 | 1 | QHQLYVGVLGSK | sp\|O94760\|DDAH1_HUMAN N(G); N(G)-dimethylarginine | 2 | 1 | b4 |
| 664.87 | 560.34 | 26 | 165 | 1 | | dimethylaminohydrolase 1 OS = Homo sapiens | 2 | 1 | y6 |
| 664.87 | 659.408 | 26 | 165 | 1 | | GN = DDAH1 PE = 1 SV = 3 | 2 | 1 | y7 |
| 664.87 | 670.33 | 26 | 165 | 1 | | | 2 | 1 | b5 |
| 664.87 | 769.399 | 26 | 165 | 1 | | | 2 | 1 | b6 |
| 664.87 | 822.471 | 26 | 165 | 0 | | | 2 | 1 | y8 |
| 664.87 | 935.556 | 26 | 165 | 1 | | | 2 | 1 | y9 |
| 664.87 | 1063.614 | 26 | 165 | 0 | | | 2 | 1 | y10 |
| 711.86 | 752.32 | 28 | 179 | 1 | VHVGDEDFVHLR | sp\|P04080\|CYTB_HUMAN Cystatin-B | 2 | 1 | b7 |
| 711.86 | 786.425 | 28 | 179 | 1 | | OS = Homo sapiens GN = CSTB PE = 1 SV = 2 | 2 | 1 | y6 |
| 711.86 | 915.468 | 28 | 179 | 1 | | | 2 | 1 | y7 |
| 711.86 | 998.457 | 28 | 179 | 1 | | | 2 | 1 | b9 |
| 711.86 | 1030.495 | 28 | 179 | 1 | | | 2 | 1 | y8 |
| 711.86 | 1087.516 | 28 | 179 | 0 | | | 2 | 1 | y9 |
| 711.86 | 1135.516 | 28 | 179 | 1 | | | 2 | 1 | b10 |
| 711.86 | 1186.585 | 28 | 179 | 0 | | | 2 | 1 | y10 |
| 717.344 | 783.399 | 28 | 181 | 1 | GFGFGQGAGALVHSE | sp\|P21291\|CSRP1_HUMAN Cysteine | 2 | 1 | y8 |
| 717.344 | 840.421 | 28 | 181 | 0 | | and glycine-rich protein 1 OS = Homo sapiens | 2 | 1 | y9 |
| 717.344 | 850.384 | 28 | 181 | 1 | | GN = CSRP1 PE = 1 SV = 3 | 2 | 1 | b10 |
| 717.344 | 963.468 | 28 | 181 | 1 | | | 2 | 1 | b11 |
| 717.344 | 968.479 | 28 | 181 | 1 | | | 2 | 1 | y10 |
| 717.344 | 1025.5 | 28 | 181 | 0 | | | 2 | 1 | y11 |
| 717.344 | 1062.536 | 28 | 181 | 1 | | | 2 | 1 | b12 |
| 717.344 | 1199.595 | 28 | 181 | 1 | | | 2 | 1 | b13 |
| 717.356 | 561.292 | 35 | 181 | 1 | QATVGDINTERPGMLDFTGK | sp\|P07108\|ACBP_HUMAN Acyl-CoA-binding | 3 | 2 | y10 |
| 717.356 | 676.337 | 35 | 181 | 1 | | protein OS = Homo sapiens GN = DBI PE = 1 SV = 2 | 3 | 2 | y12 |
| 717.356 | 789.901 | 35 | 181 | 1 | | | 3 | 2 | y14 |
| 717.356 | 847.414 | 35 | 181 | 1 | | | 3 | 2 | y15 |
| 717.356 | 875.925 | 35 | 181 | 0 | | | 3 | 2 | y16 |
| 717.356 | 925.459 | 35 | 181 | 1 | | | 3 | 2 | y17 |
| 717.356 | 965.476 | 35 | 181 | 1 | | | 3 | 1 | y9 |
| 717.356 | 975.983 | 35 | 181 | 0 | | | 3 | 2 | y18 |
| 737.844 | 721.372 | 28 | 187 | 1 | GYGYGQGAGTLSTDK | sp\|P21291\|CSRP1_HUMAN Cysteine | 2 | 1 | y7 |
| 737.844 | 754.315 | 28 | 187 | 1 | | and glycine-rich protein 1 OS = Homo sapiens | 2 | 1 | b8 |
| 737.844 | 792.409 | 28 | 187 | 0 | | GN = CSRP1 PE = 1 SV = 3 | 2 | 1 | y8 |
| 737.844 | 849.431 | 28 | 187 | 1 | | | 2 | 1 | y9 |
| 737.844 | 977.489 | 28 | 187 | 1 | | | 2 | 1 | y10 |
| 737.844 | 1034.511 | 28 | 187 | 0 | | | 2 | 1 | y11 |
| 737.844 | 1254.596 | 28 | 187 | 1 | | | 2 | 1 | y13 |
| 737.844 | 1328.575 | 28 | 187 | 1 | | | 2 | 1 | b14 |
| 774.256 | 436.186 | 30 | 198 | 1 | SC[Carboxyamidomethyl]C[Carboxyamidomethyl] | sp\|P25713\|MT3_HUMAN | 2 | 1 | y3 |
| 774.256 | 650.725 | 30 | 198 | 1 | SC[Carboxyamidomethyl]C[Carboxyamidomethyl] | Metallothionein-3 OS = Homo sapiens | 2 | 2 | y10 |
| 774.256 | 733.318 | 30 | 198 | 0 | PAEC[Carboxyamidomethyl] | GN = MT3 PE = 1 SV = 1 | 2 | 1 | y6 |
| 774.256 | 893.349 | 30 | 198 | 0 | EK | | 2 | 1 | y7 |
| 774.256 | 1053.379 | 30 | 198 | 1 | | | 2 | 1 | y8 |
| 774.256 | 1112.326 | 30 | 198 | 1 | | | 2 | 1 | b9 |
| 774.256 | 1140.411 | 30 | 198 | 1 | | | 2 | 1 | y9 |
| 774.256 | 1300.442 | 30 | 198 | 1 | | | 2 | 1 | y10 |
| 780.917 | 757.347 | 30 | 200 | 1 | LYTLVLTDPDAPSR | sp\|P30086\|PEBP1_HUMAN Phosphatidylethanolamine-binding | 2 | 1 | y7 |
| 780.917 | 804.486 | 30 | 200 | 1 | | protein 1 OS = Homo sapiens GN = PEBP1 PE = 1 SV = 3 | 2 | 1 | b7 |
| 780.917 | 858.395 | 30 | 200 | 0 | | | 2 | 1 | y8 |
| 780.917 | 919.513 | 30 | 200 | 1 | | | 2 | 1 | b8 |
| 780.917 | 971.479 | 30 | 200 | 0 | | | 2 | 1 | y9 |
| 780.917 | 1070.547 | 30 | 200 | 1 | | | 2 | 1 | y10 |
| 780.917 | 1131.593 | 30 | 200 | 1 | | | 2 | 1 | b10 |
| 780.917 | 1202.63 | 30 | 200 | 1 | | | 2 | 1 | b11 |
| 816.902 | 942.479 | 31 | 211 | 1 | NRPTSISWDGLDSGK | sp\|P30086\|PEBP1_HUMAN Phosphatidylethanolamine-binding | 2 | 1 | b8 |
| 816.902 | 964.437 | 31 | 211 | 1 | | protein 1 OS = Homo sapiens GN = PEBP1 PE = 1 SV = 3 | 2 | 1 | y9 |
| 816.902 | 1057.506 | 31 | 211 | 0 | | | 2 | 1 | b9 |
| 816.902 | 1227.611 | 31 | 211 | 1 | | | 2 | 1 | b11 |
| 816.902 | 1342.638 | 31 | 211 | 0 | | | 2 | 1 | b12 |
| 816.902 | 1362.653 | 31 | 211 | 1 | | | 2 | 1 | y13 |
| 816.902 | 1429.67 | 31 | 211 | 1 | | | 2 | 1 | b13 |
| 816.902 | 1486.692 | 31 | 211 | 1 | | | 2 | 1 | b14 |
| 840.397 | 921.478 | 32 | 217 | 1 | DENATLDGGDVLFTGR | sp\|O94760\|DDAH1_HUMAN N(G); N(G)-dimethylarginine | 2 | 1 | y9 |
| 840.397 | 1036.505 | 32 | 217 | 0 | | dimethylaminohydrolase 1 | 2 | 1 | y10 |
| 840.397 | 1087.453 | 32 | 217 | 1 | | OS = Homo sapiens GN = DDAH1 PE = 1 SV = 3 | 2 | 1 | b11 |
| 840.397 | 1149.589 | 32 | 217 | 0 | | | 2 | 1 | y11 |
| 840.397 | 1200.537 | 32 | 217 | 1 | | | 2 | 1 | b12 |
| 840.397 | 1250.637 | 32 | 217 | 1 | | | 2 | 1 | y12 |
| 840.397 | 1347.606 | 32 | 217 | 1 | | | 2 | 1 | b13 |
| 840.397 | 1448.653 | 32 | 217 | 1 | | | 2 | 1 | b14 |
| 842.949 | 932.472 | 32 | 218 | 1 | DYAVSTVPVADGLHLK | sp\|O94760\|DDAH1_HUMAN N(G); N(G)-dimethylarginine | 2 | 1 | b9 |
| 842.949 | 949.546 | 32 | 218 | 0 | | dimethylaminohydrolase 1 OS = Homo sapiens | 2 | 1 | y9 |
| 842.949 | 1003.509 | 32 | 218 | 1 | | GN = DDAH1 PE = 1 SV = 3 | 2 | 1 | b10 |
| 842.949 | 1048.614 | 32 | 218 | 1 | | | 2 | 1 | y10 |
| 842.949 | 1118.536 | 32 | 218 | 1 | | | 2 | 1 | b11 |
| 842.949 | 1149.662 | 32 | 218 | 1 | | | 2 | 1 | y11 |
| 842.949 | 1236.694 | 32 | 218 | 0 | | | 2 | 1 | y12 |
| 842.949 | 1335.763 | 32 | 218 | 1 | | | 2 | 1 | y13 |
| 854.956 | 513.35 | 32 | 222 | 1 | YVWLVYEQDRPLK | sp\|P30086\|PEBP1_HUMAN Phosphatidylethanolamine-binding | 2 | 1 | y4 |
| 854.956 | 756.436 | 32 | 222 | 1 | | protein 1 OS = Homo sapiens GN = PEBP1 PE = 1 SV = 3 | 2 | 1 | y6 |
| 854.956 | 885.478 | 32 | 222 | 1 | | | 2 | 1 | y7 |
| 854.956 | 1048.542 | 32 | 222 | 0 | | | 2 | 1 | y8 |
| 854.956 | 1147.61 | 32 | 222 | 0 | | | 2 | 1 | y9 |
| 854.956 | 1196.562 | 32 | 222 | 1 | | | 2 | 1 | b9 |
| 854.956 | 1260.694 | 32 | 222 | 1 | | | 2 | 1 | y10 |
| 854.956 | 1352.663 | 32 | 222 | 1 | | | 2 | 1 | b10 |
| 906.435 | 423.223 | 43 | 237 | 1 | MWGDLWLLPPASANPGTGTEAEFEK | sp\|P07108-2\|ACBP_HUMAN Isoform 2 of Acyl-CoA-binding | 3 | 1 | y3 |
| 906.435 | 490.175 | 43 | 237 | 1 | | protein OS = Homo sapiens GN = DBI | 3 | 1 | b4 |
| 906.435 | 623.303 | 43 | 237 | 0 | | | 3 | 1 | y5 |
| 906.435 | 789.338 | 43 | 237 | 0 | | | 3 | 1 | b6 |
| 906.435 | 910.415 | 43 | 237 | 0 | | | 3 | 1 | y8 |
| 906.435 | 1058.023 | 43 | 237 | 1 | | | 3 | 2 | y20 |
| 906.435 | 1165.537 | 43 | 237 | 1 | | | 3 | 1 | y11 |
| 906.435 | 1200.589 | 43 | 237 | 0 | | | 3 | 2 | y23 |
| 975.476 | 542.293 | 36 | 258 | 0 | GNDISSGTVLSDYVGSGPPK | sp\|P30086\|PEBP1_HUMAN Phosphatidylethanolamine-binding | 2 | 1 | y6 |
| 975.476 | 919.451 | 36 | 258 | 1 | | protein 1 OS = Homo sapiens GN = PEBP1 PE = 1 SV = 3 | 2 | 1 | y9 |
| 975.476 | 1006.483 | 36 | 258 | 1 | | | 2 | 1 | y10 |
| 975.476 | 1119.568 | 36 | 258 | 0 | | | 2 | 1 | y11 |
| 975.476 | 1218.636 | 36 | 258 | 1 | | | 2 | 1 | y12 |
| 975.476 | 1376.705 | 36 | 258 | 1 | | | 2 | 1 | y14 |
| 975.476 | 1408.659 | 36 | 258 | 1 | | | 2 | 1 | b14 |
| 975.476 | 1463.737 | 36 | 258 | 1 | | | 2 | 1 | y15 |
| 979.472 | 864.9 | 37 | 259 | 0 | TKPSDEEMLFIYGHYK | sp\|P07108\|ACBP_HUMAN Acyl-CoA-binding | 2 | 2 | y14 |
| 979.472 | 927.472 | 37 | 259 | 0 | | protein OS = Homo sapiens GN = DBI PE = 1 SV = 2 | 2 | 1 | y7 |
| 979.472 | 1031.471 | 37 | 259 | 1 | | | 2 | 1 | b9 |
| 979.472 | 1171.596 | 37 | 259 | 1 | | | 2 | 1 | y9 |
| 979.472 | 1178.539 | 37 | 259 | 1 | | | 2 | 1 | b10 |
| 979.472 | 1291.623 | 37 | 259 | 1 | | | 2 | 1 | b11 |
| 979.472 | 1429.682 | 37 | 259 | 1 | | | 2 | 1 | y11 |
| 979.472 | 1454.687 | 37 | 259 | 1 | | | 2 | 1 | b12 |
| 1188.92 | 509.202 | 44 | 316 | 1 | MDPETC[Carboxyamidomethyl]PC[Carboxyamidomethyl] | sp\|P25713\|MT3_HUMAN Metallothionein-3 | 2 | 1 | y4 |
| 1188.92 | 740.27 | 44 | 316 | 1 | PSGGSC[Carboxyamidomethyl] | OS = Homo sapiens GN = MT3 PE = 1 SV = 1 | 2 | 1 | y6 |
| 1188.92 | 831.301 | 44 | 316 | 0 | TC[Carboxyamidomethyl]ADSC | | 2 | 1 | b7 |
| 1188.92 | 841.317 | 44 | 316 | 1 | [Carboxyamidomethyl]K | | 2 | 1 | y7 |
| 1188.92 | 1001.348 | 44 | 316 | 0 | | | 2 | 1 | y8 |
| 1188.92 | 1202.423 | 44 | 316 | 1 | | | 2 | 1 | y11 |
| 1188.92 | 1289.455 | 44 | 316 | 0 | | | 2 | 1 | y12 |
| 1188.92 | 1386.508 | 44 | 316 | 0 | | | 2 | 1 | y13 |
| 1229.64 | 854.4 | 45 | 316 | 1 | VFQSLPHENKPLTLSNYQTNK | sp\|P04080\|CYTB_HUMAN Cystatin-B | 2 | 1 | y7 |
| 1229.64 | 938.473 | 45 | 316 | 1 | | OS = Homo sapiens GN = CSTB PE = 1 SV = 2 | 2 | 1 | b8 |
| 1229.64 | 942.484 | 45 | 316 | 0 | | | 2 | 2 | y16 |
| 1229.64 | 1068.531 | 45 | 316 | 1 | | | 2 | 1 | y9 |
| 1229.64 | 1099.565 | 45 | 316 | 1 | | | 2 | 2 | b19 |
| 1229.64 | 1156.587 | 45 | 316 | 1 | | | 2 | 2 | b20 |
| 1229.64 | 1180.61 | 45 | 316 | 1 | | | 2 | 1 | b10 |
| 1229.64 | 1278.668 | 45 | 316 | 0 | | | 2 | 1 | y11 |

When no data was available, prediction from SRM Atlas or Pinpoint software (Thermo Scientific) was used to choose the transitions. In that case, 4 primary and 4 secondary transitions were selected as reported in Table 15. The S-lens parameters for each precursor-ion were set-up according to m/z values and previous experimental data. Collision energies were determined by Pinpoint using a pre-defined calculation. The chosen cycle time was 1.6 s to monitor 80 primary transitions. A total of 240 transitions were used to monitor the 30 signature-peptides. The scan time of the triggered transitions was 0.2 s. A TSQ Vantage mass spectrometer (Thermo Scientific) was used using Q1 peak width (FWHM) of 0.7 and argon pressure in the collision cell of 1.2 mTorr. Positive ionisation was used. Capillary temperature, vaporizer, sheath gas and auxiliary gas were optimized for maximal ion sensitivities.

A reversed-phase liquid chromatography (RP-LC) separation was implemented before MS. Peptide separation occurred on a 50×1 mm column at 100 μL/min with a 13.25 min gradient of 30% CH₃CN. A Finnigan Surveyor MS Pump Plus LC system (Thermo Scientific) was used.

### Testing of the Method

To demonstrate the presence of the target proteins in a more readily accessible sample, the developed MRM method was evaluated on human plasma sample digested with trypsin. Briefly, a volume of 30 μL plasma (Dade Behring) was added to 1680 μL triethylammonium hydrogen carbonate buffer (TEAB) 100 mM and 90 μL sodium dodecyl sulfate 1%. Reduction was performed at 55° C. for 1 h with tris(2-carboxyethyl) phosphine hydrochloride 20 mM (95.4 μL). A volume of 90 μL iodoacetamide 150 mM was then added for 1 h reaction in the dark at room temperature. A volume of 180 μL trypsin (Promega) 0.4 μg/μL in TEAB was added. Digestion was performed overnight at 37° C. Sample purification was first performed with Hypersep C18 500 mg (Thermo Scientific). Strong cation-exchange cartridges were used for further purification. The sample was divided into three aliquots. Aliquots were re-suspended in 500 μL 3% CH₃CN, 0.2% formic acid, 0.2 mg/mL glucagon before RP-LC SRM analysis. Twenty μL were used per RP-LC iSRM analysis. Data analysis was carried out using Pinpoint.

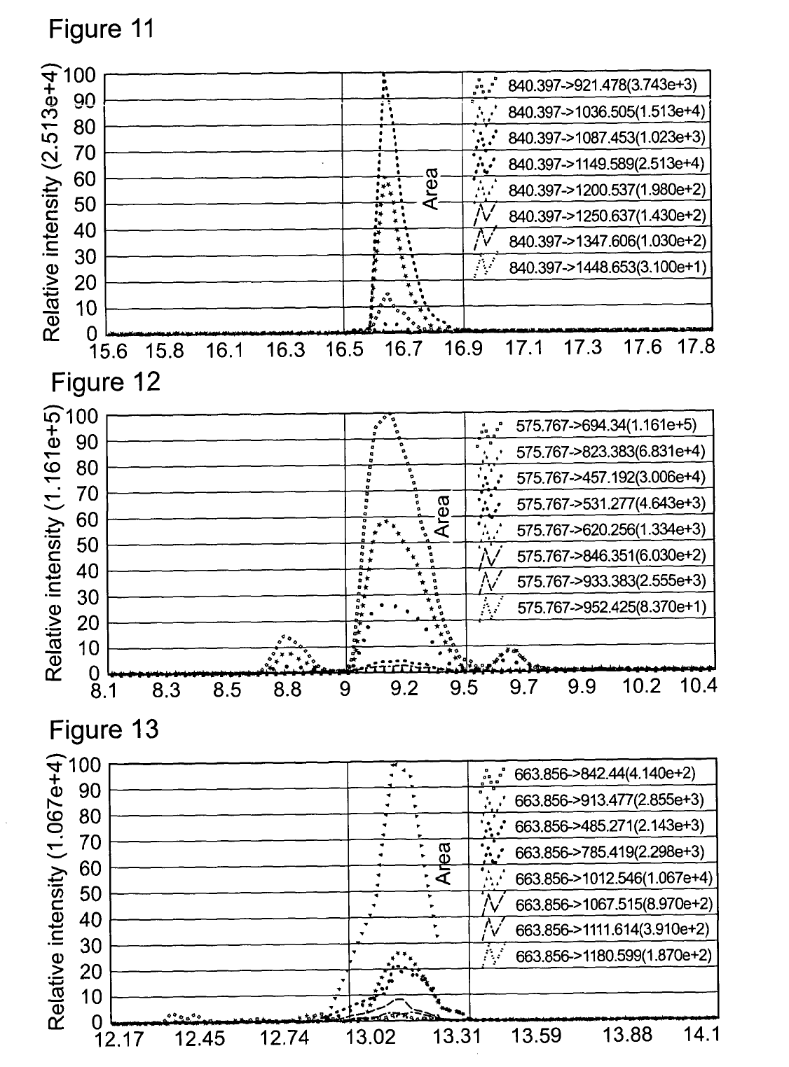

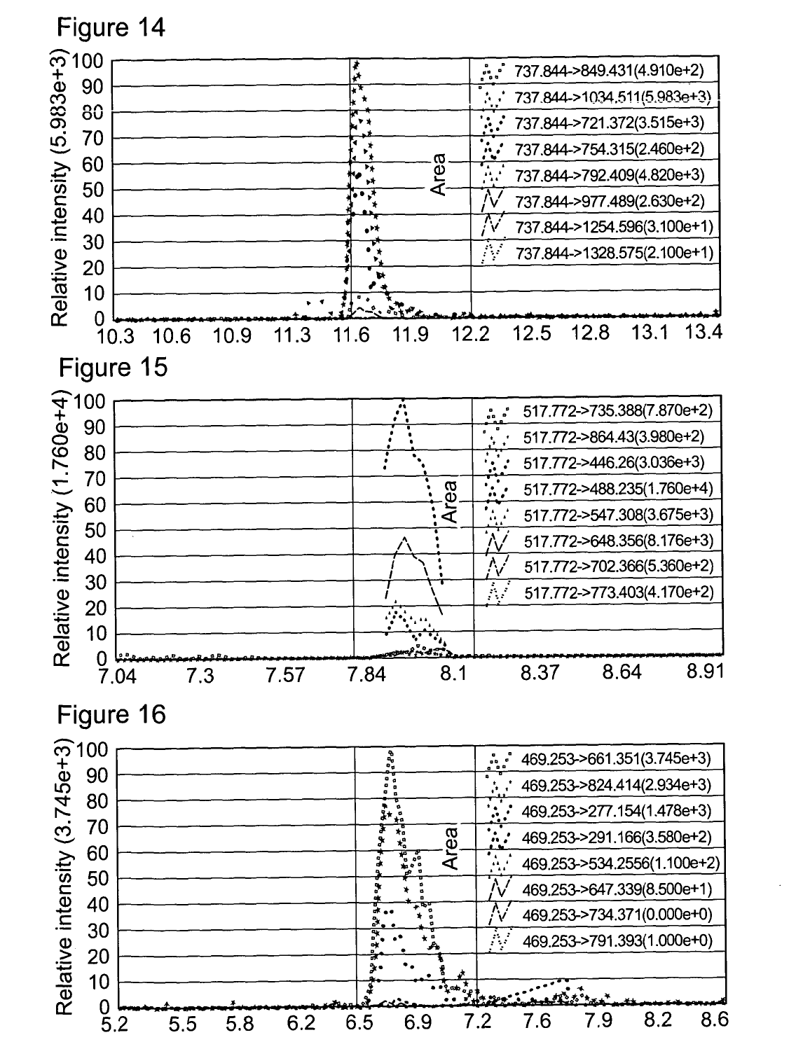

FIGS. 11 to 16 show chromatograms of the iSRM signals of transitions for signature-peptides DENATLDGGDVLFTGR, TPEEYPESAK, SQVVAGTNYFIK, GYGYGQGAGTLSTDK, GLESTTLADK and LYEQLSGK representative of proteins DDAH1, CYTB, CSRP1 and PEBP1.

Thus it is demonstrated that the SRM method developed herein allows for the monitoring of 30 signature-peptides representative of 6 stroke biomarker candidates. Proof-of-principle of the method applicability was demonstrated in a plasma sample digested with trypsin. The method could be applied to several sample matrixes.

The demonstrations in this example were carried out using the markers of Panel A. As noted above, this is illustrative of this mode of detection. This mode of detection may be applied equally to any other of the markers or groups of markers disclosed in this document. To work the invention using those other marker(s) according to this mode of detection, the skilled worker simply follows the guidance given above but substitutes their selected other marker(s) for those of Panel A.

### REFERENCES

1. Foerch, C., Montaner, J., Furie, K. L., Ning, M. M., and Lo, E. H. (2009) Invited Article: Searching for oracles? Blood biomarkers in acute stroke. Neurology 73, 393-399.
2. Poca, M. A., Sahuquillo, J., Vilalta, A., De los Rios, J., Robles, A., and Exposito, L. (2006) Percutaneous implantation of cerebral microdialysis catheters by twist-drill craniostomy in neurocritical patients: Description of the technique and results of a feasibility study in 97 patients. J. Neurotrauma 23, 1510-1517.
3. Hutchinson, P. J., O'Connell, M. T., Kirkpatrick, P. J., and Pickard, J. D. (2002) How can we measure substrate, metabolite and neurotransmitter concentrations in the human brain? Physiol. Meas. 23, R75-R109.
4. Reinstrup, P., Stahl, N., Mellergard, P., Uski, T., Ungerstedt, U., and Nordstrom, C. H. (2000) Intracerebral microdialysis in clinical practice: Baseline values for chemical markers during wakefulness, anesthesia, and neurosurgery. Neurosurgery 47, 701-709.
5. Tisdall, M. M., and Smith, M. (2006) Cerebral microdialysis: research technique or clinical tool. Br. J. Anaesth. 97, 18-25.
6. Maurer, M. H. (2008) Proteomics of brain extracellular fluid (ECF) and cerebrospinal fluid (CSF). Mass Spectrom. Rev., DOI: 10.1002/mas.20213.
7. Maurer, M. H., Haux, D., Unterberg, A. W., and Sakowitz, O. W. (2008) Proteomics of human cerebral microdialysate: From detection of biomarkers to clinical application. Proteomics Clin. Appl. 2, 437-443.
8. Helmy, A., Carpenter, K. L. H., Skepper, J. N., Kirkpatrick, P. J., Pickard, J. D., and Hutchinson, P. J. (2009) Microdialysis of Cytokines: Methodological Considerations, Scanning Electron Microscopy, and Determination of Relative Recovery. J. Neurotrauma 26, 549-561.
9. Donato, R. (2001) S100: a multigenic family of calcium-modulated proteins of the EF-hand type with intracellular and extracellular functional roles. Int. J. Biochem. Cell Biol. 33, 637-668.
10. Afinowi, R., Tisdall, M., Keir, G., Smith, M., Kitchen, N., and Petzold, A. (2009) Improving the recovery of S100B protein in cerebral microdialysis: Implications for multimodal monitoring in neurocritical care. J. Neurosci. Methods 181, 95-99.
11. Maurer, M. H., Berger, C., Wolf, M., Futterer, C. D., Feldmann, R. E., Jr., Schwab, S., and Kuschinsky, W. (2003) The proteome of human brain microdialysate, Proteome Science 1, 7.
12. Maurer, M. H., Haux, D., Sakowitz, O. W., Unterberg, A. W., and Kuschinsky, W. (2007) Identification of early markers for symptomatic vasospasm in human cerebral microdialysate after subarachnoid hemorrhage: Preliminary results of a proteome-wide screening. J. Cereb. Blood Flow Metab. 27, 1675-1683.
13. Dayon, L., Hainard, A., Licker, V., Turck, N., Kuhn, K., Hochstrasser, D. F., Burkhard, P. R., and Sanchez, J. C. (2008) Relative quantification of proteins in human cerebrospinal fluids by MS/MS using 6-plex isobaric tags. Anal. Chem. 80, 2921-2931.
14. Thompson, A., Schafer, J., Kuhn, K., Kienle, S., Schwarz, J., Schmidt, G., Neumann, T., and Hamon, C. (2003) Tandem mass tags: A novel quantification strategy for comparative analysis of complex protein mixtures by MS/MS. Anal. Chem. 75, 1895-1904.
15. Lescuyer, P., Allard, L., Zimmermann-Ivol, C. G., Burgess, J. A., Hughes-Frutiger, S., Burkhard, P. R., Sanchez, J. C., and Hochstrasser, D. F. (2004) Identification of post-mortem cerebrospinal fluid proteins as potential biomarkers of ischemia and neurodegeneration. Proteomics 4, 2234-2241.
16. Tuck, M. K., Chan, D. W., Chia, D., Godwin, A. K., Grizzle, W. E., Krueger, K. E., Rom, W., Sanda, M., Sorbara, L., Stass, S., Wang, W., and Brenner, D. E. (2009) Standard operating procedures for serum and plasma collection: early detection research network consensus statement standard operating procedure integration working group. J Proteome Res 8, 113-117.
17. Bradford, M. M. (1976) Rapid and sensitive method for quantitation of microgram quantities of protein utilizing principle of protein-dye binding. Anal. Biochem. 72, 248-254.
18. Blum, H., Beier, H., and Gross, H. J. (1987) Improved silver staining of plant-proteins, RNA and DNA in polyacrylamide gels. Electrophoresis 8, 93-99.
19. Towbin, H., Staehelin, T., and Gordon, J. (1979) Electrophoretic transfer of proteins from polyacrylamide gels to nitrocellulose sheets—procedure and some applications. Proc. Natl. Acad. Sci. U.S.A. 76, 4350-4354.
20. Burgess, J. A., Lescuyer, P., Hainard, A., Burkhard, P. R., Turck, N., Michel, P., Rossier, J. S., Reymond, F., Hochstrasser, D. F., and Sanchez, J. C. (2006) Identification of brain cell death associated proteins in human post-mortem cerebrospinal fluid. J. Proteome Res. 5, 1674-1681.
21. Hainard, A., Tiberti, N., Robin, X., Lejon, V., Ngoyi, D. M., Matovu, E., Enyaru, J. C., Fouda, C., Ndung'u, J. M., Lisacek, F., Muller, M., Turck, N., and Sanchez, J. C. (2009) A Combined CXCL10, CXCL8 and H-FABP Panel for the Staging of Human African Trypanosomiasis Patients. PLoS Neglected Tropical Diseases 3, e459.
22. Horth, P., Miller, C. A., Preckel, T., and Wenz, C. (2006) Efficient fractionation and improved protein identification by peptide OFFGEL electrophoresis. Mol. Cell. Proteomics 5, 1968-1974.
23. Ros, A., Faupel, M., Mees, H., van Oostrum, J., Ferrigno, R., Reymond, F., Michel, P., Rossier, J. S., and Girault, H. H. (2002) Protein purification by Off-Gel electrophoresis. Proteomics 2, 151-156,
24. Dayon, L., Turck, N., Kienle, S., Schulz-Knappe, P., Hochstrasser, D. F., Scher!, A., and Sanchez, J. C. (2010) Isobaric tagging-based selection and quantitation of cerebrospinal fluid tryptic peptides with reporter calibration curves. Anal. Chem., DOI 10.1021/ac901854k.
25. Herrmann, M., Vos, P., Wunderlich, M. T., de Bruijn, C., and Lamers, K. J. B. (2000) Release of glial tissue-specific proteins after acute stroke—A comparative analysis of serum concentrations of protein S-100B and glial fibrillary acidic protein. Stroke 31, 2670-2677.
26. Jauch, E. C., Lindsell, C., Broderick, J., Fagan, S. C., Tilley, B. C., Levine, S. R., and Grp, N. r.-P. S. S. (2006) Association of serial biochemical markers with acute ischemic stroke—The National Institute of Neurological Disorders and Stroke recombinant tissue plasminogen activator Stroke Study. Stroke 37, 2508-2513.
27. Lamers, K. J. B., Vos, P., Verbeek, M. M., Rosmalen, F., van Geel, W. J. A., and van Engelen, B. G. M. (2003) Protein S-100B, neuron-specific enolase (NSE), myelin basic protein (MBP) and glial fibrillary acidic protein (GFAP) in cerebrospinal fluid (CSF) and blood of neurological patients. Brain Res. Bull. 61, 261-264.
28. Buttner, T., Weyers, S., Postert, T., Sprengelmeyer, R., and Kuhn, W. (1997) S-100 protein: Serum marker of focal brain damage after ischemic territorial MCA infarction. Stroke 28, 1961-1965.
29. Missler, U., Wiesmann, M., Friedrich, C., and Kaps, M. (1997) S-100 protein and neuron-specific enolase concentrations in blood as indicators of infarction volume and prognosis in acute ischemic stroke. Stroke 28, 1956-1960.
30. Berger, C., Dohmen, C., Maurer, M. H., Graf, R., and Schwab, S. (2004) Cerebral microdialysis in stroke. Nervenarzt 75, 113-123.
31. Zougman, A., Pilch, B., Podtelejnikov, A., Kiehntopf, M., Schnabel, C., Kurnar, C., and Mann, M. (2008) Integrated analysis of the cerebrospinal fluid peptidome and proteome. J. Proteome Res. 7, 386-399.
32. da Silva, S, F., Correa, C. L., Tortelote, G. G., Einicker-Lamas, M., Martinez, A. M. B., and Allodi, S. (2004) Glial fibrillary acidic protein (GFAP)-like immunoreactivity in the visual system of the crab Ucides cordatus (Crustacea, Decapoda). Biol. Cell 96, 727-734.
33. Kaufmann, A. M., Firlik, A. D., Fukui, M. B., Wechsler, L. R., Jungries, C. A., and Yonas, H. (1999) Ischemic core and penumbra in human stroke. Stroke 30, 93-99.
34. Rothermundt, M., Peters, M., Prehn, J. H. M., and Arolt, V. (2003) S100B in brain damage and neurodegeneration. Microsc. Res. Tech, 60, 614-632.
35. Wiesmann, M., Missler, U., Hagenstrom, H., and Gottmann, D. (1997) S-100 protein plasma levels after aneurysmal subarachnoid haemorrhage. Acta Neurochir. (Wien). 139, 1155-1160.
36. Romner, B., Ingebrigtsen, T., Kongstad, F., and Borgesen, S. E. (2000) Traumatic brain damage: Serum S-100 protein measurements related to neuroradiological findings. J. Neurotrauma 17, 641-647.
37. Sen, J., Belli, A., Petzold, A., Russo, S., Keir, G., Thompson, E. J., Smith, M., and Kitchen, N. (2005) Extracellular fluid S100B in the injured brain: a future surrogate marker of acute brain injury? Acta Neurochir. (Wien). 147, 897-900.
38. Salinas, A. E., and Wong, M. G. (1999) Glutathione S-transferases—A review. Curr. Med. Chem. 6, 279-309.
39. Theodore, C., Singh, S. V., Hong, T. D., and Awasthi, Y. C. (1985) Glutathione S-transferases of human brain. Evidence for two immunologically distinct types of 26500-Mr subunits. Biochem. J. 225, 375-382.
40. Shi, M., Bradner, J., Bammler, T. K., Eaton, D. L., Zhang, J. P., Ye, Z. C., Wilson, A. M., Montine, T. J., Pan, C., and Zhang, J. (2009) Identification of Glutathione S-Transferase Pi as a Protein Involved in Parkinson Disease Progression. Am. J. Pathol. 175, 54-65.
41. Rhee, S. G., Yang, K. S., Kang, S. W., Woo, H. A., and Chang, T. S. (2005) Controlled elimination of intracellular H2O2: Regulation of peroxiredoxin, catalase, and glutathione peroxidase via post-translational modification. Antioxidants & Redox Signaling 7, 619-626.
42. Wood, Z. A., Schroder, E., Harris, J. R., and Poole, L. B. (2003) Structure, mechanism and regulation of peroxiredoxins. Trends Biochem. Sci. 28, 32-40.
43. Power, J. H. T., Asad, S., Chataway, T. K., Chegini, F., Manavis, J., Temlett, J. A., Jensen, P. H., Blumbergs, P. C., and Gai, W. P. (2008) Peroxiredoxin 6 in human brain: molecular forms, cellular distribution and association with Alzheimer's disease pathology. Acta Neuropathol. (Berl). 115, 611-622.
44. Sarafian, T. A., Verity, M. A., Vinters, H. V., Shih, C. C. Y., Shi, L. R., Ji, X. D., Dong, L. P., and Shau, H. Y. (1999) Differential expression of peroxiredoxin subtypes in human brain cell types. J. Neurosci. Res. 56, 206-212.
45. Basso, M., Giraudo, S., Corpillo, D., Bergamasco, B., Lopiano, L., and Fasano, M. (2004) Proteome analysis of human substantia nigra in Parkinson's disease. Proteomics 4, 3943-3952.
46. Krapfenbauer, K., Engidawork, E., Cairns, N., Fountoulakis, M., and Lubec, G. (2003) Aberrant expression of peroxiredoxin subtypes in neurodegenerative disorders. Brain Res. 967, 152-160.
47. Schreibelt, G., van Horssen, J., Haseloff, R. F., Reijerkerk, A., van der Pol, S. M. A., Nieuwenhuizen, O., Krause, E., Blasig, I. E., Dijkstra, C. D., Ronken, E., and de Vries, H. E. (2008) Protective effects of peroxiredoxin-1 at the injured blood-brain barrier. Free Radic. Biol. Med. 45, 256-264.
48. Kim, Y. J., Lee, W. S., 1p, C., Chae, H. Z., Park, E. M., and Park, Y. M. (2006) Prx1 suppresses radiation-induced c-Jun NH2-terminal kinase signaling in lung cancer cells through interaction with the glutathione S-transferase Pi/c-Jun NH2-terminal kinase complex. Cancer Res. 66, 7136-7142.
49. Manevich, Y., Feinstein, S. I., and Fisher, A. B. (2004) Activation of the antioxidant enzyme 1-CYS peroxiredoxin requires glutathionylation mediated by heterodimerization with pi GST. Proc. Natl. Acad. Sci. U.S.A. 101, 3780-3785.
50. Ralat, L. A., Manevich, Y., Fisher, A. B., and Colman, R. F. (2006) Direct evidence for the formation of a complex between 1-cysteine peroxiredoxin and glutathione S-transferase pi with activity changes in both enzymes. Biochemistry (Mosc). 45, 360-372.

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1-42. (canceled)

43. A method of aiding the diagnosis of acute brain damage in a subject, the method comprising:
(i) assaying the concentration in a sample from the subject of at least one oxidative stress polypeptide selected from the group consisting of: PRDX1, PRDX6 and GSTP1; and
(ii) assaying the concentration in the sample from the subject of at least one further polypeptide selected from Panel A;
(iii) comparing the concentrations of (i) and (ii) to the concentrations of the polypeptides in a reference standard and determining quantitative ratios for the polypeptides; and
(iv) determining that a quantitative ratio of each of the assayed polypeptides in the sample to the polypeptides in the reference standard of greater than 1.3 indicates an increased likelihood of acute brain damage having occurred in the subject.

44. A method according to claim 43, wherein step (ii) comprises:
assaying the concentration of at least one further polypeptide selected from Panel B, Panel C, Panel 1, Panel 1H, Panel 1C, Panel 1A, Panel 1B, Panel 2, Panel 2A, or Panel 2B.

45. A method according to claim 43, wherein step (i) comprises:
(i) assaying the concentration of at least two oxidative stress polypeptides selected from the group consisting of: PRDX1, PRDX6 and GSTP1; or
(ii) assaying the concentration of each of the oxidative stress polypeptides PRDX1, PRDX6 and GSTP1.

46. A method according to claim 43, wherein step (ii) comprises assaying the concentration of:
(i) at least two further polypeptides selected from Panel A; or
(ii) at least four further polypeptides selected from Panel A.

47. A method according to claim 43, wherein the acute brain damage is stroke.

48. A method according claim 43, wherein the sample is brain microdialysate fluid, cerebrospinal fluid, or blood.

49. A method according claim 43, wherein step (i) comprises assaying the concentration of PRDX1 in a sample from the subject.

50. A method according to claim 43, wherein the polypeptide is assayed by:
western blotting, bead suspension array or by planar array, isobaric protein tagging or by isotopic protein tagging, or mass spectrometer-based assay.

51. An assay device, which comprises a solid substrate having a location containing a material, which recognizes, binds to or has affinity for a first and a second polypeptide or a fragment, variant, or mutant thereof, wherein the first polypeptide is selected from PRDX1, PRDX6 and GSTP1 and the second polypeptide is selected from Panel A.

52. An assay device, which comprises a solid substrate having a location containing a material, which recognizes, binds to or has affinity for a polypeptide, or a fragment, variant, or mutant thereof, wherein the polypeptide is selected from Panel 2.

53. An assay device according to claim 52, which has a unique addressable location for each antibody, thereby permitting an assay readout for each individual polypeptide or for any combination of polypeptides.

54. A method of diagnosis or prognostic monitoring of acute brain damage in a subject, said method comprising:
(a) obtaining and extracting proteins from a relevant tissue sample from an individual;
(b) digesting said proteins to produce a population of peptides;
(c) determining the abundance of one or more of said peptides listed in Table 14 using Selected Reaction Monitoring of one or more of the transitions listed in Table 15;
(d) comparing the abundance of said one or more peptides with a pre-determined peptide abundance associated with a diagnosis of acute brain damage; and
(e) determining whether the subject has suffered acute brain damage and/or that the acute brain damage is worsening or improving based on the differences in abundance of said one or more peptides.

55. A preparation comprising one or more synthetic peptides selected from the group listed in Table 14.

56. A preparation according to claim 55, wherein said one or more synthetic peptides are selected from
(i) the following peptides from GSTP1:
```
TFIVGDQISFADYNLLDLLLIHEVLAPGCLDAFPLLSAYVGR
          MPPYTVVYFPVR
          DDYVK
          DQQEAALVDMVNDGVEDLR
          FQDGDLTLYQSNTILR
          ASCLYGQLPK
          AFLASPEYVNLPINGNGK
          MLLADQGQSWK
          LSARPK
          TLGLYGK
          EEVVTVETWQEGSLK
          ALPGQLKPFETLLSQNQGGK
          YISLIYTNYEAGK;
```
(ii) the following peptides from PRDX1:
HGEVCPAGWKPGSDTIKPDVQK
QGGLGPMNIPLVSDPK
ADEGISFR
DISLSDYK
LVQAFQFTDK
IGHPAPNFK
LNCQVIGASVDSHFCHLAWVNTPK
YVVFFFYPLDFTFVCPTEIIAFSDR
MSSGNAK
TIAQDYGVLK
ATAVMPDGQFK;
(iii) from the following peptides from PRDX6:
GMPVTAR
MPGGLLLGDVAPNFEANTTVGR
DFTPVCTTELGR
VVFVFGPDK
LIALSIDSVEDHLAWSK
ELAILLGMLDPAEK
LSILYPATTGR
VATPVDWK
NFDEILR
LPFPIIDDR
VVISLQLTAEK
DINAYNCEEPTEK
LAPEFAK
DGDSVMVLPTIPEEEAK
FHDFLGDSWGILFSHPR;
(iv) from the following peptides from DDAH1:
ALPESLGQHALR
DENATLDGGDVLFTGR
DYAVSTVPVADGLHLK
GAEILADTFK
GEEVDVAR
QHQLYVGVLGSK
TPEEYPESAK;
(v) from the following peptides from CYTB:
HDELTYF
SQVVAGTNYFIK
VFQSLPHENKPLTLSNYQTNK
VHVGDEDFVHLR;
(vi) from the following peptides from ACBP:
MSQAEFEK
AAEEVR
QATVGDINTERPGMLDFTGK
TKPSDEEMLFIYGHYK
WDAWNELK
MWGDLWLLPPASANPGTGTEAEFEK
MPAFAEFEK;
(vii) from the following peptides from CSRP1:
GFGFGQGAGALVHSE
GLESTTLADK
GYGYGQGAGTLSTDK;
(viii) from the following peptides from MT3:
GGEAAEAEAEK
MDPETCPCPSGGSCTCADSCK
SCCSCCPAECEK;
and
(ix) from the following peptides from PEPB1:
GNDISSGTVLSDYVGSGPPK
LYEQLSGK
LYTLVLTDPDAPSR
NRPTSISWDGLDSGK
VLTPTQVK
YVWLVYEQDRPLK.

57. A preparation according to claim 55, wherein each peptide contains one or more stable heavy isotopes selected from hydrogen, carbon, oxygen, nitrogen or sulphur.
